SEQUENCE LISTING (76 sequences)

SEQ ID NO: 1 - protein, 405 aa, Zika virus
IRCIGVSNRDFVEGMSGGTWVDIVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGS

SEQ ID NO: 2 - protein, 405 aa, synthetic construct
IRCICVSNRDFVEGMSGGTWVDIVLEHGGCVTVMAQDKPTVDIELVTTTVSNMAEVRSYCYEASISDMASDSRCPTQGEAYLDKQSDTQYVCKRTLVDRGWCNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGS

SEQ ID NO: 3 - DNA, 1215 bp, Zika virus
  source 1..1215: mol_type = unassigned DNA
atcaggtgcatcggcgtgtccaatagagacttcgtggagggcatgtctggcggcacctgggtggatatcgtgctggagcacggcggctgcgtgacagtgatggcccaggacaagcccaccgtggatatcgagctggtgaccacaaccgtgtccaacatggccgaggtgaggtcttactgctatgaggcctctatcagcgacatggcctccgattctcgctgtcctacccagggcgaggcctacctggacaagcagtccgatacacagtacgtgtgcaagaggaccctggtggacaggggatggggaaatggatgtggcctgttcggcaagggctctctggtgacatgcgccaagtttgcctgtagcaagaagatgaccggcaagtccatccagcctgagaacctggagtatcggatcatgctgagcgtgcacggcagccagcactccggcatgatcgtgaacgacacaggccacgagacagatgagaatcgggccaaggtggagatcacacctaacagcccaagagcagaggccaccctgggaggattcggctccctgggcctggactgcgagccaaggacaggcctggacttttccgatctgtactatctgaccatgaacaataagcactggctggtgcacaaggagtggttccacgacatcccactgccctggcacgcaggagcagatacaggaaccccacactggaacaataaggaggccctggtggagtttaaggatgcccacgccaagcggcagacagtggtggtgctgggcagccaggagggagcagtgcacaccgccctggcaggcgccctggaggcagagatggacggagcaaagggcagactgagctccggccacctgaagtgcaggctgaagatggataagctgcgcctgaagggcgtgagctactccctgtgcacagccgccttcaccttcaccaagatccccgccgagacactgcacggcacagtgaccgtggaggtgcagtatgccggcacagacggcccctgtaaggtgcctgcccagatggccgtggatatgcagacactgacccctgtgggcaggctgatcaccgccaatccagtgatcacagagtctaccgagaacagcaagatgatgctggagctggaccccccttttggcgattcctacatcgtgatcggcgtgggcgagaagaagatcacacaccactggcaccgctctggcagc

SEQ ID NO: 4 - DNA, 1215 bp, synthetic construct
  source 1..1215: mol_type = other DNA
atcaggtgcatctgcgtgtccaatagagacttcgtggagggcatgtctggcggcacctgggtggatatcgtgctggagcacggcggctgcgtgacagtgatggcccaggacaagcccaccgtggatatcgagctggtgaccacaaccgtgtccaacatggccgaggtgaggtcttactgctatgaggcctctatcagcgacatggcctccgattctcgctgtcctacccagggcgaggcctacctggacaagcagtccgatacacagtacgtgtgcaagaggaccctggtggacaggggatggtgcaatggatgtggcctgttcggcaagggctctctggtgacatgcgccaagtttgcctgtagcaagaagatgaccggcaagtccatccagcctgagaacctggagtatcggatcatgctgagcgtgcacggcagccagcactccggcatgatcgtgaacgacacaggccacgagacagatgagaatcgggccaaggtggagatcacacctaacagcccaagagcagaggccaccctgggaggattcggctccctgggcctggactgcgagccaaggacaggcctggacttttccgatctgtactatctgaccatgaacaataagcactggctggtgcacaaggagtggttccacgacatcccactgccctggcacgcaggagcagatacaggaaccccacactggaacaataaggaggccctggtggagtttaaggatgcccacgccaagcggcagacagtggtggtgctgggcagccaggagggagcagtgcacaccgccctggcaggcgccctggaggcagagatggacggagcaaagggcagactgagctccggccacctgaagtgcaggctgaagatggataagctgcgcctgaagggcgtgagctactccctgtgcacagccgccttcaccttcaccaagatccccgccgagacactgcacggcacagtgaccgtggaggtgcagtatgccggcacagacggcccctgtaaggtgcctgcccagatggccgtggatatgcagacactgacccctgtgggcaggctgatcaccgccaatccagtgatcacagagtctaccgagaacagcaagatgatgctggagctggaccccccttttggcgattcctacatcgtgatcggcgtgggcgagaagaagatcacacaccactggcaccgctctggcagc

SEQ ID NO: 5 - RNA, 1215 nt, Zika virus
  source 1..1215: mol_type = unassigned RNA
atcaggtgcatcggcgtgtccaatagagacttcgtggagggcatgtctggcggcacctgggtggatatcgtgctggagcacggcggctgcgtgacagtgatggcccaggacaagcccaccgtggatatcgagctggtgaccacaaccgtgtccaacatggccgaggtgaggtcttactgctatgaggcctctatcagcgacatggcctccgattctcgctgtcctacccagggcgaggcctacctggacaagcagtccgatacacagtacgtgtgcaagaggaccctggtggacaggggatggggaaatggatgtggcctgttcggcaagggctctctggtgacatgcgccaagtttgcctgtagcaagaagatgaccggcaagtccatccagcctgagaacctggagtatcggatcatgctgagcgtgcacggcagccagcactccggcatgatcgtgaacgacacaggccacgagacagatgagaatcgggccaaggtggagatcacacctaacagcccaagagcagaggccaccctgggaggattcggctccctgggcctggactgcgagccaaggacaggcctggacttttccgatctgtactatctgaccatgaacaataagcactggctggtgcacaaggagtggttccacgacatcccactgccctggcacgcaggagcagatacaggaaccccacactggaacaataaggaggccctggtggagtttaaggatgcccacgccaagcggcagacagtggtggtgctgggcagccaggagggagcagtgcacaccgccctggcaggcgccctggaggcagagatggacggagcaaagggcagactgagctccggccacctgaagtgcaggctgaagatggataagctgcgcctgaagggcgtgagctactccctgtgcacagccgccttcaccttcaccaagatccccgccgagacactgcacggcacagtgaccgtggaggtgcagtatgccggcacagacggcccctgtaaggtgcctgcccagatggccgtggatatgcagacactgacccctgtgggcaggctgatcaccgccaatccagtgatcacagagtctaccgagaacagcaagatgatgctggagctggaccccccttttggcgattcctacatcgtgatcggcgtgggcgagaagaagatcacacaccactggcaccgctctggcagc

SEQ ID NO: 6 - RNA, 1215 nt, synthetic construct
  source 1..1215: mol_type = other RNA
atcaggtgcatctgcgtgtccaatagagacttcgtggagggcatgtctggcggcacctgggtggatatcgtgctggagcacggcggctgcgtgacagtgatggcccaggacaagcccaccgtggatatcgagctggtgaccacaaccgtgtccaacatggccgaggtgaggtcttactgctatgaggcctctatcagcgacatggcctccgattctcgctgtcctacccagggcgaggcctacctggacaagcagtccgatacacagtacgtgtgcaagaggaccctggtggacaggggatggtgcaatggatgtggcctgttcggcaagggctctctggtgacatgcgccaagtttgcctgtagcaagaagatgaccggcaagtccatccagcctgagaacctggagtatcggatcatgctgagcgtgcacggcagccagcactccggcatgatcgtgaacgacacaggccacgagacagatgagaatcgggccaaggtggagatcacacctaacagcccaagagcagaggccaccctgggaggattcggctccctgggcctggactgcgagccaaggacaggcctggacttttccgatctgtactatctgaccatgaacaataagcactggctggtgcacaaggagtggttccacgacatcccactgccctggcacgcaggagcagatacaggaaccccacactggaacaataaggaggccctggtggagtttaaggatgcccacgccaagcggcagacagtggtggtgctgggcagccaggagggagcagtgcacaccgccctggcaggcgccctggaggcagagatggacggagcaaagggcagactgagctccggccacctgaagtgcaggctgaagatggataagctgcgcctgaagggcgtgagctactccctgtgcacagccgccttcaccttcaccaagatccccgccgagacactgcacggcacagtgaccgtggaggtgcagtatgccggcacagacggcccctgtaaggtgcctgcccagatggccgtggatatgcagacactgacccctgtgggcaggctgatcaccgccaatccagtgatcacagagtctaccgagaacagcaagatgatgctggagctggaccccccttttggcgattcctacatcgtgatcggcgtgggcgagaagaagatcacacaccactggcaccgctctggcagc

SEQ ID NO: 7 - protein, 402 aa, West Nile virus
FNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGS

SEQ ID NO: 8 - protein, 402 aa, synthetic construct
FNCLCMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWCNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGS

SEQ ID NO: 9 - DNA, 1200 bp, Synthetic construct
  source 1..1200: mol_type = other DNA
tttaactgcctgtgcatgagcaaccgcgattttctggaaggcgtgagcggcgcgacctgggtggatctggtgctggaaggcgatagctgcgtgaccattatgagcaaagataaaccgaccattgatgtgaaaatgatgaacatggaagcggcgaacctggcggaagtgcgcagctattgctatctggcgaccgtgagcgatctgagcaccaaagcggcgtgcccgaccatgggcgaagcgcataacgataaacgcgcggatccggcgtttgtgtgccgccagggcgtggtggatcgcggctggtgcaacggctgcggcctgtttggcaaaggcagcattgatacctgcgcgaaatttgcgtgcagcaccaaagcgattggccgcaccattctgaaagaaaacattaaatatgaagtggcgatttttgtgcatggcccgaccaccgtggaaagccatggcaactatagcacccaggtgggcgcgacccaggcgggccgctttagcattaccccggcggcgccgagctataccctgaaactgggcgaatatggcgaagtgaccgtggattgcgaaccgcgcagcggcattgataccaacgcgtattatgtgatgaccgtgggcaccaaaacctttctggtgcatcgcgaatggtttatggatctgaacctgccgtggagcagcgcgggcagcaccgtgtggcgcaaccgcgaaaccctgatggaatttgaagaaccgcatgcgaccaaacagagcgtgattgcgctgggcagccaggaaggcgcgctgcatcaggcgctggcgggcgcgattccggtggaatttagcagcaacaccgtgaaactgaccagcggccatctgaaatgccgcgtgaaaatggaaaaactgcagctgaaaggcaccacctatggcgtgtgcagcaaagcgtttaaatttctgggcaccccggcggataccggccatggcaccgtggtgctggaactgcagtataccggcaccgatggcccgtgcaaagtgccgattagcagcgtggcgagcctgaacgatctgaccccggtgggccgcctggtgaccgtgaacccgtttgtgagcgtggcgaccgcgaacgcgaaagtgctgattgaactggaaccgccgtttggcgatagctatattgtggtgggccgcggcgaacagcagattaaccatcattggcataaaagc

SEQ ID NO: 10 - RNA, 1206 nt, Synthetic construct
  source 1..1206: mol_type = other RNA
tttaactgcctgtgcatgagcaaccgcgattttctggaaggcgtgagcggcgcgacctgggtggatctggtgctggaaggcgatagctgcgtgaccattatgagcaaagataaaccgaccattgatgtgaaaatgatgaacatggaagcggcgaacctggcggaagtgcgcagctattgctatctggcgaccgtgagcgatctgagcaccaaagcggcgtgcccgaccatgggcgaagcgcataacgataaacgcgcggatccggcgtttgtgtgccgccagggcgtggtggatcgcggctggtgcaacggctgcggcctgtttggcaaaggcagcattgatacctgcgcgaaatttgcgtgcagcaccaaagcgattggccgcaccattctgaaagaaaacattaaatatgaagtggcgatttttgtgcatggcccgaccaccgtggaaagccatggcaactatagcacccaggtgggcgcgacccaggcgggccgctttagcattaccccggcggcgccgagctataccctgaaactgggcgaatatggcgaagtgaccgtggattgcgaaccgcgcagcggcattgataccaacgcgtattatgtgatgaccgtgggcaccaaaacctttctggtgcatcgcgaatggtttatggatctgaacctgccgtggagcagcgcgggcagcaccgtgtggcgcaaccgcgaaaccctgatggaatttgaagaaccgcatgcgaccaaacagagcgtgattgcgctgggcagccaggaaggcgcgctgcatcaggcgctggcgggcgcgattccggtggaatttagcagcaacaccgtgaaactgaccagcggccatctgaaatgccgcgtgaaaatggaaaaactgcagctgaaaggcaccacctatggcgtgtgcagcaaagcgtttaaatttctgggcaccccggcggataccggccatggcaccgtggtgctggaactgcagtataccggcaccgatggcccgtgcaaagtgccgattagcagcgtggcgagcctgaacgatctgaccccggtgggccgcctggtgaccgtgaacccgtttgtgagcgtggcgaccgcgaacgcgaaagtgctgattgaactggaaccgccgtttggcgatagctatattgtggtgggccgcggcgaacagcagattaaccatcattggcataaaagcggcagc

SEQ ID NO: 11 - protein, 10 aa, Synthetic construct
EQKLISEEDL

SEQ ID NO: 12 - protein, 6 aa, Synthetic construct
HHHHHH

SEQ ID NO: 13 - protein, 18 aa, synthetic construct
MKLCILLAVVAFVGLSLG

SEQ ID NO: 14 - DNA, 54 bp, Synthetic construct
  source 1..54: mol_type = other DNA
atgaagttatgcatattactggccgtcgtggcctttgttggcctctcgctcggg

SEQ ID NO: 15 - protein, 429 aa, Synthetic construct
GSGEQKLISEEDLGGGGSFNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSGTGHHHHHH

SEQ ID NO: 16 - DNA, 1296 bp, synthetic construct
  source 1..1296: mol_type = other DNA
agatctggatcgggcgagcagaaactcatctctgaagaagatttgggcggcggcggttcgttcaattgtctgggaatgtccaaccgggactttctggaaggtgtttccggtgccacgtgggtggatttggtcttggaaggcgactcatgtgtaaccataatgagcaaagataaacctaccattgacgtgaaaatgatgaatatggaagctgcaaacttggccgaggtgagatcgtattgttatctggccactgtgagtgatctttccacaaaagcggcctgtcctaccatgggtgaagcccacaacgataagcgtgccgacccggcctttgtttgtcggcaaggagtggtcgaccggggatggggcaatggatgtggtctgttcggcaagggttcaatcgacacatgcgcaaaattcgcctgctccacgaaggcaatcggaagaacaattctaaaggaaaatataaaatacgaggttgccatatttgtgcacggcccgactacagtcgaaagccacggaaactattcgactcaagtaggagcgacgcaagctggtagatttagcataactccggccgccccctcatataccttgaagctcggtgaatacggagaagtcaccgtagattgcgaaccgcgatcaggaatcgatacgaatgcatactatgttatgacagttggcactaaaacattcctggtccatcgcgagtggtttatggacctaaatctcccctggtctagtgcgggttcgacagtttggcgaaatagagagacgttgatggaattcgaagaaccccatgccactaagcagtcggtaattgccctcggttcgcaggaaggagctctgcaccaggcactagccggagcgataccagtggaattttccagcaataccgtcaaacttacgagcggccacctgaagtgtcgggtaaagatggaaaagttgcaattgaaaggaacaacgtatggtgtctgttctaaagctttcaaatttctgggtacaccagcggataccggccacggcaccgtcgtgcttgaactccaatatactggcaccgacggaccctgtaaagttccgatctcatcagtagcgagcctgaacgacttgacaccagtgggacgactcgtgactgttaatccctttgtcagcgttgctaccgcgaatgccaaggtcctgatagaactggaaccaccctttggagactcttatatcgtggtcggaaggggagagcaacaaatcaatcaccactggcataagtcgggctcgggtaccggtcatcatcaccatcaccattga

SEQ ID NO: 17 - protein, 429 aa, synthetic construct
GSGEQKLISEEDLGGGGSFNCLCMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWCNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSGTGHHHHHH

SEQ ID NO: 18 - DNA, 1296 bp, synthetic construct
  source 1..1296: mol_type = other DNA
agatctggaagcggcgagcagaaattgatttcagaagaagacctgggcggtggcggatcattcaactgtttgtgtatgtccaacagggattttctggaaggtgtctccggagcaacgtgggtcgacttggtactagagggagattcgtgcgtaactataatgtccaaagataagcccaccattgatgtgaaaatgatgaatatggaagcggctaatctagcagaggtgcgttcatattgctatcttgccacagttagtgatttgtccaccaaagccgcgtgtcccacgatgggagaagcccataacgataaaagagcggatccggcttttgtctgtaggcaaggtgtcgttgaccgaggttggtgtaacggatgcggcctgttcggaaaaggatctatcgacacatgtgccaaattcgcctgttccaccaaggctattggccggacaatcctcaaagaaaatatcaaatatgaagttgctatattcgtccacggcccgacaactgttgagagtcatggcaactatagcacgcaagtgggtgccactcaagccggacgcttttccatcaccccagctgcgccctcatacacccttaagctgggtgaatacggagaagtcacagttgactgcgaacctaggtcaggcatcgataccaacgcatattatgtaatgaccgtgggtacaaagacgtttctcgtgcaccgtgaatggtttatggacctcaacctgccatggagcagcgccggtagcaccgtctggcgcaatcgcgaaactctaatggaattcgaagaaccacatgcgaccaagcagtccgttattgccctgggcagccaagagggcgcgctgcatcaagccttggcgggtgcgatacccgttgagtttagttccaacacggtcaaacttacgtccggacatttgaaatgtcgggtgaaaatggagaaacttcagctcaaaggcactacctatggagtctgtagcaaggcctttaaatttctgggtaccccggctgatactggtcacggaacagtggttcttgaacttcaatatacaggaacggacggcccatgtaaagttccaattagctccgtagctagcctgaacgacttgacccctgtgggtcgattggtgactgtcaatcccttcgtgtccgtagctacagcaaatgctaaagtattgattgaactggagccgccgttcggcgactcctacatcgtcgtgggaaggggtgaacaacaaatcaaccatcattggcacaagtccggatcgggtaccggtcatcatcaccatcaccattga

SEQ ID NO: 19 - RNA, 1296 nt, Synthetic construct
  source 1..1296: mol_type = other RNA
agatctggaagcggcgagcagaaattgatttcagaagaagacctgggcggtggcggatcattcaactgtttgtgtatgtccaacagggattttctggaaggtgtctccggagcaacgtgggtcgacttggtactagagggagattcgtgcgtaactataatgtccaaagataagcccaccattgatgtgaaaatgatgaatatggaagcggctaatctagcagaggtgcgttcatattgctatcttgccacagttagtgatttgtccaccaaagccgcgtgtcccacgatgggagaagcccataacgataaaagagcggatccggcttttgtctgtaggcaaggtgtcgttgaccgaggttggtgtaacggatgcggcctgttcggaaaaggatctatcgacacatgtgccaaattcgcctgttccaccaaggctattggccggacaatcctcaaagaaaatatcaaatatgaagttgctatattcgtccacggcccgacaactgttgagagtcatggcaactatagcacgcaagtgggtgccactcaagccggacgcttttccatcaccccagctgcgccctcatacacccttaagctgggtgaatacggagaagtcacagttgactgcgaacctaggtcaggcatcgataccaacgcatattatgtaatgaccgtgggtacaaagacgtttctcgtgcaccgtgaatggtttatggacctcaacctgccatggagcagcgccggtagcaccgtctggcgcaatcgcgaaactctaatggaattcgaagaaccacatgcgaccaagcagtccgttattgccctgggcagccaagagggcgcgctgcatcaagccttggcgggtgcgatacccgttgagtttagttccaacacggtcaaacttacgtccggacatttgaaatgtcgggtgaaaatggagaaacttcagctcaaaggcactacctatggagtctgtagcaaggcctttaaatttctgggtaccccggctgatactggtcacggaacagtggttcttgaacttcaatatacaggaacggacggcccatgtaaagttccaattagctccgtagctagcctgaacgacttgacccctgtgggtcgattggtgactgtcaatcccttcgtgtccgtagctacagcaaatgctaaagtattgattgaactggagccgccgttcggcgactcctacatcgtcgtgggaaggggtgaacaacaaatcaaccatcattggcacaagtccggatcgggtaccggtcatcatcaccatcaccattga

SEQ ID NO: 20 - protein, 394 aa, Dengue virus
MRCVGVGNRDFVEGLSGATWVDVVLEHGGCVTTMAKNKPTLDIELQKTEATQLATLRKLCIEGKITNVTTDSRCPTQGEAILPEEQDQNYVCKHTYVDRGWGNGCGLFGKGSLVTCAKFQCLESIEGKAVQHENLKYTVIITVHTGDQHQVGNETQGVTAEITPQASTVEAILPEYGTLGLECSPRTGLDFNEMILLTMKNKAWMVHRQWFFDLPLPWTSGATTETPTWNKKELLVTFKNAHAKKQEVVVLGSQEGAMHTALTGATEIQTSGGTSIFAGHLKCRLKMDKLELKGMSYAMCLNAFVLKKEVSETQHGTILIKVEYKGEDAPCKIPFSTEDGQGKAHNGRLITANPIVTKKEEPVNIEAEPPFGESNIVIGIGDKALKINWYKKGS

SEQ ID NO: 21 - protein, 394 aa, Synthetic construct
MRCVCVGNRDFVEGLSGATWVDVVLEHGGCVTTMAKNKPTLDIELQKTEATQLATLRKLCIEGKITNVTTDSRCPTQGEAILPEEQDQNYVCKHTYVDRGWCNGCGLFGKGSLVTCAKFQCLESIEGKAVQHENLKYTVIITVHTGDQHQVGNETQGVTAEITPQASTVEAILPEYGTLGLECSPRTGLDFNEMILLTMKNKAWMVHRQWFFDLPLPWTSGATTETPTWNKKELLVTFKNAHAKKQEVVVLGSQEGAMHTALTGATEIQTSGGTSIFAGHLKCRLKMDKLELKGMSYAMCLNAFVLKKEVSETQHGTILIKVEYKGEDAPCKIPFSTEDGQGKAHNGRLITANPIVTKKEEPVNIEAEPPFGESNIVIGIGDKALKINWYKKGS

SEQ ID NO: 22 - DNA, 1182 bp, Synthetic construct
  source 1..1182: mol_type = other DNA
atgcgctgcgtgtgcgtgggcaaccgcgattttgtggaaggcctgagcggcgcgacctgggtggatgtggtgctggaacatggcggctgcgtgaccaccatggcgaaaaacaaaccgaccctggatattgaactgcagaaaaccgaagcgacccagctggcgaccctgcgcaaactgtgcattgaaggcaaaattaccaacgtgaccaccgatagccgctgcccgacccagggcgaagcgattctgccggaagaacaggatcagaactatgtgtgcaaacatacctatgtggatcgcggctggtgcaacggctgcggcctgtttggcaaaggcagcctggtgacctgcgcgaaatttcagtgcctggaaagcattgaaggcaaagcggtgcagcatgaaaacctgaaatataccgtgattattaccgtgcataccggcgatcagcatcaggtgggcaacgaaacccagggcgtgaccgcggaaattaccccgcaggcgagcaccgtggaagcgattctgccggaatatggcaccctgggcctggaatgcagcccgcgcaccggcctggattttaacgaaatgattctgctgaccatgaaaaacaaagcgtggatggtgcatcgccagtggttttttgatctgccgctgccgtggaccagcggcgcgaccaccgaaaccccgacctggaacaaaaaagaactgctggtgacctttaaaaacgcgcatgcgaaaaaacaggaagtggtggtgctgggcagccaggaaggcgcgatgcataccgcgctgaccggcgcgaccgaaattcagaccagcggcggcaccagcatttttgcgggccatctgaaatgccgcctgaaaatggataaactggaactgaaaggcatgagctatgcgatgtgcctgaacgcgtttgtgctgaaaaaagaagtgagcgaaacccagcatggcaccattctgattaaagtggaatataaaggcgaagatgcgccgtgcaaaattccgtttagcaccgaagatggccagggcaaagcgcataacggccgcctgattaccgcgaacccgattgtgaccaaaaaagaagaaccggtgaacattgaagcggaaccgccgtttggcgaaagcaacattgtgattggcattggcgataaagcgctgaaaattaactggtataaaaaaggcagc

SEQ ID NO: 23 - RNA, 1182 nt, Synthetic construct
  source 1..1182: mol_type = other RNA
atgcgctgcgtgtgcgtgggcaaccgcgattttgtggaaggcctgagcggcgcgacctgggtggatgtggtgctggaacatggcggctgcgtgaccaccatggcgaaaaacaaaccgaccctggatattgaactgcagaaaaccgaagcgacccagctggcgaccctgcgcaaactgtgcattgaaggcaaaattaccaacgtgaccaccgatagccgctgcccgacccagggcgaagcgattctgccggaagaacaggatcagaactatgtgtgcaaacatacctatgtggatcgcggctggtgcaacggctgcggcctgtttggcaaaggcagcctggtgacctgcgcgaaatttcagtgcctggaaagcattgaaggcaaagcggtgcagcatgaaaacctgaaatataccgtgattattaccgtgcataccggcgatcagcatcaggtgggcaacgaaacccagggcgtgaccgcggaaattaccccgcaggcgagcaccgtggaagcgattctgccggaatatggcaccctgggcctggaatgcagcccgcgcaccggcctggattttaacgaaatgattctgctgaccatgaaaaacaaagcgtggatggtgcatcgccagtggttttttgatctgccgctgccgtggaccagcggcgcgaccaccgaaaccccgacctggaacaaaaaagaactgctggtgacctttaaaaacgcgcatgcgaaaaaacaggaagtggtggtgctgggcagccaggaaggcgcgatgcataccgcgctgaccggcgcgaccgaaattcagaccagcggcggcaccagcatttttgcgggccatctgaaatgccgcctgaaaatggataaactggaactgaaaggcatgagctatgcgatgtgcctgaacgcgtttgtgctgaaaaaagaagtgagcgaaacccagcatggcaccattctgattaaagtggaatataaaggcgaagatgcgccgtgcaaaattccgtttagcaccgaagatggccagggcaaagcgcataacggccgcctgattaccgcgaacccgattgtgaccaaaaaagaagaaccggtgaacattgaagcggaaccgccgtttggcgaaagcaacattgtgattggcattggcgataaagcgctgaaaattaactggtataaaaaaggcagc

SEQ ID NO: 24 - protein, 396 aa, Dengue virus
MRCIGISNRDFVEGVSGGSWVDIVLEHGSCVTTMAKNKPTLDFELIKTEAKHPATLRKYCIEAKLTNTTTASRCPTQGEPSLNEEQDKRFICKHSMVDRGWGNGCGLFGKGGIVTCAMFTCKKNMEGKVVQPENLEYTIVITPHSGEEKAVGNDTGKHGTEIKVTPQSSITEAELTGYGTVTMECSPRTGLDFNEMVLLQMEDKAWLVHRQWFLDLPLPWLPGADKQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIVIGVEPGQLKLSWFKKGS

SEQ ID NO: 25 - protein, 396 aa, Synthetic construct
MRCICISNRDFVEGVSGGSWVDIVLEHGSCVTTMAKNKPTLDFELIKTEAKHPATLRKYCIEAKLTNTTTASRCPTQGEPSLNEEQDKRFICKHSMVDRGWCNGCGLFGKGGIVTCAMFTCKKNMEGKVVQPENLEYTIVITPHSGEEKAVGNDTGKHGTEIKVTPQSSITEAELTGYGTVTMECSPRTGLDFNEMVLLQMEDKAWLVHRQWFLDLPLPWLPGADKQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIVIGVEPGQLKLSWFKKGS

SEQ ID NO: 26 - DNA, 1188 bp, Synthetic construct
  source 1..1188: mol_type = other DNA
atgcgctgcatttgcattagcaaccgcgattttgtggaaggcgtgagcggcggcagctgggtggatattgtgctggaacatggcagctgcgtgaccaccatggcgaaaaacaaaccgaccctggattttgaactgattaaaaccgaagcgaaacatccggcgaccctgcgcaaatattgcattgaagcgaaactgaccaacaccaccaccgcgagccgctgcccgacccagggcgaaccgagcctgaacgaagaacaggataaacgctttatttgcaaacatagcatggtggatcgcggctggtgcaacggctgcggcctgtttggcaaaggcggcattgtgacctgcgcgatgtttacctgcaaaaaaaacatggaaggcaaagtggtgcagccggaaaacctggaatataccattgtgattaccccgcatagcggcgaagaaaaagcggtgggcaacgataccggcaaacatggcaccgaaattaaagtgaccccgcagagcagcattaccgaagcggaactgaccggctatggcaccgtgaccatggaatgcagcccgcgcaccggcctggattttaacgaaatggtgctgctgcagatggaagataaagcgtggctggtgcatcgccagtggtttctggatctgccgctgccgtggctgccgggcgcggataaacagggcagcaactggattcagaaagaaaccctggtgacctttaaaaacccgcatgcgaaaaaacaggatgtggtggtgctgggcagccaggaaggcgcgatgcataccgcgctgaccggcgcgaccgaaattcagatgagcagcggcaacctgctgtttaccggccatctgaaatgccgcctgcgcatggataaactgcagctgaaaggcatgagctatagcatgtgcaccggcaaatttaaagtggtgaaagaaattgcggaaacccagcatggcaccattgtgattcgcgtgcagtatgaaggcgatggcagcccgtgcaaaattccgtttgaaattatggatctggaaaaacgccatgtgctgggccgcctgattaccgtgaacccgattgtgaccgaaaaagatagcccggtgaacattgaagcggaaccgccgtttggcgatagctatattgtgattggcgtggaaccgggccagctgaaactgagctggtttaaaaaaggcagc

SEQ ID NO: 27 - RNA, 1188 nt, Synthetic construct
  source 1..1188: mol_type = other RNA
atgcgctgcatttgcattagcaaccgcgattttgtggaaggcgtgagcggcggcagctgggtggatattgtgctggaacatggcagctgcgtgaccaccatggcgaaaaacaaaccgaccctggattttgaactgattaaaaccgaagcgaaacatccggcgaccctgcgcaaatattgcattgaagcgaaactgaccaacaccaccaccgcgagccgctgcccgacccagggcgaaccgagcctgaacgaagaacaggataaacgctttatttgcaaacatagcatggtggatcgcggctggtgcaacggctgcggcctgtttggcaaaggcggcattgtgacctgcgcgatgtttacctgcaaaaaaaacatggaaggcaaagtggtgcagccggaaaacctggaatataccattgtgattaccccgcatagcggcgaagaaaaagcggtgggcaacgataccggcaaacatggcaccgaaattaaagtgaccccgcagagcagcattaccgaagcggaactgaccggctatggcaccgtgaccatggaatgcagcccgcgcaccggcctggattttaacgaaatggtgctgctgcagatggaagataaagcgtggctggtgcatcgccagtggtttctggatctgccgctgccgtggctgccgggcgcggataaacagggcagcaactggattcagaaagaaaccctggtgacctttaaaaacccgcatgcgaaaaaacaggatgtggtggtgctgggcagccaggaaggcgcgatgcataccgcgctgaccggcgcgaccgaaattcagatgagcagcggcaacctgctgtttaccggccatctgaaatgccgcctgcgcatggataaactgcagctgaaaggcatgagctatagcatgtgcaccggcaaatttaaagtggtgaaagaaattgcggaaacccagcatggcaccattgtgattcgcgtgcagtatgaaggcgatggcagcccgtgcaaaattccgtttgaaattatggatctggaaaaacgccatgtgctgggccgcctgattaccgtgaacccgattgtgaccgaaaaagatagcccggtgaacattgaagcggaaccgccgtttggcgatagctatattgtgattggcgtggaaccgggccagctgaaactgagctggtttaaaaaaggcagc

SEQ ID NO: 28 - protein, 396 aa, Dengue virus
MRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGVSYVMCTGSFKLEKEVAETQHGTVLVQVKYEGTDAPCKIPFSSQDEKGVTQNGRLITANPIVTDKEKPVNIEAEPPFGESYLVVGAGEKALKLSWFKKGS

SEQ ID NO: 29 - protein, 396 aa, synthetic construct
MRCVCIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWCNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGVSYVMCTGSFKLEKEVAETQHGTVLVQVKYEGTDAPCKIPFSSQDEKGVTQNGRLITANPIVTDKEKPVNIEAEPPFGESYLVVGAGEKALKLSWFKKGS

SEQ ID NO: 30 - DNA, 1188 bp, synthetic construct
  source 1..1188: mol_type = other DNA
atgcgctgcgtgtgcattggcaaccgcgattttgtggaaggcctgagcggcgcgacctgggtggatgtggtgctggaacatggcagctgcgtgaccaccatggcgaaagataaaccgaccctggatattgaactgctgaaaaccgaagtgaccaacccggcggtgctgcgcaaactgtgcattgaagcgaaaattagcaacaccaccaccgatagccgctgcccgacccagggcgaagcgaccctggtggaagaacaggataccaactttgtgtgccgccgcacctttgtggatcgcggctggtgcaacggctgcggcctgtttggcaaaggcagcctgattacctgcgcgaaatttaaatgcgtgaccaaactggaaggcaaaattgtgcagtatgaaaacctgaaatatagcgtgattgtgaccgtgcataccggcgatcagcatcaggtgggcaacgaaaccaccgaacatggcaccaccgcgaccattaccccgcaggcgccgaccagcgaaattcagctgaccgattatggcgcgctgaccctggattgcagcccgcgcaccggcctggattttaacgaaatggtgctgctgaccatgaaagaaaaaagctggctggtgcataaacagtggtttctggatctgccgctgccgtggaccagcggcgcgagcaccagccaggaaacctggaaccgccaggatctgctggtgacctttaaaaccgcgcatgcgaaaaaacaggaagtggtggtgctgggcagccaggaaggcgcgatgcataccgcgctgaccggcgcgaccgaaattcagaccagcggcaccaccaccatttttgcgggccatctgaaatgccgcctgaaaatggataaactgaccctgaaaggcgtgagctatgtgatgtgcaccggcagctttaaactggaaaaagaagtggcggaaacccagcatggcaccgtgctggtgcaggtgaaatatgaaggcaccgatgcgccgtgcaaaattccgtttagcagccaggatgaaaaaggcgtgacccagaacggccgcctgattaccgcgaacccgattgtgaccgataaagaaaaaccggtgaacattgaagcggaaccgccgtttggcgaaagctatctggtggtgggcgcgggcgaaaaagcgctgaaactgagctggtttaaaaaaggcagc

SEQ ID NO: 31 - RNA, 1188 nt, Synthetic construct
  source 1..1188: mol_type = other RNA
atgcgctgcgtgtgcattggcaaccgcgattttgtggaaggcctgagcggcgcgacctgggtggatgtggtgctggaacatggcagctgcgtgaccaccatggcgaaagataaaccgaccctggatattgaactgctgaaaaccgaagtgaccaacccggcggtgctgcgcaaactgtgcattgaagcgaaaattagcaacaccaccaccgatagccgctgcccgacccagggcgaagcgaccctggtggaagaacaggataccaactttgtgtgccgccgcacctttgtggatcgcggctggtgcaacggctgcggcctgtttggcaaaggcagcctgattacctgcgcgaaatttaaatgcgtgaccaaactggaaggcaaaattgtgcagtatgaaaacctgaaatatagcgtgattgtgaccgtgcataccggcgatcagcatcaggtgggcaacgaaaccaccgaacatggcaccaccgcgaccattaccccgcaggcgccgaccagcgaaattcagctgaccgattatggcgcgctgaccctggattgcagcccgcgcaccggcctggattttaacgaaatggtgctgctgaccatgaaagaaaaaagctggctggtgcataaacagtggtttctggatctgccgctgccgtggaccagcggcgcgagcaccagccaggaaacctggaaccgccaggatctgctggtgacctttaaaaccgcgcatgcgaaaaaacaggaagtggtggtgctgggcagccaggaaggcgcgatgcataccgcgctgaccggcgcgaccgaaattcagaccagcggcaccaccaccatttttgcgggccatctgaaatgccgcctgaaaatggataaactgaccctgaaaggcgtgagctatgtgatgtgcaccggcagctttaaactggaaaaagaagtggcggaaacccagcatggcaccgtgctggtgcaggtgaaatatgaaggcaccgatgcgccgtgcaaaattccgtttagcagccaggatgaaaaaggcgtgacccagaacggccgcctgattaccgcgaacccgattgtgaccgataaagaaaaaccggtgaacattgaagcggaaccgccgtttggcgaaagctatctggtggtgggcgcgggcgaaaaagcgctgaaactgagctggtttaaaaaaggcagc

SEQ ID NO: 32 - protein, 396 aa, Dengue virus
MRCVGVGNRDFVEGVSGGAWVDLVLEHGGCVTTMAQGKPTLDFELIKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDISNHGVTATITPRSPSVEVKLPDYGELTLDCEPRSGIDFNEMILMKMKKKTWLVHKQWFLDLPLPWAAGADTSEVHWNYKERMVTFKVPHAKRQDVTVLGSQEGAMHSALTGATEVDSGDGNHMYAGHLKCKVRMEKLRIKGMSYTMCSGKFSIDKEMAETQHGTTVVKVKYEGAGAPCKVPIEIRDVNKEKVVGRIISSTPFAEYTNSVTNIELEPPFGDSYIVIGVGDSALTLHWFRKGS

SEQ ID NO: 33 - protein, 396 aa, Synthetic construct
MRCVCVGNRDFVEGVSGGAWVDLVLEHGGCVTTMAQGKPTLDFELIKTTAKEVALLRTYCIEASISNITTATRCPTQGEPYLKEEQDQQYICRRDVVDRGWCNGCGLFGKGGVVTCAKFSCSGKITGNLVQIENLEYTVVVTVHNGDTHAVGNDISNHGVTATITPRSPSVEVKLPDYGELTLDCEPRSGIDFNEMILMKMKKKTWLVHKQWFLDLPLPWAAGADTSEVHWNYKERMVTFKVPHAKRQDVTVLGSQEGAMHSALTGATEVDSGDGNHMYAGHLKCKVRMEKLRIKGMSYTMCSGKFSIDKEMAETQHGTTVVKVKYEGAGAPCKVPIEIRDVNKEKVVGRIISSTPFAEYTNSVTNIELEPPFGDSYIVIGVGDSALTLHWFRKGS

SEQ ID NO: 34 - DNA, 1188 bp, Synthetic construct
  source 1..1188: mol_type = other DNA
atgcgctgcgtgtgcgtgggcaaccgcgattttgtggaaggcgtgagcggcggcgcgtgggtggatctggtgctggaacatggcggctgcgtgaccaccatggcgcagggcaaaccgaccctggattttgaactgattaaaaccaccgcgaaagaagtggcgctgctgcgcacctattgcattgaagcgagcattagcaacattaccaccgcgacccgctgcccgacccagggcgaaccgtatctgaaagaagaacaggatcagcagtatatttgccgccgcgatgtggtggatcgcggctggtgcaacggctgcggcctgtttggcaaaggcggcgtggtgacctgcgcgaaatttagctgcagcggcaaaattaccggcaacctggtgcagattgaaaacctggaatataccgtggtggtgaccgtgcataacggcgatacccatgcggtgggcaacgatattagcaaccatggcgtgaccgcgaccattaccccgcgcagcccgagcgtggaagtgaaactgccggattatggcgaactgaccctggattgcgaaccgcgcagcggcattgattttaacgaaatgattctgatgaaaatgaaaaaaaaaacctggctggtgcataaacagtggtttctggatctgccgctgccgtgggcggcgggcgcggataccagcgaagtgcattggaactataaagaacgcatggtgacctttaaagtgccgcatgcgaaacgccaggatgtgaccgtgctgggcagccaggaaggcgcgatgcatagcgcgctgaccggcgcgaccgaagtggatagcggcgatggcaaccatatgtatgcgggccatctgaaatgcaaagtgcgcatggaaaaactgcgcattaaaggcatgagctataccatgtgcagcggcaaatttagcattgataaagaaatggcggaaacccagcatggcaccaccgtggtgaaagtgaaatatgaaggcgcgggcgcgccgtgcaaagtgccgattgaaattcgcgatgtgaacaaagaaaaagtggtgggccgcattattagcagcaccccgtttgcggaatataccaacagcgtgaccaacattgaactggaaccgccgtttggcgatagctatattgtgattggcgtgggcgatagcgcgctgaccctgcattggtttcgcaaaggcagc

SEQ ID NO: 35 - RNA, 1188 nt, Synthetic construct
  source 1..1188: mol_type = other RNA
atgcgctgcgtgtgcgtgggcaaccgcgattttgtggaaggcgtgagcggcggcgcgtgggtggatctggtgctggaacatggcggctgcgtgaccaccatggcgcagggcaaaccgaccctggattttgaactgattaaaaccaccgcgaaagaagtggcgctgctgcgcacctattgcattgaagcgagcattagcaacattaccaccgcgacccgctgcccgacccagggcgaaccgtatctgaaagaagaacaggatcagcagtatatttgccgccgcgatgtggtggatcgcggctggtgcaacggctgcggcctgtttggcaaaggcggcgtggtgacctgcgcgaaatttagctgcagcggcaaaattaccggcaacctggtgcagattgaaaacctggaatataccgtggtggtgaccgtgcataacggcgatacccatgcggtgggcaacgatattagcaaccatggcgtgaccgcgaccattaccccgcgcagcccgagcgtggaagtgaaactgccggattatggcgaactgaccctggattgcgaaccgcgcagcggcattgattttaacgaaatgattctgatgaaaatgaaaaaaaaaacctggctggtgcataaacagtggtttctggatctgccgctgccgtgggcggcgggcgcggataccagcgaagtgcattggaactataaagaacgcatggtgacctttaaagtgccgcatgcgaaacgccaggatgtgaccgtgctgggcagccaggaaggcgcgatgcatagcgcgctgaccggcgcgaccgaagtggatagcggcgatggcaaccatatgtatgcgggccatctgaaatgcaaagtgcgcatggaaaaactgcgcattaaaggcatgagctataccatgtgcagcggcaaatttagcattgataaagaaatggcggaaacccagcatggcaccaccgtggtgaaagtgaaatatgaaggcgcgggcgcgccgtgcaaagtgccgattgaaattcgcgatgtgaacaaagaaaaagtggtgggccgcattattagcagcaccccgtttgcggaatataccaacagcgtgaccaacattgaactggaaccgccgtttggcgatagctatattgtgattggcgtgggcgatagcgcgctgaccctgcattggtttcgcaaaggcagc

SEQ ID NO: 36 - protein, 394 aa, Yellow fever virus
AHCIGITDRDFIEGVHGGTWVSATLEQDKCVTVMAPDKPSLDISLETVAIDGPAEARKVCYSAVLTNVKINDKCPSTGEAHLEEENEGDNACKRTYSDRGWGNGCGLFGKGSIVACAKFTCAKSMSLFEVDQTKIQYVIRAQLHVGAKQENWNTDIKTLKFDALSGSQEAEFTGYGKATLECQVQTALDFSNSYIAEMEKESWIVDKQWAQDLTLPWQSGSGGVWREMHHLVEFEPPHAATIKVLALGNQEGSLKTALTGAMRVTKDTNNSKLYKLHGGHVACRVKLSALTLKGTSYKMCTDKMSFVKNPTDTGHGTAVMQVKVPKGAPCRIPVMVADDLTAAVNKGILVTVNPIASTNDDEVLIEVNPPFGDSYIIVGTGDSRLTYQWHKEGS

SEQ ID NO: 37 - protein, 394 aa, Synthetic construct
AHCICITDRDFIEGVHGGTWVSATLEQDKCVTVMAPDKPSLDISLETVAIDGPAEARKVCYSAVLTNVKINDKCPSTGEAHLEEENEGDNACKRTYSDRGWCNGCGLFGKGSIVACAKFTCAKSMSLFEVDQTKIQYVIRAQLHVGAKQENWNTDIKTLKFDALSGSQEAEFTGYGKATLECQVQTALDFSNSYIAEMEKESWIVDKQWAQDLTLPWQSGSGGVWREMHHLVEFEPPHAATIKVLALGNQEGSLKTALTGAMRVTKDTNNSKLYKLHGGHVACRVKLSALTLKGTSYKMCTDKMSFVKNPTDTGHGTAVMQVKVPKGAPCRIPVMVADDLTAAVNKGILVTVNPIASTNDDEVLIEVNPPFGDSYIIVGTGDSRLTYQWHKEGS

SEQ ID NO: 38 - DNA, 1182 bp, Synthetic construct
  source 1..1182: mol_type = other DNA
gcgcattgcatttgcattaccgatcgcgattttattgaaggcgtgcatggcggcacctgggtgagcgcgaccctggaacaggataaatgcgtgaccgtgatggcgccggataaaccgagcctggatattagcctggaaaccgtggcgattgatggcccggcggaagcgcgcaaagtgtgctatagcgcggtgctgaccaacgtgaaaattaacgataaatgcccgagcaccggcgaagcgcatctggaagaagaaaacgaaggcgataacgcgtgcaaacgcacctatagcgatcgcggctggtgcaacggctgcggcctgtttggcaaaggcagcattgtggcgtgcgcgaaatttacctgcgcgaaaagcatgagcctgtttgaagtggatcagaccaaaattcagtatgtgattcgcgcgcagctgcatgtgggcgcgaaacaggaaaactggaacaccgatattaaaaccctgaaatttgatgcgctgagcggcagccaggaagcggaatttaccggctatggcaaagcgaccctggaatgccaggtgcagaccgcgctggattttagcaacagctatattgcggaaatggaaaaagaaagctggattgtggataaacagtgggcgcaggatctgaccctgccgtggcagagcggcagcggcggcgtgtggcgcgaaatgcatcatctggtggaatttgaaccgccgcatgcggcgaccattaaagtgctggcgctgggcaaccaggaaggcagcctgaaaaccgcgctgaccggcgcgatgcgcgtgaccaaagataccaacaacagcaaactgtataaactgcatggcggccatgtggcgtgccgcgtgaaactgagcgcgctgaccctgaaaggcaccagctataaaatgtgcaccgataaaatgagctttgtgaaaaacccgaccgataccggccatggcaccgcggtgatgcaggtgaaagtgccgaaaggcgcgccgtgccgcattccggtgatggtggcggatgatctgaccgcggcggtgaacaaaggcattctggtgaccgtgaacccgattgcgagcaccaacgatgatgaagtgctgattgaagtgaacccgccgtttggcgatagctatattattgtgggcaccggcgatagccgcctgacctatcagtggcataaagaaggcagc

SEQ ID NO: 39 - RNA, 1182 nt, Synthetic construct
  source 1..1182: mol_type = other RNA
gcgcattgcatttgcattaccgatcgcgattttattgaaggcgtgcatggcggcacctgggtgagcgcgaccctggaacaggataaatgcgtgaccgtgatggcgccggataaaccgagcctggatattagcctggaaaccgtggcgattgatggcccggcggaagcgcgcaaagtgtgctatagcgcggtgctgaccaacgtgaaaattaacgataaatgcccgagcaccggcgaagcgcatctggaagaagaaaacgaaggcgataacgcgtgcaaacgcacctatagcgatcgcggctggtgcaacggctgcggcctgtttggcaaaggcagcattgtggcgtgcgcgaaatttacctgcgcgaaaagcatgagcctgtttgaagtggatcagaccaaaattcagtatgtgattcgcgcgcagctgcatgtgggcgcgaaacaggaaaactggaacaccgatattaaaaccctgaaatttgatgcgctgagcggcagccaggaagcggaatttaccggctatggcaaagcgaccctggaatgccaggtgcagaccgcgctggattttagcaacagctatattgcggaaatggaaaaagaaagctggattgtggataaacagtgggcgcaggatctgaccctgccgtggcagagcggcagcggcggcgtgtggcgcgaaatgcatcatctggtggaatttgaaccgccgcatgcggcgaccattaaagtgctggcgctgggcaaccaggaaggcagcctgaaaaccgcgctgaccggcgcgatgcgcgtgaccaaagataccaacaacagcaaactgtataaactgcatggcggccatgtggcgtgccgcgtgaaactgagcgcgctgaccctgaaaggcaccagctataaaatgtgcaccgataaaatgagctttgtgaaaaacccgaccgataccggccatggcaccgcggtgatgcaggtgaaagtgccgaaaggcgcgccgtgccgcattccggtgatggtggcggatgatctgaccgcggcggtgaacaaaggcattctggtgaccgtgaacccgattgcgagcaccaacgatgatgaagtgctgattgaagtgaacccgccgtttggcgatagctatattattgtgggcaccggcgatagccgcctgacctatcagtggcataaagaaggcagc

SEQ ID NO: 40 - protein, 401 aa, Japanese encephalitis virus
FNCLGMGNRDFIEGASGATWVDLVLEGDSCLTIMANDKPALDVRMINIEASQLAEVRSYCYHASVTDISTVARCPTTGEAHNKKRADSSYVCKQGFTDRGWGNGCGLFGKGSIDTCAKFSCTNKAIGRTIQPENIKYEVGVFVHGTTTSENHGNYSAQVGASQAAKFTVTPNAPSITLKLGDYGEVTLDCEPRSGLNTEAFYVMTVGSKSFLVHREWFHDLSLPWTSPSSTAWRNRELLMEFEEAHATKQSVVALGSQEGGLHQALAGAIVVEYSSSVKLTSGHLKCRLKMDKLALKGTTYGMCTEKFSFAKNPADTGHGTVVIELTYSGSDGPCKIPIVSVASLNDMTPVGRLVTVNPFVVTSSSNSKVLVEMEPPFGDSYIVVGRGDKQINHHWYKAGS

SEQ ID NO: 41 - protein, 401 aa, Synthetic construct
FNCLCMGNRDFIEGASGATWVDLVLEGDSCLTIMANDKPALDVRMINIEASQLAEVRSYCYHASVTDISTVARCPTTGEAHNKKRADSSYVCKQGFTDRGWCNGCGLFGKGSIDTCAKFSCTNKAIGRTIQPENIKYEVGVFVHGTTTSENHGNYSAQVGASQAAKFTVTPNAPSITLKLGDYGEVTLDCEPRSGLNTEAFYVMTVGSKSFLVHREWFHDLSLPWTSPSSTAWRNRELLMEFEEAHATKQSVVALGSQEGGLHQALAGAIVVEYSSSVKLTSGHLKCRLKMDKLALKGTTYGMCTEKFSFAKNPADTGHGTVVIELTYSGSDGPCKIPIVSVASLNDMTPVGRLVTVNPFVVTSSSNSKVLVEMEPPFGDSYIVVGRGDKQINHHWYKAGS

SEQ ID NO: 42 - DNA, 1203 bp, synthetic construct
  source 1..1203: mol_type = other DNA
tttaactgcctgtgcatgggcaaccgcgattttattgaaggcgcgagcggcgcgacctgggtggatctggtgctggaaggcgatagctgcctgaccattatggcgaacgataaaccggcgctggatgtgcgcatgattaacattgaagcgagccagctggcggaagtgcgcagctattgctatcatgcgagcgtgaccgatattagcaccgtggcgcgctgcccgaccaccggcgaagcgcataacaaaaaacgcgcggatagcagctatgtgtgcaaacagggctttaccgatcgcggctggtgcaacggctgcggcctgtttggcaaaggcagcattgatacctgcgcgaaatttagctgcaccaacaaagcgattggccgcaccattcagccggaaaacattaaatatgaagtgggcgtgtttgtgcatggcaccaccaccagcgaaaaccatggcaactatagcgcgcaggtgggcgcgagccaggcggcgaaatttaccgtgaccccgaacgcgccgagcattaccctgaaactgggcgattatggcgaagtgaccctggattgcgaaccgcgcagcggcctgaacaccgaagcgttttatgtgatgaccgtgggcagcaaaagctttctggtgcatcgcgaatggtttcatgatctgagcctgccgtggaccagcccgagcagcaccgcgtggcgcaaccgcgaactgctgatggaatttgaagaagcgcatgcgaccaaacagagcgtggtggcgctgggcagccaggaaggcggcctgcatcaggcgctggcgggcgcgattgtggtggaatatagcagcagcgtgaaactgaccagcggccatctgaaatgccgcctgaaaatggataaactggcgctgaaaggcaccacctatggcatgtgcaccgaaaaatttagctttgcgaaaaacccggcggataccggccatggcaccgtggtgattgaactgacctatagcggcagcgatggcccgtgcaaaattccgattgtgagcgtggcgagcctgaacgatatgaccccggtgggccgcctggtgaccgtgaacccgtttgtggtgaccagcagcagcaacagcaaagtgctggtggaaatggaaccgccgtttggcgatagctatattgtggtgggccgcggcgataaacagattaaccatcattggtataaagcgggcagc

SEQ ID NO: 43 - RNA, 1203 nt, Synthetic construct
  source 1..1203: mol_type = other RNA
tttaactgcctgtgcatgggcaaccgcgattttattgaaggcgcgagcggcgcgacctgggtggatctggtgctggaaggcgatagctgcctgaccattatggcgaacgataaaccggcgctggatgtgcgcatgattaacattgaagcgagccagctggcggaagtgcgcagctattgctatcatgcgagcgtgaccgatattagcaccgtggcgcgctgcccgaccaccggcgaagcgcataacaaaaaacgcgcggatagcagctatgtgtgcaaacagggctttaccgatcgcggctggtgcaacggctgcggcctgtttggcaaaggcagcattgatacctgcgcgaaatttagctgcaccaacaaagcgattggccgcaccattcagccggaaaacattaaatatgaagtgggcgtgtttgtgcatggcaccaccaccagcgaaaaccatggcaactatagcgcgcaggtgggcgcgagccaggcggcgaaatttaccgtgaccccgaacgcgccgagcattaccctgaaactgggcgattatggcgaagtgaccctggattgcgaaccgcgcagcggcctgaacaccgaagcgttttatgtgatgaccgtgggcagcaaaagctttctggtgcatcgcgaatggtttcatgatctgagcctgccgtggaccagcccgagcagcaccgcgtggcgcaaccgcgaactgctgatggaatttgaagaagcgcatgcgaccaaacagagcgtggtggcgctgggcagccaggaaggcggcctgcatcaggcgctggcgggcgcgattgtggtggaatatagcagcagcgtgaaactgaccagcggccatctgaaatgccgcctgaaaatggataaactggcgctgaaaggcaccacctatggcatgtgcaccgaaaaatttagctttgcgaaaaacccggcggataccggccatggcaccgtggtgattgaactgacctatagcggcagcgatggcccgtgcaaaattccgattgtgagcgtggcgagcctgaacgatatgaccccggtgggccgcctggtgaccgtgaacccgtttgtggtgaccagcagcagcaacagcaaagtgctggtggaaatggaaccgccgtttggcgatagctatattgtggtgggccgcggcgataaacagattaaccatcattggtataaagcgggcagc

SEQ ID NO: 44 - protein, 402 aa, Saint Louis encephalitis virus
FNCLGTSNRDFVEGASGATWIDLVLEGGSCVTVMAPEKPTLDFKVMKMEATELATVREYCYEATLDTLSTVARCPTTGEAHNTKRSDPTFVCKRDVVDRGWGNGCGLFGKGSIDTCAKFTCKNKATGKTILRENIKYEVAIFVHGSTDSTSHGNYSEQIGKNQAARFTISPQAPSFTANMGEYGTVTIDCEARSGINTEDYYVFTVKEKSWLVNRDWFHDLNLPWTSPATTDWRNRETLVEFEEPHATKQTVVALGSQEGALHTALAGAIPATVSSSTLTLQSGHLKCRAKLDKVKIKGTTYGMCDSAFTFSKNPTDTGHGTVIVELQYTGSNGPCRVPISVTANLMDLTPVGRLVTVNPFISTGGANNKVMIEVEPPFGDSYIVVGRGTTQINYHWHKEGS

SEQ ID NO: 45 - protein, 402 aa, Synthetic construct
FNCLCTSNRDFVEGASGATWIDLVLEGGSCVTVMAPEKPTLDFKVMKMEATELATVREYCYEATLDTLSTVARCPTTGEAHNTKRSDPTFVCKRDVVDRGWCNGCGLFGKGSIDTCAKFTCKNKATGKTILRENIKYEVAIFVHGSTDSTSHGNYSEQIGKNQAARFTISPQAPSFTANMGEYGTVTIDCEARSGINTEDYYVFTVKEKSWLVNRDWFHDLNLPWTSPATTDWRNRETLVEFEEPHATKQTVVALGSQEGALHTALAGAIPATVSSSTLTLQSGHLKCRAKLDKVKIKGTTYGMCDSAFTFSKNPTDTGHGTVIVELQYTGSNGPCRVPISVTANLMDLTPVGRLVTVNPFISTGGANNKVMIEVEPPFGDSYIVVGRGTTQINYHWHKEGS

SEQ ID NO: 46 - DNA, 1206 bp, synthetic construct
  source 1..1206: mol_type = other DNA
tttaactgcctgtgcaccagcaaccgcgattttgtggaaggcgcgagcggcgcgacctggattgatctggtgctggaaggcggcagctgcgtgaccgtgatggcgccggaaaaaccgaccctggattttaaagtgatgaaaatggaagcgaccgaactggcgaccgtgcgcgaatattgctatgaagcgaccctggataccctgagcaccgtggcgcgctgcccgaccaccggcgaagcgcataacaccaaacgcagcgatccgacctttgtgtgcaaacgcgatgtggtggatcgcggctggtgcaacggctgcggcctgtttggcaaaggcagcattgatacctgcgcgaaatttacctgcaaaaacaaagcgaccggcaaaaccattctgcgcgaaaacattaaatatgaagtggcgatttttgtgcatggcagcaccgatagcaccagccatggcaactatagcgaacagattggcaaaaaccaggcggcgcgctttaccattagcccgcaggcgccgagctttaccgcgaacatgggcgaatatggcaccgtgaccattgattgcgaagcgcgcagcggcattaacaccgaagattattatgtgtttaccgtgaaagaaaaaagctggctggtgaaccgcgattggtttcatgatctgaacctgccgtggaccagcccggcgaccaccgattggcgcaaccgcgaaaccctggtggaatttgaagaaccgcatgcgaccaaacagaccgtggtggcgctgggcagccaggaaggcgcgctgcataccgcgctggcgggcgcgattccggcgaccgtgagcagcagcaccctgaccctgcagagcggccatctgaaatgccgcgcgaaactggataaagtgaaaattaaaggcaccacctatggcatgtgcgatagcgcgtttacctttagcaaaaacccgaccgataccggccatggcaccgtgattgtggaactgcagtataccggcagcaacggcccgtgccgcgtgccgattagcgtgaccgcgaacctgatggatctgaccccggtgggccgcctggtgaccgtgaacccgtttattagcaccggcggcgcgaacaacaaagtgatgattgaagtggaaccgccgtttggcgatagctatattgtggtgggccgcggcaccacccagattaactatcattggcataaagaaggcagc

SEQ ID NO: 47 - RNA, 1206 nt, Synthetic construct
  source 1..1206: mol_type = other RNA
tttaactgcctgtgcaccagcaaccgcgattttgtggaaggcgcgagcggcgcgacctggattgatctggtgctggaaggcggcagctgcgtgaccgtgatggcgccggaaaaaccgaccctggattttaaagtgatgaaaatggaagcgaccgaactggcgaccgtgcgcgaatattgctatgaagcgaccctggataccctgagcaccgtggcgcgctgcccgaccaccggcgaagcgcataacaccaaacgcagcgatccgacctttgtgtgcaaacgcgatgtggtggatcgcggctggtgcaacggctgcggcctgtttggcaaaggcagcattgatacctgcgcgaaatttacctgcaaaaacaaagcgaccggcaaaaccattctgcgcgaaaacattaaatatgaagtggcgatttttgtgcatggcagcaccgatagcaccagccatggcaactatagcgaacagattggcaaaaaccaggcggcgcgctttaccattagcccgcaggcgccgagctttaccgcgaacatgggcgaatatggcaccgtgaccattgattgcgaagcgcgcagcggcattaacaccgaagattattatgtgtttaccgtgaaagaaaaaagctggctggtgaaccgcgattggtttcatgatctgaacctgccgtggaccagcccggcgaccaccgattggcgcaaccgcgaaaccctggtggaatttgaagaaccgcatgcgaccaaacagaccgtggtggcgctgggcagccaggaaggcgcgctgcataccgcgctggcgggcgcgattccggcgaccgtgagcagcagcaccctgaccctgcagagcggccatctgaaatgccgcgcgaaactggataaagtgaaaattaaaggcaccacctatggcatgtgcgatagcgcgtttacctttagcaaaaacccgaccgataccggccatggcaccgtgattgtggaactgcagtataccggcagcaacggcccgtgccgcgtgccgattagcgtgaccgcgaacctgatggatctgaccccggtgggccgcctggtgaccgtgaacccgtttattagcaccggcggcgcgaacaacaaagtgatgattgaagtggaaccgccgtttggcgatagctatattgtggtgggccgcggcaccacccagattaactatcattggcataaagaaggcagc

SEQ ID NO: 48 - protein, 397 aa, Tick-borne encephalitis virus
SRCTHLENRDFVTGTQGTTRVTLVLELGGCVTITAEGKPSMDVWLDAIYQENPAKTREYCLHAKLSDTKVAARCPTMGPATLAKEHQGGTVCKRDQSDRGWGNHCGLFGKGSIVACVKAACEAKKKATGHVYDANKIVYTVKVEPHTGDYVAANETHSGRKTASFTVSSEKTILTMGEYGDVSLLCRVASGVDLAQTVILELDKTVEHLPTAWQVHRDWFNDLALPWKHEGAQNWNNAERLVEFGAPHAVKMDVYNLGDQTGVLLKALAGVPVAHIEGTKYHLKSGHVTCEVGLEKLKMKGLTYTMCDKTKFTWKRAPTDSGHDTVVMEVTFSGTKPCRIPVRAVAHGSPDVNVAMLITPNPTIETNGGGFIEMQLPPGDNIIYVGELSHQWFQKGS

SEQ ID NO: 49 - protein, 397 aa, Synthetic construct
SRCTCLENRDFVTGTQGTTRVTLVLELGGCVTITAEGKPSMDVWLDAIYQENPAKTREYCLHAKLSDTKVAARCPTMGPATLAKEHQGGTVCKRDQSDRGWCNHCGLFGKGSIVACVKAACEAKKKATGHVYDANKIVYTVKVEPHTGDYVAANETHSGRKTASFTVSSEKTILTMGEYGDVSLLCRVASGVDLAQTVILELDKTVEHLPTAWQVHRDWFNDLALPWKHEGAQNWNNAERLVEFGAPHAVKMDVYNLGDQTGVLLKALAGVPVAHIEGTKYHLKSGHVTCEVGLEKLKMKGLTYTMCDKTKFTWKRAPTDSGHDTVVMEVTFSGTKPCRIPVRAVAHGSPDVNVAMLITPNPTIETNGGGFIEMQLPPGDNIIYVGELSHQWFQKGS

SEQ ID NO: 50 - DNA, 1191 bp, Synthetic construct
  source 1..1191: mol_type = other DNA
agccgctgcacctgcctggaaaaccgcgattttgtgaccggcacccagggcaccacccgcgtgaccctggtgctggaactgggcggctgcgtgaccattaccgcggaaggcaaaccgagcatggatgtgtggctggatgcgatttatcaggaaaacccggcgaaaacccgcgaatattgcctgcatgcgaaactgagcgataccaaagtggcggcgcgctgcccgaccatgggcccggcgaccctggcgaaagaacatcagggcggcaccgtgtgcaaacgcgatcagagcgatcgcggctggtgcaaccattgcggcctgtttggcaaaggcagcattgtggcgtgcgtgaaagcggcgtgcgaagcgaaaaaaaaagcgaccggccatgtgtatgatgcgaacaaaattgtgtataccgtgaaagtggaaccgcataccggcgattatgtggcggcgaacgaaacccatagcggccgcaaaaccgcgagctttaccgtgagcagcgaaaaaaccattctgaccatgggcgaatatggcgatgtgagcctgctgtgccgcgtggcgagcggcgtggatctggcgcagaccgtgattctggaactggataaaaccgtggaacatctgccgaccgcgtggcaggtgcatcgcgattggtttaacgatctggcgctgccgtggaaacatgaaggcgcgcagaactggaacaacgcggaacgcctggtggaatttggcgcgccgcatgcggtgaaaatggatgtgtataacctgggcgatcagaccggcgtgctgctgaaagcgctggcgggcgtgccggtggcgcatattgaaggcaccaaatatcatctgaaaagcggccatgtgacctgcgaagtgggcctggaaaaactgaaaatgaaaggcctgacctataccatgtgcgataaaaccaaatttacctggaaacgcgcgccgaccgatagcggccatgataccgtggtgatggaagtgacctttagcggcaccaaaccgtgccgcattccggtgcgcgcggtggcgcatggcagcccggatgtgaacgtggcgatgctgattaccccgaacccgaccattgaaaccaacggcggcggctttattgaaatgcagctgccgccgggcgataacattatttatgtgggcgaactgagccatcagtggtttcagaaaggcagc

SEQ ID NO: 51 - RNA, 1191 nt, Synthetic construct
  source 1..1191: mol_type = other RNA
agccgctgcacctgcctggaaaaccgcgattttgtgaccggcacccagggcaccacccgcgtgaccctggtgctggaactgggcggctgcgtgaccattaccgcggaaggcaaaccgagcatggatgtgtggctggatgcgatttatcaggaaaacccggcgaaaacccgcgaatattgcctgcatgcgaaactgagcgataccaaagtggcggcgcgctgcccgaccatgggcccggcgaccctggcgaaagaacatcagggcggcaccgtgtgcaaacgcgatcagagcgatcgcggctggtgcaaccattgcggcctgtttggcaaaggcagcattgtggcgtgcgtgaaagcggcgtgcgaagcgaaaaaaaaagcgaccggccatgtgtatgatgcgaacaaaattgtgtataccgtgaaagtggaaccgcataccggcgattatgtggcggcgaacgaaacccatagcggccgcaaaaccgcgagctttaccgtgagcagcgaaaaaaccattctgaccatgggcgaatatggcgatgtgagcctgctgtgccgcgtggcgagcggcgtggatctggcgcagaccgtgattctggaactggataaaaccgtggaacatctgccgaccgcgtggcaggtgcatcgcgattggtttaacgatctggcgctgccgtggaaacatgaaggcgcgcagaactggaacaacgcggaacgcctggtggaatttggcgcgccgcatgcggtgaaaatggatgtgtataacctgggcgatcagaccggcgtgctgctgaaagcgctggcgggcgtgccggtggcgcatattgaaggcaccaaatatcatctgaaaagcggccatgtgacctgcgaagtgggcctggaaaaactgaaaatgaaaggcctgacctataccatgtgcgataaaaccaaatttacctggaaacgcgcgccgaccgatagcggccatgataccgtggtgatggaagtgacctttagcggcaccaaaccgtgccgcattccggtgcgcgcggtggcgcatggcagcccggatgtgaacgtggcgatgctgattaccccgaacccgaccattgaaaccaacggcggcggctttattgaaatgcagctgccgccgggcgataacattatttatgtgggcgaactgagccatcagtggtttcagaaaggcagc

SEQ ID NO: 52 - protein, 402 aa, Ilheus virus
MNCLGISNRDFVEGLSGGTWVDIVLEGGSCVTVMAKDKPTLDIKLIRMEAKDLATVRSYCYQATVTDSSTEARCPTMGEAHNSKSLDASYVCKSSYVDRGWGNGCGLFGKGSIQTCVKFSCPGKATGKSIQRENLNYDVAVYVHGPTSAAAHGNYTAQLTGKYAAKFSITPSAPTYTANLGEYGEATMECEPRAALDIDNYYVMSLNNKHWLVNRDWFHDLDLPWTGPATESWKNRESLIEFEEPHATRQTVVALGNQEGALHTALAGAIPVEVSSTTLTLNSGHLKCRLKLDKLKIKGTTYAMCKGTFAFAQTPVDTGHGTIVAELTYTGTDGPCKIPISMTADLRDMTPIGRLVTVNPIIPSSANSQKILVELEPPFGSSFILVGQESNQIKYQWHKTGS

SEQ ID NO: 53 - protein, 402 aa, Synthetic construct
MNCLCISNRDFVEGLSGGTWVDIVLEGGSCVTVMAKDKPTLDIKLIRMEAKDLATVRSYCYQATVTDSSTEARCPTMGEAHNSKSLDASYVCKSSYVDRGWCNGCGLFGKGSIQTCVKFSCPGKATGKSIQRENLNYDVAVYVHGPTSAAAHGNYTAQLTGKYAAKFSITPSAPTYTANLGEYGEATMECEPRAALDIDNYYVMSLNNKHWLVNRDWFHDLDLPWTGPATESWKNRESLIEFEEPHATRQTVVALGNQEGALHTALAGAIPVEVSSTTLTLNSGHLKCRLKLDKLKIKGTTYAMCKGTFAFAQTPVDTGHGTIVAELTYTGTDGPCKIPISMTADLRDMTPIGRLVTVNPIIPSSANSQKILVELEPPFGSSFILVGQESNQIKYQWHKTGS

SEQ ID NO: 54 - DNA, 1206 bp, Synthetic construct
  source 1..1206: mol_type = other DNA
atgaactgcctgtgcattagcaaccgcgattttgtggaaggcctgagcggcggcacctgggtggatattgtgctggaaggcggcagctgcgtgaccgtgatggcgaaagataaaccgaccctggatattaaactgattcgcatggaagcgaaagatctggcgaccgtgcgcagctattgctatcaggcgaccgtgaccgatagcagcaccgaagcgcgctgcccgaccatgggcgaagcgcataacagcaaaagcctggatgcgagctatgtgtgcaaaagcagctatgtggatcgcggctggtgcaacggctgcggcctgtttggcaaaggcagcattcagacctgcgtgaaatttagctgcccgggcaaagcgaccggcaaaagcattcagcgcgaaaacctgaactatgatgtggcggtgtatgtgcatggcccgaccagcgcggcggcgcatggcaactataccgcgcagctgaccggcaaatatgcggcgaaatttagcattaccccgagcgcgccgacctataccgcgaacctgggcgaatatggcgaagcgaccatggaatgcgaaccgcgcgcggcgctggatattgataactattatgtgatgagcctgaacaacaaacattggctggtgaaccgcgattggtttcatgatctggatctgccgtggaccggcccggcgaccgaaagctggaaaaaccgcgaaagcctgattgaatttgaagaaccgcatgcgacccgccagaccgtggtggcgctgggcaaccaggaaggcgcgctgcataccgcgctggcgggcgcgattccggtggaagtgagcagcaccaccctgaccctgaacagcggccatctgaaatgccgcctgaaactggataaactgaaaattaaaggcaccacctatgcgatgtgcaaaggcacctttgcgtttgcgcagaccccggtggataccggccatggcaccattgtggcggaactgacctataccggcaccgatggcccgtgcaaaattccgattagcatgaccgcggatctgcgcgatatgaccccgattggccgcctggtgaccgtgaacccgattattccgagcagcgcgaacagccagaaaattctggtggaactggaaccgccgtttggcagcagctttattctggtgggccaggaaagcaaccagattaaatatcagtggcataaaaccggcagc

SEQ ID NO: 55 - RNA, 1206 nt, Synthetic construct
  source 1..1206: mol_type = other RNA
atgaactgcctgtgcattagcaaccgcgattttgtggaaggcctgagcggcggcacctgggtggatattgtgctggaaggcggcagctgcgtgaccgtgatggcgaaagataaaccgaccctggatattaaactgattcgcatggaagcgaaagatctggcgaccgtgcgcagctattgctatcaggcgaccgtgaccgatagcagcaccgaagcgcgctgcccgaccatgggcgaagcgcataacagcaaaagcctggatgcgagctatgtgtgcaaaagcagctatgtggatcgcggctggtgcaacggctgcggcctgtttggcaaaggcagcattcagacctgcgtgaaatttagctgcccgggcaaagcgaccggcaaaagcattcagcgcgaaaacctgaactatgatgtggcggtgtatgtgcatggcccgaccagcgcggcggcgcatggcaactataccgcgcagctgaccggcaaatatgcggcgaaatttagcattaccccgagcgcgccgacctataccgcgaacctgggcgaatatggcgaagcgaccatggaatgcgaaccgcgcgcggcgctggatattgataactattatgtgatgagcctgaacaacaaacattggctggtgaaccgcgattggtttcatgatctggatctgccgtggaccggcccggcgaccgaaagctggaaaaaccgcgaaagcctgattgaatttgaagaaccgcatgcgacccgccagaccgtggtggcgctgggcaaccaggaaggcgcgctgcataccgcgctggcgggcgcgattccggtggaagtgagcagcaccaccctgaccctgaacagcggccatctgaaatgccgcctgaaactggataaactgaaaattaaaggcaccacctatgcgatgtgcaaaggcacctttgcgtttgcgcagaccccggtggataccggccatggcaccattgtggcggaactgacctataccggcaccgatggcccgtgcaaaattccgattagcatgaccgcggatctgcgcgatatgaccccgattggccgcctggtgaccgtgaacccgattattccgagcagcgcgaacagccagaaaattctggtggaactggaaccgccgtttggcagcagctttattctggtgggccaggaaagcaaccagattaaatatcagtggcataaaaccggcagc

SEQ ID NO: 56 - protein, 391 aa, Wesselsbron virus
THCLGIPKRDFIRGLDGNTWVSVVLEQGSCVTLIADNKPSVDIWLSSIVVDTPTLVRKVCYASSVTGSKASGACPTMGDAHMSEEGNEEWECKRSYSDRGWGNSCGLFGKGSIVACAKFSCTHEMEVYQIDATKIEYTISAQVHSGAKKDDWVNHTKLVTFVPTTGTSTVAFTGYGNFGLECHVQMMVDLSNSYLVKVGTDAWLVNKQWVHDITLPWQSGTGGHWRDKHFMVDFEQPHAVTMKALVLGSQEGALRTALSGAMVVELNSNKYSLKGGHVTCKAYMNNLILKGSTYSMCKRGMSFAKQPVETDHGTAVMQIKVTTGAPCRIPVIAADSMAGTENRGSVITTNPIAASNNDEVLVEISPPFGESYIIVGNGDDKLTYHWQRSGS

SEQ ID NO: 57 - protein, 391 aa, Synthetic construct
THCLCIPKRDFIRGLDGNTWVSVVLEQGSCVTLIADNKPSVDIWLSSIVVDTPTLVRKVCYASSVTGSKASGACPTMGDAHMSEEGNEEWECKRSYSDRGWCNSCGLFGKGSIVACAKFSCTHEMEVYQIDATKIEYTISAQVHSGAKKDDWVNHTKLVTFVPTTGTSTVAFTGYGNFGLECHVQMMVDLSNSYLVKVGTDAWLVNKQWVHDITLPWQSGTGGHWRDKHFMVDFEQPHAVTMKALVLGSQEGALRTALSGAMVVELNSNKYSLKGGHVTCKAYMNNLILKGSTYSMCKRGMSFAKQPVETDHGTAVMQIKVTTGAPCRIPVIAADSMAGTENRGSVITTNPIAASNNDEVLVEISPPFGESYIIVGNGDDKLTYHWQRSGS

SEQ ID NO: 58 - DNA, 1173 bp, Synthetic construct
  source 1..1173: mol_type = other DNA
acccattgcctgtgcattccgaaacgcgattttattcgcggcctggatggcaacacctgggtgagcgtggtgctggaacagggcagctgcgtgaccctgattgcggataacaaaccgagcgtggatatttggctgagcagcattgtggtggataccccgaccctggtgcgcaaagtgtgctatgcgagcagcgtgaccggcagcaaagcgagcggcgcgtgcccgaccatgggcgatgcgcatatgagcgaagaaggcaacgaagaatgggaatgcaaacgcagctatagcgatcgcggctggtgcaacagctgcggcctgtttggcaaaggcagcattgtggcgtgcgcgaaatttagctgcacccatgaaatggaagtgtatcagattgatgcgaccaaaattgaatataccattagcgcgcaggtgcatagcggcgcgaaaaaagatgattgggtgaaccataccaaactggtgacctttgtgccgaccaccggcaccagcaccgtggcgtttaccggctatggcaactttggcctggaatgccatgtgcagatgatggtggatctgagcaacagctatctggtgaaagtgggcaccgatgcgtggctggtgaacaaacagtgggtgcatgatattaccctgccgtggcagagcggcaccggcggccattggcgcgataaacattttatggtggattttgaacagccgcatgcggtgaccatgaaagcgctggtgctgggcagccaggaaggcgcgctgcgcaccgcgctgagcggcgcgatggtggtggaactgaacagcaacaaatatagcctgaaaggcggccatgtgacctgcaaagcgtatatgaacaacctgattctgaaaggcagcacctatagcatgtgcaaacgcggcatgagctttgcgaaacagccggtggaaaccgatcatggcaccgcggtgatgcagattaaagtgaccaccggcgcgccgtgccgcattccggtgattgcggcggatagcatggcgggcaccgaaaaccgcggcagcgtgattaccaccaacccgattgcggcgagcaacaacgatgaagtgctggtggaaattagcccgccgtttggcgaaagctatattattgtgggcaacggcgatgataaactgacctatcattggcagcgcagcggcagc

SEQ ID NO: 59 - RNA, 1173 nt, Synthetic construct
  source 1..1173: mol_type = other RNA
acccattgcctgtgcattccgaaacgcgattttattcgcggcctggatggcaacacctgggtgagcgtggtgctggaacagggcagctgcgtgaccctgattgcggataacaaaccgagcgtggatatttggctgagcagcattgtggtggataccccgaccctggtgcgcaaagtgtgctatgcgagcagcgtgaccggcagcaaagcgagcggcgcgtgcccgaccatgggcgatgcgcatatgagcgaagaaggcaacgaagaatgggaatgcaaacgcagctatagcgatcgcggctggtgcaacagctgcggcctgtttggcaaaggcagcattgtggcgtgcgcgaaatttagctgcacccatgaaatggaagtgtatcagattgatgcgaccaaaattgaatataccattagcgcgcaggtgcatagcggcgcgaaaaaagatgattgggtgaaccataccaaactggtgacctttgtgccgaccaccggcaccagcaccgtggcgtttaccggctatggcaactttggcctggaatgccatgtgcagatgatggtggatctgagcaacagctatctggtgaaagtgggcaccgatgcgtggctggtgaacaaacagtgggtgcatgatattaccctgccgtggcagagcggcaccggcggccattggcgcgataaacattttatggtggattttgaacagccgcatgcggtgaccatgaaagcgctggtgctgggcagccaggaaggcgcgctgcgcaccgcgctgagcggcgcgatggtggtggaactgaacagcaacaaatatagcctgaaaggcggccatgtgacctgcaaagcgtatatgaacaacctgattctgaaaggcagcacctatagcatgtgcaaacgcggcatgagctttgcgaaacagccggtggaaaccgatcatggcaccgcggtgatgcagattaaagtgaccaccggcgcgccgtgccgcattccggtgattgcggcggatagcatggcgggcaccgaaaaccgcggcagcgtgattaccaccaacccgattgcggcgagcaacaacgatgaagtgctggtggaaattagcccgccgtttggcgaaagctatattattgtgggcaacggcgatgataaactgacctatcattggcagcgcagcggcagc

SEQ ID NO: 60 - protein, 401 aa, Usutu virus
FNCLGMSNRDFLEGVSGATWVDVVLEGDSCITIMAKDKPTIDIKMMETEATNLAEVRSYCYLATVSDVSTVSNCPTTGEAHNPKRAEDTYVCKSGVTDRGWGNGCGLFGKGSIDTCANFTCSLKAMGRMIQPENVKYEVGIFIHGSTSSDTHGNYSSQLGASQAGRFTITPNSPAITVKMGDYGEISVECEPRNGLNTEAYYIMSVGTKHFLVHREWFNDLALPWTSPASSNWRNREILLEFEEPHATKQSVVALGSQEGALHQALAGAVPVSFSGSVKLTSGHLKCRVKMEKLTLKGTTYGMCTEKFSFAKNPADTGHGTVVLELQYTGSDGPCKIPISIVASLSDLTPIGRMVTANPYVASSEANAKVLVEMEPPFGDSYIVVGRGDKQINHHWHKAGS

SEQ ID NO: 61 - protein, 401 aa, Synthetic construct
FNCLCMSNRDFLEGVSGATWVDVVLEGDSCITIMAKDKPTIDIKMMETEATNLAEVRSYCYLATVSDVSTVSNCPTTGEAHNPKRAEDTYVCKSGVTDRGWCNGCGLFGKGSIDTCANFTCSLKAMGRMIQPENVKYEVGIFIHGSTSSDTHGNYSSQLGASQAGRFTITPNSPAITVKMGDYGEISVECEPRNGLNTEAYYIMSVGTKHFLVHREWFNDLALPWTSPASSNWRNREILLEFEEPHATKQSVVALGSQEGALHQALAGAVPVSFSGSVKLTSGHLKCRVKMEKLTLKGTTYGMCTEKFSFAKNPADTGHGTVVLELQYTGSDGPCKIPISIVASLSDLTPIGRMVTANPYVASSEANAKVLVEMEPPFGDSYIVVGRGDKQINHHWHKAGS

SEQ ID NO: 62 - DNA, 1203 bp, Synthetic construct
  source 1..1203: mol_type = other DNA
tttaactgcctgtgcatgagcaaccgcgattttctggaaggcgtgagcggcgcgacctgggtggatgtggtgctggaaggcgatagctgcattaccattatggcgaaagataaaccgaccattgatattaaaatgatggaaaccgaagcgaccaacctggcggaagtgcgcagctattgctatctggcgaccgtgagcgatgtgagcaccgtgagcaactgcccgaccaccggcgaagcgcataacccgaaacgcgcggaagatacctatgtgtgcaaaagcggcgtgaccgatcgcggctggtgcaacggctgcggcctgtttggcaaaggcagcattgatacctgcgcgaactttacctgcagcctgaaagcgatgggccgcatgattcagccggaaaacgtgaaatatgaagtgggcatttttattcatggcagcaccagcagcgatacccatggcaactatagcagccagctgggcgcgagccaggcgggccgctttaccattaccccgaacagcccggcgattaccgtgaaaatgggcgattatggcgaaattagcgtggaatgcgaaccgcgcaacggcctgaacaccgaagcgtattatattatgagcgtgggcaccaaacattttctggtgcatcgcgaatggtttaacgatctggcgctgccgtggaccagcccggcgagcagcaactggcgcaaccgcgaaattctgctggaatttgaagaaccgcatgcgaccaaacagagcgtggtggcgctgggcagccaggaaggcgcgctgcatcaggcgctggcgggcgcggtgccggtgagctttagcggcagcgtgaaactgaccagcggccatctgaaatgccgcgtgaaaatggaaaaactgaccctgaaaggcaccacctatggcatgtgcaccgaaaaatttagctttgcgaaaaacccggcggataccggccatggcaccgtggtgctggaactgcagtataccggcagcgatggcccgtgcaaaattccgattagcattgtggcgagcctgagcgatctgaccccgattggccgcatggtgaccgcgaacccgtatgtggcgagcagcgaagcgaacgcgaaagtgctggtggaaatggaaccgccgtttggcgatagctatattgtggtgggccgcggcgataaacagattaaccatcattggcataaagcgggcagc

SEQ ID NO: 63 - RNA, 1203 nt, Synthetic construct
  source 1..1203: mol_type = other RNA
tttaactgcctgtgcatgagcaaccgcgattttctggaaggcgtgagcggcgcgacctgggtggatgtggtgctggaaggcgatagctgcattaccattatggcgaaagataaaccgaccattgatattaaaatgatggaaaccgaagcgaccaacctggcggaagtgcgcagctattgctatctggcgaccgtgagcgatgtgagcaccgtgagcaactgcccgaccaccggcgaagcgcataacccgaaacgcgcggaagatacctatgtgtgcaaaagcggcgtgaccgatcgcggctggtgcaacggctgcggcctgtttggcaaaggcagcattgatacctgcgcgaactttacctgcagcctgaaagcgatgggccgcatgattcagccggaaaacgtgaaatatgaagtgggcatttttattcatggcagcaccagcagcgatacccatggcaactatagcagccagctgggcgcgagccaggcgggccgctttaccattaccccgaacagcccggcgattaccgtgaaaatgggcgattatggcgaaattagcgtggaatgcgaaccgcgcaacggcctgaacaccgaagcgtattatattatgagcgtgggcaccaaacattttctggtgcatcgcgaatggtttaacgatctggcgctgccgtggaccagcccggcgagcagcaactggcgcaaccgcgaaattctgctggaatttgaagaaccgcatgcgaccaaacagagcgtggtggcgctgggcagccaggaaggcgcgctgcatcaggcgctggcgggcgcggtgccggtgagctttagcggcagcgtgaaactgaccagcggccatctgaaatgccgcgtgaaaatggaaaaactgaccctgaaaggcaccacctatggcatgtgcaccgaaaaatttagctttgcgaaaaacccggcggataccggccatggcaccgtggtgctggaactgcagtataccggcagcgatggcccgtgcaaaattccgattagcattgtggcgagcctgagcgatctgaccccgattggccgcatggtgaccgcgaacccgtatgtggcgagcagcgaagcgaacgcgaaagtgctggtggaaatggaaccgccgtttggcgatagctatattgtggtgggccgcggcgataaacagattaaccatcattggcataaagcgggcagc

SEQ ID NO: 64 - protein, 398 aa, Powassan virus
TRCTHLENRDFVTGTQGTTRVSLVLELGGCVTITAEGKPSIDVWLEDIFQESPAETREYCLHAKLTNTKVEARCPTTGPATLPEEHQANMVCKRDQSDRGWGNHCGFFGKGSIVACAKFECEEAKKAVGHVYDSTKITYVVKVEPHTGDYLAANETNSNRKSAQFTVASEKVILRLGDYGDVSLTCKVASGIDVAQTVVMSLDSSKDHLPSAWQVHRDWFEDLALPWKHKDNQDWNSVEKLVEFGPPHAVKMDVFNLGDQTAVLLKSLAGVPLASVEGQKYHLKSGHVTCDVGLEKLKLKGTTYSMCDKAKFKWKRVPVDSGHDTVVMEVSYTGSDKPCRIPVRAVAHGVPAVNVAMLITPNPTIETNGGGFIEMQLPPGDNIIYVGDLSQQWFQKGS

SEQ ID NO: 65 - protein, 398 aa, Synthetic construct
TRCTCLENRDFVTGTQGTTRVSLVLELGGCVTITAEGKPSIDVWLEDIFQESPAETREYCLHAKLTNTKVEARCPTTGPATLPEEHQANMVCKRDQSDRGWCNHCGFFGKGSIVACAKFECEEAKKAVGHVYDSTKITYVVKVEPHTGDYLAANETNSNRKSAQFTVASEKVILRLGDYGDVSLTCKVASGIDVAQTVVMSLDSSKDHLPSAWQVHRDWFEDLALPWKHKDNQDWNSVEKLVEFGPPHAVKMDVFNLGDQTAVLLKSLAGVPLASVEGQKYHLKSGHVTCDVGLEKLKLKGTTYSMCDKAKFKWKRVPVDSGHDTVVMEVSYTGSDKPCRIPVRAVAHGVPAVNVAMLITPNPTIETNGGGFIEMQLPPGDNIIYVGDLSQQWFQKGS

SEQ ID NO: 66 - DNA, 1194 bp, Synthetic construct
  source 1..1194: mol_type = other DNA
acccgctgcacctgcctggaaaaccgcgattttgtgaccggcacccagggcaccacccgcgtgagcctggtgctggaactgggcggctgcgtgaccattaccgcggaaggcaaaccgagcattgatgtgtggctggaagatatttttcaggaaagcccggcggaaacccgcgaatattgcctgcatgcgaaactgaccaacaccaaagtggaagcgcgctgcccgaccaccggcccggcgaccctgccggaagaacatcaggcgaacatggtgtgcaaacgcgatcagagcgatcgcggctggtgcaaccattgcggcttttttggcaaaggcagcattgtggcgtgcgcgaaatttgaatgcgaagaagcgaaaaaagcggtgggccatgtgtatgatagcaccaaaattacctatgtggtgaaagtggaaccgcataccggcgattatctggcggcgaacgaaaccaacagcaaccgcaaaagcgcgcagtttaccgtggcgagcgaaaaagtgattctgcgcctgggcgattatggcgatgtgagcctgacctgcaaagtggcgagcggcattgatgtggcgcagaccgtggtgatgagcctggatagcagcaaagatcatctgccgagcgcgtggcaggtgcatcgcgattggtttgaagatctggcgctgccgtggaaacataaagataaccaggattggaacagcgtggaaaaactggtggaatttggcccgccgcatgcggtgaaaatggatgtgtttaacctgggcgatcagaccgcggtgctgctgaaaagcctggcgggcgtgccgctggcgagcgtggaaggccagaaatatcatctgaaaagcggccatgtgacctgcgatgtgggcctggaaaaactgaaactgaaaggcaccacctatagcatgtgcgataaagcgaaatttaaatggaaacgcgtgccggtggatagcggccatgataccgtggtgatggaagtgagctataccggcagcgataaaccgtgccgcattccggtgcgcgcggtggcgcatggcgtgccggcggtgaacgtggcgatgctgattaccccgaacccgaccattgaaaccaacggcggcggctttattgaaatgcagctgccgccgggcgataacattatttatgtgggcgatctgagccagcagtggtttcagaaaggcagc

SEQ ID NO: 67 - RNA, 1194 nt, Synthetic construct
  source 1..1194: mol_type = other RNA
acccgctgcacctgcctggaaaaccgcgattttgtgaccggcacccagggcaccacccgcgtgagcctggtgctggaactgggcggctgcgtgaccattaccgcggaaggcaaaccgagcattgatgtgtggctggaagatatttttcaggaaagcccggcggaaacccgcgaatattgcctgcatgcgaaactgaccaacaccaaagtggaagcgcgctgcccgaccaccggcccggcgaccctgccggaagaacatcaggcgaacatggtgtgcaaacgcgatcagagcgatcgcggctggtgcaaccattgcggcttttttggcaaaggcagcattgtggcgtgcgcgaaatttgaatgcgaagaagcgaaaaaagcggtgggccatgtgtatgatagcaccaaaattacctatgtggtgaaagtggaaccgcataccggcgattatctggcggcgaacgaaaccaacagcaaccgcaaaagcgcgcagtttaccgtggcgagcgaaaaagtgattctgcgcctgggcgattatggcgatgtgagcctgacctgcaaagtggcgagcggcattgatgtggcgcagaccgtggtgatgagcctggatagcagcaaagatcatctgccgagcgcgtggcaggtgcatcgcgattggtttgaagatctggcgctgccgtggaaacataaagataaccaggattggaacagcgtggaaaaactggtggaatttggcccgccgcatgcggtgaaaatggatgtgtttaacctgggcgatcagaccgcggtgctgctgaaaagcctggcgggcgtgccgctggcgagcgtggaaggccagaaatatcatctgaaaagcggccatgtgacctgcgatgtgggcctggaaaaactgaaactgaaaggcaccacctatagcatgtgcgataaagcgaaatttaaatggaaacgcgtgccggtggatagcggccatgataccgtggtgatggaagtgagctataccggcagcgataaaccgtgccgcattccggtgcgcgcggtggcgcatggcgtgccggcggtgaacgtggcgatgctgattaccccgaacccgaccattgaaaccaacggcggcggctttattgaaatgcagctgccgccgggcgataacattatttatgtgggcgatctgagccagcagtggtttcagaaaggcagc

SEQ ID NO: 68 - protein, 402 aa, Orthoflavivirus rocioense
INCLGVTNRDFVEGMSGGTWVDIVLEGDGCVTIMAKDKPTLDIRLLKMEAKDLATVRSYCYHATVTSVSSEARCPTMGEAHNPKALDSNYLCKSTYVDRGWGNGCGLFGKGSLQTCVKFGCTQKAMGMTIQRENLDYELAIYVHGPTSVAAHGNYTTQLGAKHAAKFSITPSSPSFTANLGEYGEATVDCEPRAALDIDNYYVMSMNNKHWLVNRDWFHDLDLPWTGPATDVWKYRESLVEFEEAHVTRQTVVALAAQEGELHIVLAGAIPVTVAGTTLTLTSGHLKCRMKLDKLKIKGSTYLMCKDKFAFAKNPVDTGHGTIVTEVQYAGSDGPCRIPITMTENLHDLTPIGRLVTVNPFVPSSETAQKILIELEPPFGTSFILVGTGPNQVKYQWHKSGS

SEQ ID NO: 69 - protein, 402 aa, synthetic construct
INCLCVTNRDFVEGMSGGTWVDIVLEGDGCVTIMAKDKPTLDIRLLKMEAKDLATVRSYCYHATVTSVSSEARCPTMGEAHNPKALDSNYLCKSTYVDRGWCNGCGLFGKGSLQTCVKFGCTQKAMGMTIQRENLDYELAIYVHGPTSVAAHGNYTTQLGAKHAAKFSITPSSPSFTANLGEYGEATVDCEPRAALDIDNYYVMSMNNKHWLVNRDWFHDLDLPWTGPATDVWKYRESLVEFEEAHVTRQTVVALAAQEGELHIVLAGAIPVTVAGTTLTLTSGHLKCRMKLDKLKIKGSTYLMCKDKFAFAKNPVDTGHGTIVTEVQYAGSDGPCRIPITMTENLHDLTPIGRLVTVNPFVPSSETAQKILIELEPPFGTSFILVGTGPNQVKYQWHKSGS

SEQ ID NO: 70 - DNA, 1206 bp, synthetic construct
  source 1..1206: mol_type = other DNA
attaactgcctgtgcgtgaccaaccgcgattttgtggaaggcatgagcggcggcacctgggtggatattgtgctggaaggcgatggctgcgtgaccattatggcgaaagataaaccgaccctggatattcgcctgctgaaaatggaagcgaaagatctggcgaccgtgcgcagctattgctatcatgcgaccgtgaccagcgtgagcagcgaagcgcgctgcccgaccatgggcgaagcgcataacccgaaagcgctggatagcaactatctgtgcaaaagcacctatgtggatcgcggctggtgcaacggctgcggcctgtttggcaaaggcagcctgcagacctgcgtgaaatttggctgcacccagaaagcgatgggcatgaccattcagcgcgaaaacctggattatgaactggcgatttatgtgcatggcccgaccagcgtggcggcgcatggcaactataccacccagctgggcgcgaaacatgcggcgaaatttagcattaccccgagcagcccgagctttaccgcgaacctgggcgaatatggcgaagcgaccgtggattgcgaaccgcgcgcggcgctggatattgataactattatgtgatgagcatgaacaacaaacattggctggtgaaccgcgattggtttcatgatctggatctgccgtggaccggcccggcgaccgatgtgtggaaatatcgcgaaagcctggtggaatttgaagaagcgcatgtgacccgccagaccgtggtggcgctggcggcgcaggaaggcgaactgcatattgtgctggcgggcgcgattccggtgaccgtggcgggcaccaccctgaccctgaccagcggccatctgaaatgccgcatgaaactggataaactgaaaattaaaggcagcacctatctgatgtgcaaagataaatttgcgtttgcgaaaaacccggtggataccggccatggcaccattgtgaccgaagtgcagtatgcgggcagcgatggcccgtgccgcattccgattaccatgaccgaaaacctgcatgatctgaccccgattggccgcctggtgaccgtgaacccgtttgtgccgagcagcgaaaccgcgcagaaaattctgattgaactggaaccgccgtttggcaccagctttattctggtgggcaccggcccgaaccaggtgaaatatcagtggcataaaagcggcagc

SEQ ID NO: 71 - RNA, 1206 nt, Synthetic construct
  source 1..1206: mol_type = other RNA
attaactgcctgtgcgtgaccaaccgcgattttgtggaaggcatgagcggcggcacctgggtggatattgtgctggaaggcgatggctgcgtgaccattatggcgaaagataaaccgaccctggatattcgcctgctgaaaatggaagcgaaagatctggcgaccgtgcgcagctattgctatcatgcgaccgtgaccagcgtgagcagcgaagcgcgctgcccgaccatgggcgaagcgcataacccgaaagcgctggatagcaactatctgtgcaaaagcacctatgtggatcgcggctggtgcaacggctgcggcctgtttggcaaaggcagcctgcagacctgcgtgaaatttggctgcacccagaaagcgatgggcatgaccattcagcgcgaaaacctggattatgaactggcgatttatgtgcatggcccgaccagcgtggcggcgcatggcaactataccacccagctgggcgcgaaacatgcggcgaaatttagcattaccccgagcagcccgagctttaccgcgaacctgggcgaatatggcgaagcgaccgtggattgcgaaccgcgcgcggcgctggatattgataactattatgtgatgagcatgaacaacaaacattggctggtgaaccgcgattggtttcatgatctggatctgccgtggaccggcccggcgaccgatgtgtggaaatatcgcgaaagcctggtggaatttgaagaagcgcatgtgacccgccagaccgtggtggcgctggcggcgcaggaaggcgaactgcatattgtgctggcgggcgcgattccggtgaccgtggcgggcaccaccctgaccctgaccagcggccatctgaaatgccgcatgaaactggataaactgaaaattaaaggcagcacctatctgatgtgcaaagataaatttgcgtttgcgaaaaacccggtggataccggccatggcaccattgtgaccgaagtgcagtatgcgggcagcgatggcccgtgccgcattccgattaccatgaccgaaaacctgcatgatctgaccccgattggccgcctggtgaccgtgaacccgtttgtgccgagcagcgaaaccgcgcagaaaattctgattgaactggaaccgccgtttggcaccagctttattctggtgggcaccggcccgaaccaggtgaaatatcagtggcataaaagcggcagc

SEQ ID NO: 72 - protein, 406 aa, Orthoflavivirus spondweni
IRCIGIGNRDFIEGMSGGTWVDIVLEHGGCVTVMSNDKPTLDFELVTTTASNMAEVRSYCYEANISEMASDSRCPTQGEAYLDKMADSQFVCKRGYVDRGWGNGCGLFGKGSIVTCAKFTCVKKLTGKSIQPENLEYRVLVSVHASQHGGMINNDTNHQHDKENRARIDITASAPRVEVELGSFGSFSMECEPRSGLNFGDLYYLTMNNKHWLVNRDWFHDLSLPWHTGATSNNHHWNNKEALVEFREAHAKKQTAVVLGSQEGAVHAALAGALEAESDGHKATIYSGHLKCRLKLDKLRLKGMSYALCTGAFTFARTPSETIHGTATVELQYAGEDGPCKVPIVITSDTNSMASTGRLITANPVVTESGANSKMMVEIDPPFGDSYIIVGTGTTKITHHWHRAGS

SEQ ID NO: 73 - protein, 406 aa, Synthetic construct
IRCICIGNRDFIEGMSGGTWVDIVLEHGGCVTVMSNDKPTLDFELVTTTASNMAEVRSYCYEANISEMASDSRCPTQGEAYLDKMADSQFVCKRGYVDRGWCNGCGLFGKGSIVTCAKFTCVKKLTGKSIQPENLEYRVLVSVHASQHGGMINNDTNHQHDKENRARIDITASAPRVEVELGSFGSFSMECEPRSGLNFGDLYYLTMNNKHWLVNRDWFHDLSLPWHTGATSNNHHWNNKEALVEFREAHAKKQTAVVLGSQEGAVHAALAGALEAESDGHKATIYSGHLKCRLKLDKLRLKGMSYALCTGAFTFARTPSETIHGTATVELQYAGEDGPCKVPIVITSDTNSMASTGRLITANPVVTESGANSKMMVEIDPPFGDSYIIVGTGTTKITHHWHRAGS

SEQ ID NO: 74 - DNA, 1218 bp, Synthetic construct
  source 1..1218: mol_type = other DNA
attcgctgcatttgcattggcaaccgcgattttattgaaggcatgagcggcggcacctgggtggatattgtgctggaacatggcggctgcgtgaccgtgatgagcaacgataaaccgaccctggattttgaactggtgaccaccaccgcgagcaacatggcggaagtgcgcagctattgctatgaagcgaacattagcgaaatggcgagcgatagccgctgcccgacccagggcgaagcgtatctggataaaatggcggatagccagtttgtgtgcaaacgcggctatgtggatcgcggctggtgcaacggctgcggcctgtttggcaaaggcagcattgtgacctgcgcgaaatttacctgcgtgaaaaaactgaccggcaaaagcattcagccggaaaacctggaatatcgcgtgctggtgagcgtgcatgcgagccagcatggcggcatgattaacaacgataccaaccatcagcatgataaagaaaaccgcgcgcgcattgatattaccgcgagcgcgccgcgcgtggaagtggaactgggcagctttggcagctttagcatggaatgcgaaccgcgcagcggcctgaactttggcgatctgtattatctgaccatgaacaacaaacattggctggtgaaccgcgattggtttcatgatctgagcctgccgtggcataccggcgcgaccagcaacaaccatcattggaacaacaaagaagcgctggtggaatttcgcgaagcgcatgcgaaaaaacagaccgcggtggtgctgggcagccaggaaggcgcggtgcatgcggcgctggcgggcgcgctggaagcggaaagcgatggccataaagcgaccatttatagcggccatctgaaatgccgcctgaaactggataaactgcgcctgaaaggcatgagctatgcgctgtgcaccggcgcgtttacctttgcgcgcaccccgagcgaaaccattcatggcaccgcgaccgtggaactgcagtatgcgggcgaagatggcccgtgcaaagtgccgattgtgattaccagcgataccaacagcatggcgagcaccggccgcctgattaccgcgaacccggtggtgaccgaaagcggcgcgaacagcaaaatgatggtggaaattgatccgccgtttggcgatagctatattattgtgggcaccggcaccaccaaaattacccatcattggcatcgcgcgggcagc

SEQ ID NO: 75 - RNA, 1218 nt, Synthetic construct
  source 1..1218: mol_type = other RNA
attcgctgcatttgcattggcaaccgcgattttattgaaggcatgagcggcggcacctgggtggatattgtgctggaacatggcggctgcgtgaccgtgatgagcaacgataaaccgaccctggattttgaactggtgaccaccaccgcgagcaacatggcggaagtgcgcagctattgctatgaagcgaacattagcgaaatggcgagcgatagccgctgcccgacccagggcgaagcgtatctggataaaatggcggatagccagtttgtgtgcaaacgcggctatgtggatcgcggctggtgcaacggctgcggcctgtttggcaaaggcagcattgtgacctgcgcgaaatttacctgcgtgaaaaaactgaccggcaaaagcattcagccggaaaacctggaatatcgcgtgctggtgagcgtgcatgcgagccagcatggcggcatgattaacaacgataccaaccatcagcatgataaagaaaaccgcgcgcgcattgatattaccgcgagcgcgccgcgcgtggaagtggaactgggcagctttggcagctttagcatggaatgcgaaccgcgcagcggcctgaactttggcgatctgtattatctgaccatgaacaacaaacattggctggtgaaccgcgattggtttcatgatctgagcctgccgtggcataccggcgcgaccagcaacaaccatcattggaacaacaaagaagcgctggtggaatttcgcgaagcgcatgcgaaaaaacagaccgcggtggtgctgggcagccaggaaggcgcggtgcatgcggcgctggcgggcgcgctggaagcggaaagcgatggccataaagcgaccatttatagcggccatctgaaatgccgcctgaaactggataaactgcgcctgaaaggcatgagctatgcgctgtgcaccggcgcgtttacctttgcgcgcaccccgagcgaaaccattcatggcaccgcgaccgtggaactgcagtatgcgggcgaagatggcccgtgcaaagtgccgattgtgattaccagcgataccaacagcatggcgagcaccggccgcctgattaccgcgaacccggtggtgaccgaaagcggcgcgaacagcaaaatgatggtggaaattgatccgccgtttggcgatagctatattattgtgggcaccggcaccaccaaaattacccatcattggcatcgcgcgggcagc

SEQ ID NO: 76 - protein, 8 aa, Synthetic construct
GGGSGGGS

(19) United States
(12) Patent Application Publication
(10) Pub. No.: US 2026/0184744 A1
(43) Pub. Date: Jul. 2, 2026

(54) ENGINEERED FLAVIVIRUS ENVELOPE GLYCOPROTEIN IMMUNOGENIC COMPOSITIONS AND METHODS OF USE

(71) Applicants: University of Maryland, Baltimore, Baltimore, MD (US); University of Maryland, College Park, College Park, MD (US)

(72) Inventors: Yuxing Li, Boyds, MD (US); Andrey Galkin, Germantown, MD (US); Alexander K. Andrianov, Gaithersburg, MD (US); Yimeng Wang, Belmont, MA (US)

(21) Appl. No.: 19/120,647

(22) PCT Filed: Oct. 10, 2023
(86) PCT No.: PCT/US2023/076441

§ 371 Date: Apr. 11, 2025

Related U.S. Application Data
(60) Provisional application No. 63/415,141, filed on Oct. 11, 2022.

(51) Int. Cl. C07K 14/18; A61K 39/00; A61K 39/12; A61P 31/14; C12N 15/11; C12N 15/86

(52) U.S. Cl. CPC C07K 14/1825; A61K 39/12; A61P 31/14; C12N 15/11; C12N 15/86; A61K 2039/55511; A61K 2039/55555; A61K 2039/575; C07K 2319/00; C12N 2770/24122; C12N 2770/24134

(57) ABSTRACT

The present invention provides polypeptides, and polypeptide dimers, comprising a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide, nucleic acids, vectors, host cells, and pharmaceutical compositions comprising the same, and methods of inducing an immune response in subjects by administering the pharmaceutical compositions of the invention.

### CROSS REFERENCE TO RELATED APPLICATIONS

This application claims the benefit of U.S. Provisional Appl. No. 63/415,141, filed on Oct. 11, 2022, the contents of which are incorporated by reference herein in their entirety.

### STATEMENT OF FEDERALLY SPONSORED RESEARCH AND DEVELOPMENT

This invention was made with government support under Grant Number AI175439 awarded by the National Institutes of Health. The government has certain rights in the invention.

### INCORPORATION-BY-REFERENCE OF MATERIAL SUBMITTED ELECTRONICALLY

Incorporated by reference in its entirety herein is a computer-readable sequence listing and identified as follows: One 124,825 Byte XML file named “Sequence_listing.xml,” created on Oct. 10, 2023.

### FIELD OF THE INVENTION

The field of the invention relates to medicine and infectious diseases, in particular vaccines to treat or prevent flavivirus infection.

### BACKGROUND OF THE INVENTION

Flaviviruses are vector-borne RNA viruses that can emerge unexpectedly in human populations and cause a spectrum of potentially severe diseases including hepatitis, vascular shock syndrome, encephalitis, acute flaccid paralysis, and congenital abnormalities and fetal death. This epidemiological pattern has occurred numerous times during the last seventy years, including epidemics of Dengue virus and West Nile virus, and the most recent explosive epidemic of Zika virus in the Americas. Flaviviruses now are globally distributed and infect up to 400 million people annually.

Zika virus (ZIKV) is a member of flavivirus family that emerged as an infectious agent causing global health crisis during recent epidemics. ZIKV infection can cause Guillain-Barr6 syndrome in adults, and severe fetal neuro-malformations and fetal death during pregnancy (Diamond et al., Annu Rev Med, (2019), 70:121-135). ZIKV infection is primarily transmitted by mosquito bite, while sexual transmission and vertical transmission from infected pregnant women to fetus also contribute to the recent epidemic. Ideally, an effective ZIKV vaccine should provide sterilizing immunity that blocks the initial viral dissemination to prevent subsequent infection-caused morbidity. Currently, there is no approved ZIKV vaccine for disease prevention.

The membrane (M) and envelope protein (E) expressed as the prM-E form is a common antigen choice for current vaccine candidates against ZIKV, as neutralizing antibodies (nAb) against prM-E can prevent viral entry. However, such nascent PrM-E based ZIKV vaccines have the potential of increasing the infectiousness of the dengue virus (DENV), another flavivirus of which endemic area largely overlaps with areas affected by ZIKV epidemic. Due to the high degree of sequence homology between the E proteins of ZIKV and DENV, the ZIKV prM-E vaccine may stimulate the production of antibodies that are weak- or non-neutralizing but cross-reactive with the DENV E protein (Priyamvada et al., Emerg Microbes Infect, (2017) 6:e33). In the event of a subsequent dengue virus infection, antibody-dependent enhancement (ADE) can occur when the suboptimal anti-ZIKV antibodies bind to the DENV virus, which thereby enhance the entry of DENV into host cells and exacerbate dengue symptoms. Strategies to prevent the induction of ADE-prone antibodies have been described recently for modified ZIKV immunogens, which unfortunately display suboptimal protection efficacy in small animals (Richner et al., Cell, (2017), 169:176; Slon-Campos et al., Nat Immunol, (2019), 20:1291-1298).

The ZIKV E protein is the focus of nAb and vaccine development. The ZIKV E protein, consisting of three ectodomains (DI, DII, and DIII), displays homo-dimeric conformation on the mature virion. There are three major nAb epitopes on E protein: (i) E-dimer epitope (EDE), a quaternary epitope formed across the interface of two E protomers Diamond et al., Annu Rev Med, (2019). 70:121-135; Priyamvada el al., Emerg Microbes Infect, (2017) 6:e33); (ii) domain III lateral ridge epitope, DIII LR (Richner et al., Cell, (2017), 169:176; Slon-Campos et al., Nat Immunol, (2019), 20:1291-1298); and (iii) a quaternary epitope located on DII at the dimer-dimer interface represented by mAb ZIKV-117 (Hasan S S et al., Nat Commun, (2017), 8:14722; Sapparapu G et al., Nature, (2016), 540:443-447).

An immunodominant and suboptimal epitope, namely fusion loop epitope (FLE), consisting of 11 amino acid residues within DII and sharing high degree of sequence homology with other flavivirus E proteins, elicits highly abundant antibodies that show broad flavivirus cross-reactivity but low or absent neutralization capacity (Diamond et al., Annu Rev Med, (2019), 70:121-135). ZIKV FLE-directed antibody responses in immune sera from mice immunized with nascent ZIKV E-based vaccine candidates were shown to be the major cross-reactive antibody subset mediating the ADE effect on DENV infection (Richner et al., Cell, (2017), 169:176; Slon-Campos et al., Nat Immunol, (2019), 20:1291-1298; Dai L et al., Nat Immunol, (2021), 22:958-968).

What is needed are new flavivirus vaccines, including new ZIKV vaccines, that elicit potent and neutralizing antibody responses but without the adverse effects of increasing the infectiousness of other flaviviruses, including dengue virus, and without the production of antibodies cross-reactive with other flavivirus E proteins, including the DENV E protein.

The foregoing description of the background is provided to aid in understanding the invention, and is not admitted to be or to describe prior art to the invention.

### SUMMARY OF THE INVENTION

It is to be understood that both the foregoing general description of the invention and the following detailed description are exemplary, and thus do not restrict the scope of the invention.

In some embodiments, the present invention provides novel flavivirus pharmaceutical compositions, such as vaccines, and methods with improved immunogenicity and reduced antibody-dependent enhancement (ADE) potential for other flavivirus infections, such as dengue infection. In some embodiments, the invention provides engineered flavivirus envelope (E) proteins formulated in optimized adjuvant, and which provide protective immunity and abolished ADE potential.

In some embodiments, the flavivirus is selected from the group consisting of Zika virus, dengue virus, yellow fever virus, Japanese encephalitis virus, St. Louis encephalitis virus, tick-borne encephalitis virus, West Nile virus, Ilheus virus, Powassan virus, Wesselsbron virus, Usutu virus, Rocio virus, and Spondweni virus.

In one aspect, the invention provides a polypeptide or a polypeptide dimer comprising a modified flavivirus virus envelope E polypeptide, wherein the modified flavivirus virus envelope E polypeptide comprises i) a cysteine residue in a fusion loop epitope of ectodomain DII; and ii) a cysteine residue in ectodomain DI located in an N-terminal portion of the polypeptide.

In some embodiments, the polypeptide is present in a dimer consisting of two modified flavivirus virus envelope E polypeptides that have the same amino acid sequence.

In some embodiments, the cysteine residue in the fusion loop epitope of ectodomain DII on one polypeptide chain of the dimer forms an interchain disulfide bond with the cysteine residue in the ectodomain DI on the other polypeptide chain of the dimer.

In some embodiments, the modified flavivirus envelope E polypeptide comprises ectodomains DI. DII, and DIII, and lacks stem and anchor domains.

In some embodiments, the flavivirus is a Zika virus and the cysteine residues are present at amino acid position numbers 5 and 102 with reference to SEQ ID NO:1.

In some embodiments, the modified Zika virus envelope E polypeptide comprises an amino acid sequence at least 90% identical to SEQ ID NO:2.

In some embodiments, the modified Zika virus envelope E polypeptide comprises an amino acid sequence of SEQ ID NO:2, or an antigenic fragment or a variant thereof.

In some embodiments, the flavivirus is a West Nile virus, and the modified West Nile virus envelope E polypeptide comprises cysteine residues present at amino acid position numbers 5 and 102 with reference to SEQ ID NO:7.

In some embodiments, the flavivirus is a West Nile virus, wherein the modified West Nile virus envelope E polypeptide comprises an amino acid sequence of SEQ ID NO:8, or an antigenic fragment or a variant thereof.

In some embodiments, the flavivirus is a West Nile virus and the modified West Nile virus envelope E polypeptide comprises an amino acid sequence at least 90% identical to SEQ ID NO:8.

In some embodiments, the flavivirus is a dengue virus serotype 1, and the modified dengue virus serotype 1 envelope E polypeptide comprises cysteine residues present at amino acid position numbers 5 and 102 with reference to SEQ ID NO:28.

In some embodiments, the modified dengue virus serotype 3 polypeptide comprises an amino acid sequence of SEQ ID NO:29, or an antigenic fragment or a variant thereof.

In some embodiments, the modified dengue virus serotype 1 envelope E polypeptide comprises an amino acid sequence at least 90% identical to SEQ ID NO:29.

In some embodiments, the flavivirus is a dengue virus serotype 2, and the modified dengue virus serotype 2 envelope E polypeptide comprises cysteine residues present at amino acid position numbers 5 and 102 with reference to SEQ ID NO:24.

In some embodiments, the modified dengue virus serotype 2 polypeptide comprises an amino acid sequence of SEQ ID NO:25, or an antigenic fragment or a variant thereof.

In some embodiments, the modified dengue virus serotype 2 envelope E polypeptide comprises an amino acid sequence at least 90% identical to SEQ ID NO:25.

In some embodiments, the flavivirus is a dengue virus serotype 3, and the modified dengue virus serotype 3 envelope E polypeptide comprises cysteine residues present at amino acid position numbers 5 and 102 with reference to SEQ ID NO:20.

In some embodiments, the modified dengue virus serotype 3 polypeptide comprises an amino acid sequence of SEQ ID NO:21, or an antigenic fragment or a variant thereof.

In some embodiments, the modified dengue virus serotype 3 envelope E polypeptide comprises an amino acid sequence at least 90% identical to SEQ ID NO:21.

In some embodiments, the flavivirus is a dengue virus serotype 4, and the modified dengue virus serotype 4 envelope E polypeptide comprises cysteine residues present at amino acid position numbers 5 and 102 with reference to SEQ ID NO:32.

In some embodiments, the modified dengue virus serotype 4 polypeptide comprises an amino acid sequence of SEQ ID NO:33, or an antigenic fragment or a variant thereof.

In some embodiments, the modified dengue virus serotype 4 envelope E polypeptide comprises an amino acid sequence at least 90% identical to SEQ ID NO:33.

In some embodiments, the flavivirus is a yellow fever virus, and the modified yellow fever virus envelope E polypeptide comprises cysteine residues present at amino acid position numbers 5 and 102 with reference to SEQ ID NO:36.

In some embodiments, the modified yellow fever virus envelope E polypeptide comprises an amino acid sequence of SEQ ID NO:37, or an antigenic fragment or a variant thereof.

In some embodiments, the modified yellow fever virus envelope E polypeptide comprises an amino acid sequence at least 90% identical to SEQ ID NO:37.

In some embodiments, the flavivirus is a Japanese encephalitis virus, and the modified Japanese encephalitis virus envelope E polypeptide comprises cysteine residues present at amino acid position numbers 5 and 102 with reference to SEQ ID NO:40.

In some embodiments, the modified Japanese encephalitis virus envelope E polypeptide comprises an amino acid sequence of SEQ ID NO:41, or an antigenic fragment or a variant thereof.

In some embodiments, the modified Japanese encephalitis virus envelope E polypeptide comprises an amino acid sequence at least 90% identical to SEQ ID NO:41.

In some embodiments, the flavivirus is a St. Louis encephalitis virus, and the modified St. Louis encephalitis virus envelope E polypeptide comprises cysteine residues present at amino acid position numbers 5 and 102 with reference to SEQ ID NO:44.

In some embodiments, the modified St. Louis encephalitis virus envelope E polypeptide comprises an amino acid sequence of SEQ ID NO:45, or an antigenic fragment or a variant thereof.

In some embodiments, the modified St. Louis encephalitis virus envelope E polypeptide comprises an amino acid sequence at least 90% identical to SEQ ID NO:45.

In some embodiments, the flavivirus is a tick-borne encephalitis virus, and the modified tick-borne encephalitis virus envelope E polypeptide comprises cysteine residues present at amino acid position numbers 5 and 102 with reference to SEQ ID NO:48.

In some embodiments, the modified tick-borne encephalitis virus envelope E polypeptide comprises an amino acid sequence of SEQ ID NO:49, or an antigenic fragment or a variant thereof.

In some embodiments, the modified tick-borne encephalitis virus envelope E polypeptide comprises an amino acid sequence at least 90% identical to SEQ ID NO:49.

In some embodiments, the flavivirus is an Ilheus virus, and the modified Ilheus virus envelope E polypeptide comprises cysteine residues present at amino acid position numbers 5 and 102 with reference to SEQ ID NO:52.

In some embodiments, the modified Ilheus virus envelope E polypeptide comprises an amino acid sequence of SEQ ID NO:53, or an antigenic fragment or a variant thereof.

In some embodiments, the modified Ilheus virus envelope E polypeptide comprises an amino acid sequence at least 90% identical to SEQ ID NO:53.

In some embodiments, the flavivirus is a Wesselsbron virus, and the modified Wesselsbron virus envelope E polypeptide comprises cysteine residues present at amino acid position numbers 5 and 102 with reference to SEQ ID NO:56.

In some embodiments, the modified Wesselsbron virus envelope E polypeptide comprises an amino acid sequence of SEQ ID NO:57, or an antigenic fragment or a variant thereof.

In some embodiments, the modified Wesselsbron virus envelope E polypeptide comprises an amino acid sequence at least 90% identical to SEQ ID NO:57.

In some embodiments, the flavivirus is an Usutu virus, and the modified Usutu virus envelope E polypeptide comprises cysteine residues present at amino acid position numbers 5 and 102 with reference to SEQ ID NO:60.

In some embodiments, the modified Usutu virus envelope E polypeptide comprises an amino acid sequence of SEQ ID NO:61, or an antigenic fragment or a variant thereof.

In some embodiments, the modified Usutu virus envelope E polypeptide comprises an amino acid sequence at least 90% identical to SEQ ID NO:61.

In some embodiments, the flavivirus is a Powassan virus, and the modified Powassan virus envelope E polypeptide comprises cysteine residues present at amino acid position numbers 5 and 102 with reference to SEQ ID NO:64.

In some embodiments, the modified Powassan virus envelope E polypeptide comprises an amino acid sequence of SEQ ID NO:65, or an antigenic fragment or a variant thereof.

In some embodiments, the modified Powassan virus envelope E polypeptide comprises an amino acid sequence at least 90% identical to SEQ ID NO:65.

In some embodiments, the flavivirus is a Rocio virus, and the modified Rocio virus envelope E polypeptide comprises cysteine residues present at amino acid position numbers 5 and 102 with reference to SEQ ID NO:68.

In some embodiments, the modified Rocio virus envelope E polypeptide comprises an amino acid sequence of SEQ ID NO:69, or an antigenic fragment or a variant thereof.

In some embodiments, the modified Rocio virus envelope E polypeptide comprises an amino acid sequence at least 90% identical to SEQ ID NO:69.

In some embodiments, the flavivirus is a Spondweni virus, and the modified Spondweni virus envelope E polypeptide comprises cysteine residues present at amino acid position numbers 5 and 102 with reference to SEQ ID NO:72.

In some embodiments, the modified Spondweni virus envelope E polypeptide comprises an amino acid sequence of SEQ ID NO:73, or an antigenic fragment or a variant thereof.

In some embodiments, the modified Spondweni virus envelope E polypeptide comprises an amino acid sequence at least 90% identical to SEQ ID NO:73.

In another aspect, the invention provides a pharmaceutical composition comprising an effective amount of a polypeptide dimer comprising a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide.

In some embodiments, the pharmaceutical composition further comprises an effective amount of an adjuvant.

In some embodiments, the adjuvant is a polyphosphazene adjuvant. In some embodiments, the adjuvant is selected from PCPP, PCEP and combinations thereof.

In some embodiments, the composition further comprises a TLR7/8 agonist.

In some embodiments, the polypeptide dimer, adjuvant and TLR7/8 agonist are in a complex.

In some embodiments, the TLR7/8 agonist is R-848.

In another aspect, the invention provides a nucleic acid encoding a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide.

In some embodiments, the nucleic acid is mRNA.

In another aspect, the invention provides a vector encoding the modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide. In some embodiments, the vector is a viral vector. In some embodiments, the vector is a eukaryotic expression vector.

In another aspect, the invention provides a host cell comprising a nucleic acid encoding a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide.

In another aspect, the invention provides a pharmaceutical composition comprising an effective amount of a nucleic acid or an effective amount of a vector encoding a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide.

In some embodiments, the nucleic acid is formulated as a nanoparticle. In some embodiments, the nanoparticle is a solid lipid nanoparticle.

In another aspect, the invention provides a method of inducing an immune response in a subject, comprising administering to the subject a pharmaceutical composition comprising an effective amount of a polypeptide dimer comprising a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide.

In another aspect, the invention provides a method of inducing an immune response in a subject, comprising administering to the subject a pharmaceutical composition comprising an effective amount of a nucleic acid or an effective amount of a vector encoding a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide.

In some embodiments, the subject is a human.

In some embodiments, the pharmaceutical composition is administered to the subject more than one time. In some embodiments, the subject is administered a first priming dose of the pharmaceutical composition, and is subsequently boosted with a second dose of the pharmaceutical composition after the first priming dose. In some embodiments, the second dose is administered from 2-8 weeks after the first dose.

In some embodiments, the composition is administered via a parenteral route. In some embodiments, the parenteral route is selected from the group consisting of subcutaneous (s.c), intradermal (i.d.), intramuscular (i.m.), intraperitoneal (i.p.), and intravenous (i.v.) injection.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. ZIKV E-based immunogen design. Top, Schematic of the ZIKV E protein within the context of the viral polyprotein. The localization of the residues that form interchain disulfide bonds on mutation to cysteine is noted. D, domain; C, capsid protein; prM, precursor membrane protein; NS, non-structural protein; sRecE, recombinant soluble form of ecto portion of ZIKV E protein (residues 1-405); WT, nascent sRecE; CC_FLE, engineered inter-protomer disulfide bond tethering the fusion loop epitope (FLE) to the N-terminus of sRecE conferred by mutations G5C & G102C; CC_Core, engineered inter-protomer disulfide bond connecting the central part of sRecE conferred by mutation A264C; DI-DIII, sRecE residues 51-133 and 195-284 are deleted and replaced with a (G₄S)₁ (GGGS) and a (G₄S)₂ (GGGSGGGS) linker, respectively; DIII, consists of residues 303-405; DIII-NP, residues 303-406 and ferritin motif connected by peptide linker.

FIG. 2. Biochemical and antigenicity characterization of ZIKV sRecE mutants CC_FLE & CC_Core. (A) Size exclusion chromatography profiles; (B) ELISA binding to reference ZIKV mAbs, including EDE epitope mAbs (EDE1-C8, SMZAb2), DIII LR (ZV67, Z004), and FLE (2A10G6, E60); (C) ELISA binding EC₅₀ value heat map.

FIG. 3. Characterization of ZIKV sRecE_DI-DIII, DIII, & DIII-NP proteins. (A) Bio-Layer Interferometry (BLI) curves of sRecE_DI-DIII & WT. Antibodies were initially captured on anti-human Fc BLI probe, followed by immersing into wells containing sRecE ranging from 62.5-250 nM in concentration. BLI curves generated with a panel of DIII-specific mAb (Z004) or (B) FLE-specific mAb, 4G2. (C). Binding kinetic parameters for sRecE_DI-DIII & WT to DIII-specific mAbs (Z004, Z006) or FLE-specific mAb, 4G2. (D) BLI analysis of sRecE_DIII-Nano & DIII. BLI curves generated with DIII-specific mAb ZV67. (E) Binding kinetic parameters derived from (D).

FIG. 4. Immunogen screening in C57BL/6 mice. (A) Immunization schedule. C57BL/6 mice (N=4/group) were immunized with 20 μg of immunogen formulated in adjuplex as adjuvant via subcutaneous (s.c.) route on days 0 and 28. Sera on day 42 were tested for neutralization capacity. DIII-NP/DIII group mice were primed with DIII-NP on day 0, and boosted with DIII on day 28. (B) Day 42 sera neutralization ID₅₀ titers against pseudotype ZIKV H/PF/2013. Shown is the ID₅₀ titer of individual serum sample, with geometric mean±SD.

FIG. 5. Adjuvant optimization (A) In-house polyphosphazene-based adjuvant formulation developed by Dr. Andrianov at IBBR/UMCP. Shown is PCEP polymer complex with TLR7/8 agonist R848 and antigen (Ag) for inoculation. (B) Immunization schedule. 20 g of immunogen formulated in various adjuvants were inoculated via s.c. route. Sera on Day 35 were collected and used for virus neutralization assay, while sera on Day 42 were used for passively transfer study. (C) Day 35 sera ID₅₀ titers against pseudotype ZIKV H/PF/2013. (D) Survival curve of ZIKV challenged AG129 mice receiving day 42 serum transferred from immune mice in (B). AG129 mice (n=6/group) were infused with 200 μl of immune sera of day 42 in (B) via intraperitoneal (i.p.) route from selected groups, challenged with ZIKV FSS13025 (10⁴ PFU) via s.c. route 1 hour post serum infusion. Mantel-Cox log-rank test with *p<0.05 indicating statistical significance of survival between donor mice from PBS and CC_FLE formulated with PCEP+R848.

FIG. 6. CC_FLE immunized mouse sera showed minimal or abolished ADE effect on DENV infection. (A). Scheme of ADE effect assay. Selected Day 42 immune sera (FIG. 5B) were co-incubated with (B) DENV-1 or (C) DENV-2 pseudotype virus and K562 cells (FcγRIIA+), followed by virus entry signal evaluation. WT vs. PBS group sera comparison, significant difference by one-way ANOVA Kruskal-Wallis test (** p<0.01, ***p<0.001).

FIG. 7. In vivo efficacy study of CC_FLE formulated in PCEP+R848 adjuvant in BALB/c mice. (A) Study design scheme. 20 μg of immunogen formulated in PCEP+R848 were inoculated via s.c. route. ZIKV Puerto Rico 2015 (2×10⁶ TCID₅₀) was used for challenge via s.c. route. (B) Day 35 sera ID₅₀ titers against pseudotype ZIKV H/PF/2013. Geometric mean of ID₅₀±SD for each group is denoted. Significant difference by one-way ANOVA Kruskal-Wallis test (** p<0.01, ***p<0.001).

FIG. 8. CC_FLE formulated in PCEP+R848 adjuvant conferred nearly sterilizing protection in BALB/c mice. (A) 1 DPI immune mouse serum virus load (VL) assayed as ZIKV RNA copy number/ml. (B) Lymph node VL on 10 DPI. Geometric mean of VL±SD for each group is denoted. Significant difference by one-way ANOVA Kruskal-Wallis test (*p<0.05, ** p<0.01, ***p<0.001). (C) Inverse correlation of serum nAb titers and serum virus load.

FIG. 9. CC_FLE linkage is applied to other flavivirus sRecEs. (A) Amino acid sequence alignment of the sRecE N terminus (N-term) and FLE of selected mosquito-borne flaviviruses. Dots indicate residues identical to the corresponding ZIKV sRecE residues. G5 and G102, mutated to C (cysteine) in the CC_FLE design to form disulfide linkage are highlighted in red font; DENV, Dengue Virus; WNV, West Nile Virus; JEV, Japanese Encephalitis Virus; YFV, Yellow Fever Virus; (B) The formation of CC_FLE linkage of WNV CC_FLE sE indicated by size exclusion chromatography dimeric profile; (C) WNV CC_FLE sRecE displays attenuated binding to FLE-specific mAbs, shown is the ELISA binding EC₅₀ value heat map of WNV sRecE WT and CC_FLE mutant.

### DETAILED DESCRIPTION OF THE INVENTION

Reference will now be made in detail to the presently preferred embodiments of the invention which, together with the drawings and the following examples, serve to explain the principles of the invention. These embodiments are described in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Identical” or “identity,” as used herein in the context of two or more polypeptide or polynucleotide sequences, can mean that the amino acid or nucleotide sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

“Sample,” “test sample,” “specimen,” “sample from a subject,” and “patient sample” as used herein may be used interchangeable and may be a sample of blood, such as whole blood, tissue, urine, serum, plasma, amniotic fluid. cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Variant” is used herein to describe a peptide or polypeptide that differs in amino acid sequence by the insertion, deletion, or conservative substitution of amino acids, but retain at least one biological activity. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. “Vector” is used herein to describe a nucleic acid molecule that can transport another nucleic acid to which it has been linked. One type of vector is a “plasmid”, which refers to a circular double-stranded DNA loop into which additional DNA segments may be ligated.

Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find in the context of the present disclosure.

The term “attenuated virus” as used herein, refers to a virus with compromised virulence in the intended recipient (e.g., human or animal recipient). More specifically, an attenuated virus has a decreased or weakened ability to produce disease while retaining the ability to stimulate an immune response similar to the wild-type (non-attenuated) virus.

### Polypeptides

In one embodiment, the invention provides a polypeptide or a polypeptide dimer comprising a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide.

Flaviviruses are vector-borne RNA viruses that can cause a spectrum of potentially severe diseases including hepatitis, vascular shock syndrome, encephalitis, acute flaccid paralysis, and congenital abnormalities and fetal death. The flavivirus is not limiting. In some embodiments, the flavivirus is selected from the group consisting of Zika virus, dengue virus, yellow fever virus, Japanese encephalitis virus, St. Louis encephalitis virus, tick-borne encephalitis virus, West Nile virus, Ilheus virus, Powassan virus, Wesselsbron virus, Usutu virus, Rocio virus, and Spondweni virus.

The parental or wild-type flavivirus envelope E polypeptide that can be modified is not limiting. Any envelope E polypeptide derived from any flavivirus or strain can be used in the methods and compositions described herein. In some embodiments, the flavivirus is an isolate from an infected subject during an outbreak, such as a Zika virus outbreak.

Zika virus is a flavivirus closely related to Dengue virus and is similarly transmitted by the Aedes species mosquito, although other arthropod vectors for Zika virus are possible. Since it was first isolated from a Rhesus monkey in the Zika forest of Uganda in 1947, there were very few reported incidents of human infection, especially outside of the endemic regions of Africa and Asia until a large outbreak in French Polynesia in 2007 (Haddow et al. PLoS Neglected Tropical Diseases (2012) 6(2), Malone et al. PLoS Neglected Tropical Diseases (2016) 10(3),). The virus has since spread through islands of the Pacific, including Oceania, and into South and Central America (WHO “Zika Situation Report” Feb. 5, 2016).

In some embodiments, the flavivirus is a Zika virus strain isolated from Africa or from the African virus lineage. In some embodiments, the flavivirus is a Zika virus strain isolated from Asia or from the Asian lineage (includes also strains from French Polynesia). In some embodiments, the flavivirus is a Zika virus strain isolated from the Americas (South America, Central America, or North America), such as a Suriname Zika virus strain. In some embodiments, the Zika virus has an RNA genome corresponding (but not limited) to the DNA sequence provided by GenBank Accession No. AY632535.2, KU321639.1, KU497555.1, KU501215.1, KU509998.1, KU527068.1, KU681081.3, KU681082.3, KU707826.1, KU744693.1, or LC002520.1. In some embodiments, the Zika virus parental envelope E polypeptide sequence (lacking the stem and anchor domains) comprises an amino acid sequence of SEQ ID NO:1 and is encoded by DNA and RNA sequences shown in SEQ ID NOS:3 and 5, respectively.

In some embodiments, the flavivirus is a Zika virus and the modified Zika virus envelope E polypeptide comprises cysteine residues present at amino acid position numbers 5 and 102 with reference to SEQ ID NO: 1. In some embodiments, the modified Zika virus envelope E polypeptide comprises an amino acid sequence of SEQ ID NO:2, or an antigenic fragment or a variant thereof. In some embodiments, the modified Zika virus envelope E polypeptide comprises an amino acid sequence at least 90% identical to SEQ ID NO:2. In some embodiments, the modified Zika virus envelope E polypeptide is encoded by a DNA sequence comprising SEQ ID NO:4. In some embodiments, the modified Zika virus envelope E polypeptide is encoded by an RNA sequence comprising SEQ ID NO:6.

In some embodiments, the flavivirus is a West Nile virus. In some embodiments, the West Nile virus parental envelope E polypeptide sequence (lacking the stem and anchor domains) comprises an amino acid sequence of SEQ ID NO:7 (see GenBank Accession No. AAM70028). In some embodiments, the modified West Nile virus envelope E polypeptide comprises cysteine residues at amino acid position numbers 5 and 102 with reference to SEQ ID NO:7. In some embodiments, the modified West Nile virus envelope E polypeptide comprises an amino acid sequence of SEQ ID NO:8, or an antigenic fragment or a variant thereof. In some embodiments, the modified West Nile virus envelope E polypeptide comprises an amino acid sequence at least 90% identical to SEQ ID NO:8. In some embodiments, the modified West Nile virus envelope E polypeptide is encoded by a DNA sequence comprising SEQ ID NO:9. In some embodiments, the modified West Nile virus envelope E polypeptide is encoded by an RNA sequence comprising SEQ ID NO: 10.

In some embodiments, the flavivirus is a dengue virus. Various serotypes of dengue virus are known, and include, for example, serotypes 1-4.

In some embodiments, the dengue virus has serotype 1. In some embodiments, the dengue virus serotype 1 parental envelope E polypeptide sequence (lacking the stem and anchor domains) comprises an amino acid sequence of SEQ ID NO:28 (see GenBank Accession No.: BCG29749). In some embodiments, the modified dengue virus serotype 1 envelope E polypeptide comprises cysteine residues present at amino acid position numbers 5 and 102 with reference to SEQ ID NO:28. In some embodiments, the modified dengue virus serotype 1 polypeptide comprises an amino acid sequence of SEQ ID NO:29, or an antigenic fragment or a variant thereof. In some embodiments, the modified dengue virus serotype 1 envelope E polypeptide comprises an amino acid sequence at least 90% identical to SEQ ID NO:29. In some embodiments, the modified dengue virus serotype 1 envelope E polypeptide is encoded by a DNA sequence comprising SEQ ID NO:30. In some embodiments, the modified dengue virus serotype 1 envelope E polypeptide is encoded by an RNA sequence comprising SEQ ID NO:31.

In some embodiments, the dengue virus has serotype 2. In some embodiments, the dengue virus serotype 2 parental envelope E polypeptide sequence (lacking the stem and anchor domains) comprises an amino acid sequence of SEQ ID NO:24 (see GenBank Accession No.: BCG29765). In some embodiments, the modified dengue virus serotype 2 envelope E polypeptide comprises cysteine residues present at amino acid position numbers 5 and 102 with reference to SEQ ID NO:24. In some embodiments, the modified dengue virus serotype 2 polypeptide comprises an amino acid sequence of SEQ ID NO:25, or an antigenic fragment or a variant thereof. In some embodiments, the modified dengue virus serotype 2 envelope E polypeptide comprises an amino acid sequence at least 90% identical to SEQ ID NO:25. In some embodiments, the modified dengue virus serotype 2 envelope E polypeptide is encoded by a DNA sequence comprising SEQ ID NO:26. In some embodiments, the modified dengue virus serotype 2 envelope E polypeptide is encoded by an RNA sequence comprising SEQ ID NO:27.

In some embodiments, the dengue virus has serotype 3. In some embodiments, the dengue virus serotype 3 parental envelope E polypeptide sequence (lacking the stem and anchor domains) comprises an amino acid sequence of SEQ ID NO:20 (see GenBank Accession No.: ABY82135). In some embodiments, the modified dengue virus serotype 3 envelope E polypeptide comprises cysteine residues present at amino acid position numbers 5 and 102 with reference to SEQ ID NO:20. In some embodiments, the modified dengue virus serotype 3 polypeptide comprises an amino acid sequence of SEQ ID NO:21, or an antigenic fragment or a variant thereof. In some embodiments, the flavivirus is a dengue virus serotype 3 and the modified dengue virus serotype 3 envelope E polypeptide comprises an amino acid sequence at least 90% identical to SEQ ID NO:21. In some embodiments, the modified dengue virus serotype 3 envelope E polypeptide is encoded by a DNA sequence comprising SEQ ID NO:22. In some embodiments, the modified dengue virus serotype 3 envelope E polypeptide is encoded by an RNA sequence comprising SEQ ID NO:23.

In some embodiments, the dengue virus has serotype 4. In some embodiments, the dengue virus serotype 4 parental envelope E polypeptide sequence (lacking the stem and anchor domains) comprises an amino acid sequence of SEQ ID NO:32 (see GenBank Accession No.: BAC77239). In some embodiments, the modified dengue virus serotype 4 envelope E polypeptide comprises cysteine residues present at amino acid position numbers 5 and 102 with reference to SEQ ID NO:32. In some embodiments, the modified dengue virus serotype 4 polypeptide comprises an amino acid sequence of SEQ ID NO:33, or an antigenic fragment or a variant thereof. In some embodiments, the modified dengue virus serotype 4 envelope E polypeptide comprises an amino acid sequence at least 90% identical to SEQ ID NO:33. In some embodiments, the modified dengue virus serotype 4 envelope E polypeptide is encoded by a DNA sequence comprising SEQ ID NO:34. In some embodiments, the modified dengue virus serotype 4 envelope E polypeptide is encoded by an RNA sequence comprising SEQ ID NO:35.

In some embodiments, the flavivirus is a yellow fever virus. In some embodiments, the yellow fever virus parental envelope E polypeptide sequence (lacking the stem and anchor domains) comprises an amino acid sequence of SEQ ID NO:36 (see GenBank Accession No.: AAA92706). In some embodiments, the modified yellow fever virus envelope E polypeptide comprises cysteine residues present at amino acid position numbers 5 and 102 with reference to SEQ ID NO:36. In some embodiments, the flavivirus is a yellow fever virus, wherein the modified yellow fever virus envelope E polypeptide comprises an amino acid sequence of SEQ ID NO:37, or an antigenic fragment or a variant thereof. In some embodiments, the modified yellow fever virus envelope E polypeptide comprises an amino acid sequence at least 90% identical to SEQ ID NO:37. In some embodiments, the modified yellow fever virus envelope E polypeptide is encoded by a DNA sequence comprising SEQ ID NO:38. In some embodiments, the modified yellow fever virus envelope E polypeptide is encoded by an RNA sequence comprising SEQ ID NO:39.

In some embodiments, the flavivirus is a Japanese encephalitis virus. In some embodiments, the Japanese encephalitis virus parental envelope E polypeptide sequence (lacking the stem and anchor domains) comprises an amino acid sequence of SEQ ID NO:40 (see GenBank Accession No.: ABQ41425). In some embodiments, the modified Japanese encephalitis virus envelope E polypeptide comprises cysteine residues present at amino acid position numbers 5 and 102 with reference to SEQ ID NO:40. In some embodiments, the modified Japanese encephalitis virus envelope E polypeptide comprises an amino acid sequence of SEQ ID NO:41, or an antigenic fragment or a variant thereof. In some embodiments, the modified Japanese encephalitis virus envelope E polypeptide comprises an amino acid sequence at least 90% identical to SEQ ID NO:41. In some embodiments, the modified Japanese encephalitis virus envelope E polypeptide is encoded by a DNA sequence comprising SEQ ID NO:42. In some embodiments, the modified Japanese encephalitis virus envelope E polypeptide is encoded by an RNA sequence comprising SEQ ID NO:43.

In some embodiments, the flavivirus is a St. Louis encephalitis virus. In some embodiments, the St. Louis encephalitis virus parental envelope E polypeptide sequence (lacking the stem and anchor domains) comprises an amino acid sequence of SEQ ID NO:44 (see GenBank Accession No.: YP_009329949). In some embodiments, the modified St. Louis encephalitis virus envelope E polypeptide comprises cysteine residues present at amino acid position numbers 5 and 102 with reference to SEQ ID NO:44. In some embodiments, the modified St. Louis encephalitis virus envelope E polypeptide comprises an amino acid sequence of SEQ ID NO:45, or an antigenic fragment or a variant thereof.

In some embodiments, the modified St. Louis encephalitis virus envelope E polypeptide comprises an amino acid sequence at least 90% identical to SEQ ID NO:45. In some embodiments, the modified St. Louis encephalitis virus envelope E polypeptide is encoded by a DNA sequence comprising SEQ ID NO:46. In some embodiments, the modified St. Louis encephalitis virus envelope E polypeptide is encoded by an RNA sequence comprising SEQ ID NO:47.

In some embodiments, the flavivirus is a tick-borne encephalitis virus. In some embodiments, the tick-borne encephalitis virus parental envelope E polypeptide sequence (lacking the stem and anchor domains) comprises an amino acid sequence of SEQ ID NO:48 (see GenBank Accession No.: CAA54069). In some embodiments, the modified tick-borne encephalitis virus envelope E polypeptide comprises cysteine residues present at amino acid position numbers 5 and 102 with reference to SEQ ID NO:48. In some embodiments, the modified tick-borne encephalitis virus envelope E polypeptide comprises an amino acid sequence of SEQ ID NO:49, or an antigenic fragment or a variant thereof. In some embodiments, the modified tick-borne encephalitis virus envelope E polypeptide comprises an amino acid sequence at least 90% identical to SEQ ID NO:49. In some embodiments, the modified tick-borne encephalitis virus envelope E polypeptide is encoded by a DNA sequence comprising SEQ ID NO:50. In some embodiments, the modified tick-borne encephalitis virus envelope E polypeptide is encoded by an RNA sequence comprising SEQ ID NO:51.

In some embodiments, the flavivirus is an Ilheus virus. In some embodiments, the Ilheus virus parental envelope E polypeptide sequence (lacking the stem and anchor domains) comprises an amino acid sequence of SEQ ID NO:52 (see GenBank Accession No.: ABQ88006). In some embodiments, the modified Ilheus virus envelope E polypeptide comprises cysteine residues present at amino acid position numbers 5 and 102 with reference to SEQ ID NO:52. In some embodiments, the modified Ilheus virus envelope E polypeptide comprises an amino acid sequence of SEQ ID NO:53, or an antigenic fragment or a variant thereof. In some embodiments, the modified Ilheus virus envelope E polypeptide comprises an amino acid sequence at least 90% identical to SEQ ID NO:53. In some embodiments, the modified Ilheus virus envelope E polypeptide is encoded by a DNA sequence comprising SEQ ID NO:54. In some embodiments, the modified Ilheus virus envelope E polypeptide is encoded by an RNA sequence comprising SEQ ID NO:55.

In some embodiments, the flavivirus is a Wesselsbron virus. In some embodiments, the Wesselsbron virus parental envelope E polypeptide sequence (lacking the stem and anchor domains) comprises an amino acid sequence of SEQ ID NO:56 (see GenBank Accession No.: UUB88388). In some embodiments, the modified Wesselsbron virus envelope E polypeptide comprises cysteine residues present at amino acid position numbers 5 and 102 with reference to SEQ ID NO:56. In some embodiments, the modified Wesselsbron virus envelope E polypeptide comprises an amino acid sequence of SEQ ID NO:57, or an antigenic fragment or a variant thereof. In some embodiments, the modified Wesselsbron virus envelope E polypeptide comprises an amino acid sequence at least 90% identical to SEQ ID NO:57. In some embodiments, the modified Wesselsbron virus envelope E polypeptide is encoded by a DNA sequence comprising SEQ ID NO:58. In some embodiments, the modified Wesselsbron virus envelope E polypeptide is encoded by an RNA sequence comprising SEQ ID NO:59.

In some embodiments, the flavivirus is an Usutu virus. In some embodiments, the Usutu virus parental envelope E polypeptide sequence (lacking the stem and anchor domains) comprises an amino acid sequence of SEQ ID NO:60 (see GenBank Accession No.: YP_164819). In some embodiments, the modified Usutu virus envelope E polypeptide comprises cysteine residues present at amino acid position numbers 5 and 102 with reference to SEQ ID NO:60. In some embodiments, the modified Usutu virus envelope E polypeptide comprises an amino acid sequence of SEQ ID NO:61, or an antigenic fragment or a variant thereof. In some embodiments, the modified Usutu virus envelope E polypeptide comprises an amino acid sequence at least 90% identical to SEQ ID NO:61. In some embodiments, the modified Usutu virus envelope E polypeptide is encoded by a DNA sequence comprising SEQ ID NO:62. In some embodiments, the modified Usutu virus envelope E polypeptide is encoded by an RNA sequence comprising SEQ ID NO:63.

In some embodiments, the flavivirus is a Powassan virus. In some embodiments, the Powassan virus parental envelope E polypeptide sequence (lacking the stem and anchor domains) comprises an amino acid sequence of SEQ ID NO:64 (see GenBank Accession No.: NP_775516). In some embodiments, the modified Powassan virus envelope E polypeptide comprises cysteine residues present at amino acid position numbers 5 and 102 with reference to SEQ ID NO:64. In some embodiments, the modified Powassan virus envelope E polypeptide comprises an amino acid sequence of SEQ ID NO:65, or an antigenic fragment or a variant thereof. In some embodiments, the modified Powassan virus envelope E polypeptide comprises an amino acid sequence at least 90% identical to SEQ ID NO:65. In some embodiments, the modified Powassan virus envelope E polypeptide is encoded by a DNA sequence comprising SEQ ID NO:66. In some embodiments, the modified Powassan virus envelope E polypeptide is encoded by an RNA sequence comprising SEQ ID NO:67.

In some embodiments, the flavivirus is a Rocio virus. In some embodiments, the Rocio virus parental envelope E polypeptide sequence (lacking the stem and anchor domains) comprises an amino acid sequence of SEQ ID NO:68 (see GenBank Accession No.: Q32ZD4). In some embodiments, the modified Rocio virus envelope E polypeptide comprises cysteine residues present at amino acid position numbers 5 and 102 with reference to SEQ ID NO:68. In some embodiments, the modified Rocio virus envelope E polypeptide comprises an amino acid sequence of SEQ ID NO:69, or an antigenic fragment or a variant thereof. In some embodiments, the modified Rocio virus envelope E polypeptide comprises an amino acid sequence at least 90% identical to SEQ ID NO:69. In some embodiments, the modified Rocio virus envelope E polypeptide is encoded by a DNA sequence comprising SEQ ID NO:70. In some embodiments, the modified Rocio virus envelope E polypeptide is encoded by an RNA sequence comprising SEQ ID NO:71.

In some embodiments, the flavivirus is a Spondweni virus. In some embodiments, the Spondweni virus parental envelope E polypeptide sequence (lacking the stem and anchor domains) comprises an amino acid sequence of SEQ ID NO:72 (see GenBank Accession No.: YP_009227187). In some embodiments, the modified Spondweni virus envelope E polypeptide comprises cysteine residues present at amino acid position numbers 5 and 102 with reference to SEQ ID NO:72. In some embodiments, the modified Spondweni virus envelope E polypeptide comprises an amino acid sequence of SEQ ID NO:73, or an antigenic fragment or a variant thereof. In some embodiments, the modified Spondweni virus envelope E polypeptide comprises an amino acid sequence at least 90% identical to SEQ ID NO:73. In some embodiments, the modified Spondweni envelope E polypeptide is encoded by a DNA sequence comprising SEQ ID NO:74. In some embodiments, the modified Spondweni virus envelope E polypeptide is encoded by an RNA sequence comprising SEQ ID NO:75.

In some embodiments, the polypeptide dimer consists of two modified flavivirus envelope E polypeptides that have the same amino acid sequence, i.e., the dimer is a homodimer. In some embodiments, the dimer is a heterodimer, wherein the two modified Zika virus envelope E polypeptides that comprise the dimer have different amino acid sequences. In some embodiments, the heterodimer is comprised of polypeptides that are at least 90%, 91%, 92%, 93%, 94%, 95%, 96%, 97%, 98%, or 99% identical.

In some embodiments, the cysteine residue in the fusion loop epitope of ectodomain DII on one polypeptide chain of the dimer forms an interchain disulfide bond with the cysteine residue in the ectodomain DI on the other polypeptide chain of the dimer.

In some embodiments, the modified flavivirus envelope E polypeptide is a soluble polypeptide that comprises ectodomains DI, DII, and DIII, and lacks stem and anchor domains. In some embodiments, the cysteine residues are present at amino acid position numbers 5 and 102 with reference to SEQ ID NOS:1, 7, 20, 24, 28, 32, 36, 40, 44, 48, 52, 56, 60, 64, 68, and 72.

In some embodiments, the modified flavivirus virus envelope E polypeptide comprises an amino acid sequence selected from the group consisting of SEQ ID NOS:2, 8, 17, 21, 25, 29, 33, 37, 41, 45, 49, 53, 57, 61, 65, 69, and 73.

In some embodiments, the modified flavivirus envelope E polypeptide comprises an amino acid sequence at least 80%, 85%, 90%, 91%, 92%, 93%, 94%, 95%, 96%, 97%, 98%, or 99% identical to any of SEQ ID NOS:2, 8, 17, 21, 25, 29, 33, 37, 41, 45, 49, 53, 57, 61, 65, 69, or 73. These can include fragments of any of SEQ ID NOS:2, 8, 17, 21, 25, 29, 33, 37, 41, 45, 49, 53, 57, 61, 65, 69, or 73, as well as substitution mutations.

An antigenic fragment is a polypeptide having an amino acid sequence that entirely is the same as part but not all of the amino acid sequence of one of the modified flavivirus envelope polypeptides. The antigenic fragment can be “free-standing,” or comprised within a larger polypeptide of which they form a part or region, most preferably as a single continuous region.

In some embodiments, the antigenic fragments include, for example, truncation polypeptides having the amino acid sequence of the polypeptides, except for deletion of a continuous series of residues that includes the amino terminus, or a continuous series of residues that includes the carboxyl terminus or deletion of two continuous series of residues, one including the amino terminus and one including the carboxyl terminus. In some embodiments, fragments are characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, and high antigenic index regions.

The fragment can be of any size. An antigenic fragment is capable of inducing an immune response in a subject or be recognized by a specific antibody. In some embodiments, the fragment corresponds to carboxyl-terminal truncation mutant. In some embodiments, the number of carboxyl terminal amino acids missing from the fragment ranges from 1-100 amino acids. In some embodiments, it ranges from 1-75 amino acids, 1-50 amino acids, 1-40 amino acids, 1-30 amino acids, 1-25 amino acids, 1-20 amino acids, 1-15 amino acids, 1-10 amino acids and 1-5 amino acids.

In some embodiments, the fragment is at least 50 amino acids, 100 amino acids, 150 amino acids, 200 amino acids or at least 250 amino acids in length. Of course, larger antigenic fragments are also useful according to the present invention, as are fragments corresponding to most, if not all, of the amino acid sequence of the polypeptides described herein.

In some embodiments, the modified flavivirus envelope E polypeptides have an amino acid sequence at least 80, 85%, 90%, 91%, 92%, 93%, 94%, 95%, 96%, 97%, 98%, 99%, or 100% identical to any of SEQ ID NOS:2, 8, 17, 21, 25, 29, 33, 37, 41, 45, 49, 53, 57, 61, 65, 69, or 73, or antigenic fragments thereof. In some embodiments, the variants are those that vary from the reference by conservative amino acid substitutions, i.e., those that substitute a residue with another of like characteristics. Typical substitutions are among Ala, Val, Leu and Ile; among Ser and Thr; among the acidic residues Asp and Glu; among Asn and Gln; and among the basic residues Lys and Arg, or aromatic residues Phe and Tyr. In some embodiments, the polypeptides are variants in which several, 5 to 10, 1 to 5, or 1 to 2 amino acids are substituted, deleted, or added in any combination.

In some embodiments, the polypeptide, variant or antigenic fragment thereof comprises a cleavable protein sequence and/or one or more affinity tags to aid in purification. In some embodiments, the affinity tag comprises at least 6 histidine residues (SEQ ID NO:12). In some embodiments, the affinity tag is a myc tag (SEQ ID NO:11). In some embodiments, the polypeptide comprises a myc tag (e.g., in the N-terminal portion of the polypeptide) and a histidine tag (e.g., in the C-terminal portion of the polypeptide). In some embodiments, the polypeptide or antigenic fragment thereof comprises a secretion signal to facilitate secretion of the protein through plasma membrane. In some embodiments, the secretion signal is a lysozyme secretion signal. In some embodiments, the signal peptide comprises SEQ ID NO: 13 and is encoded by SEQ ID NO: 14.

In some embodiments, the polypeptide comprises one or more linker motifs. In some embodiments, the linker motif can link heterologous sequences, such as an epitope tag with the envelope polypeptide sequences. In some embodiments, the linker motif comprises sequences selected from (G₄S)₁, (GGGS), (G₄S)₂, and (GGGSGGGS) (SEQ ID NO:76).

In some embodiments, the modified West Nile virus envelope E polypeptide comprises a myc tag, a 6×-histidine tag, and linker motifs and comprises an amino acid sequence of SEQ ID NO:17, or an antigenic fragment or a variant thereof. In some embodiments, the modified West Nile virus envelope E polypeptide is encoded by a DNA sequence comprising SEQ ID NO: 18. In some embodiments, the modified West Nile virus envelope E polypeptide is encoded by an RNA sequence comprising SEQ ID NO: 19.

In terms of variants that are immunologically functional equivalents, it is well understood by the skilled artisan that, inherent in the definition is the concept that there is a limit to the number of changes that may be made within a defined portion of the molecule and still result in a molecule with an acceptable level of equivalent immunological activity. An immunologically functional equivalent peptide or polypeptide are thus defined herein as those peptide(s) or polypeptide(s) in which certain, not most or all, of the amino acid(s) may be substituted.

In particular, where a shorter length peptide is concerned, it is contemplated that fewer amino acid substitutions should be made within the given peptide. A longer polypeptide may have an intermediate number of changes. The full-length protein will have the most tolerance for a larger number of changes. Of course, a plurality of distinct polypeptides/peptides with different substitutions may easily be made and used in accordance with the invention.

It also is well understood that where certain residues are shown to be particularly important to the immunological or structural properties of a protein or peptide, e.g., residues in binding regions or active sites, such residues may not generally be exchanged. This is an important consideration in the present invention, where changes in the antigenic site should be carefully considered and subsequently tested to ensure maintenance of immunological function (e.g., antigenicity), where maintenance of immunological function is desired. In this manner, functional equivalents are defined herein as those peptides or polypeptides which maintain a substantial amount of their native immunological activity.

Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as immunologically functional equivalents.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein, polypeptide or peptide is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It also is understood in the art that the substitution of like amino acids can be made effectively on the basis of hydrophilicity, particularly where the immunological functional equivalent polypeptide or peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a immunological property of the protein.

As detailed in U.S. Pat. No. 4,554,101, the following hydrophilicity values have been assigned to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

Numerous scientific publications have also been devoted to the prediction of secondary structure, and to the identification of an epitope, from analyses of an amino acid sequence (Chou & Fasman, 1974a,b; 1978a,b, 1979). Any of these may be used, if desired, to supplement the teachings of U.S. Pat. No. 4,554,101.

Moreover, computer programs are currently available to assist with predicting an antigenic portion and an epitopic core region of one or more proteins. polypeptides or peptides. Examples include those programs based upon the Jameson-Wolf analysis (Jameson & Wolf, 1988; Wolf et al., 1988), the program PepPlot (Brutlag et al., 1990; Weinberger et al., 1985), and other new programs for protein tertiary structure prediction (Fetrow & Bryant, 1993). Another commercially available software program capable of carrying out such analyses is MacVector (IBI, New Haven, Conn.).

In further embodiments, major antigenic determinants of a peptide or polypeptide may be identified by an empirical approach in which portions of a nucleic acid encoding a peptide or polypeptide are expressed in a recombinant host, and the resulting peptide(s) or polypeptide(s) tested for their ability to elicit an immune response. For example, PCR can be used to prepare a range of peptides or polypeptides lacking successively longer fragments of the C-terminus of the amino acid sequence. The immunoactivity of each of these peptides or polypeptides is determined to identify those fragments or domains that are immunodominant. Further studies in which only a small number of amino acids are removed at each iteration then allows the location of the antigenic determinant(s) of the peptide or polypeptide to be more precisely determined.

Another method for determining a major antigenic determinant of a peptide or polypeptide is the SPOTs system (Genosys Biotechnologies, Inc., The Woodlands, Tex.). In this method, overlapping peptides are synthesized on a cellulose membrane, which following synthesis and deprotection, is screened using a polyclonal or monoclonal antibody. An antigenic determinant of the peptides or polypeptides which are initially identified can be further localized by performing subsequent syntheses of smaller peptides with larger overlaps, and by eventually replacing individual amino acids at each position along the immunoreactive sequence.

Once one or more such analyses are completed, an antigenic composition, such as for example a peptide or a polypeptide is prepared that contain at least the essential features of one or more antigenic determinants. An antigenic composition is then employed in the generation of antisera against the composition, and preferably the antigenic determinant(s).

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid. Nucleic acids encoding these antigenic compositions also can be constructed and inserted into one or more expression vectors by standard methods (Sambrook et al., 1987), for example, using PCR cloning methodology. In addition to the polypeptides described herein, sterically similar polypeptides may be formulated to mimic the key portions of the peptide or polypeptide structure or to interact specifically with, for example, an antibody. Such compounds, which may be termed peptidomimetics, may be used in the same manner as a peptide or polypeptide of the invention and hence are also immunologically functional equivalents.

Certain mimetics that mimic elements of protein secondary structure have been described. The underlying rationale behind the use of peptide mimetics is that the peptide backbone of proteins exists chiefly to orientate amino acid side chains in such a way as to facilitate molecular interactions, such as those of antibody and antigen. A peptide mimetic is thus designed to permit molecular interactions similar to the natural molecule.

In particular embodiments, an antigenic composition is mutated for purposes such as, for example, enhancing its immunogenicity or producing or identifying a immunologically functional equivalent sequence. Methods of mutagenesis are well known to those of skill in the art (Sambrook et al., 1987).

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In some embodiments, site directed mutagenesis is used. Site-specific mutagenesis is a technique useful in the preparation of an antigenic composition, through specific mutagenesis of the underlying DNA. In general, the technique of site-specific mutagenesis is well known in the art. The technique further provides a ready ability to prepare and test sequence variants, incorporating one or more of the foregoing considerations, by introducing one or more nucleotide sequence changes into the DNA. Site-specific mutagenesis allows the production of a mutant through the use of specific oligonucleotide sequence(s) which encode the DNA sequence of the desired mutation, as well as a sufficient number of adjacent nucleotides, to provide a primer sequence of sufficient size and sequence complexity to form a stable duplex on both sides of the position being mutated. Typically, a primer of about 17 to about 75 nucleotides in length is preferred, with about 10 to about 25 or more residues on both sides of the position being altered, while primers of about 17 to about 25 nucleotides in length being more preferred, with about 5 to 10 residues on both sides of the position being altered.

In general, site-directed mutagenesis is performed by first obtaining a single-stranded vector, or melting of two strands of a double stranded vector which includes within its sequence a DNA sequence encoding the desired protein. As will be appreciated by one of ordinary skill in the art, the technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

This mutagenic primer is then annealed with the single-stranded DNA preparation, and subjected to DNA polymerizing enzymes such as, for example, E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

Alternatively, a pair of primers may be annealed to two separate strands of a double stranded vector to simultaneously synthesize both corresponding complementary strands with the desired mutation(s) in a PCR reaction. A genetic selection scheme to enrich for clones incorporating the mutagenic oligonucleotide has been devised (Kunkel et al., 1987). Alternatively, the use of PCR with commercially available thermostable enzymes such as Taq polymerase may be used to incorporate a mutagenic oligonucleotide primer into an amplified DNA fragment that can then be cloned into an appropriate cloning or expression vector (Tomic et al., 1990; Upender et al., 1995). A PCR employing a thermostable ligase in addition to a thermostable polymerase also may be used to incorporate a phosphorylated mutagenic oligonucleotide into an amplified DNA fragment that may then be cloned into an appropriate cloning or expression vector (Michael 1994).

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

Additionally, one particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation (Cunningham et al., 1989).

### Nucleic Acids and Viral Vectors

In another aspect, the invention provides a nucleic acid encoding a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide.

In some embodiments, the polypeptides are encoded by polynucleotides that are optimized for high level expression in cells, such as insect cells, bacterial cells, or mammalian cells.

In some embodiments, the modified flavivirus envelope E polypeptides are encoded by a DNA comprising a sequence at least about 70%, 75%, 80%, 85%, 90%, 91%, 92%, 93%, 94%, 95%, 96%, 97%, 98%, 99%, or 100% identical to any of SEQ ID NOS:4, 9, 18, 22, 26, 30, 34, 38, 42, 46, 50, 54, 58, 62, 66, 70, or 74.

In some embodiments, the modified flavivirus envelope E polypeptides are encoded by an RNA comprising a sequence at least about 70%, 75%, 80%, 85%, 90%, 91%, 92%, 93%, 94%, 95%, 96%, 97%, 98%, 99%, or 100% identical to any of SEQ ID NOS:6, 10, 19, 23, 27, 31, 35, 39, 43, 47, 51, 55, 59, 63, 67, 71, or 75.

In some embodiments, the invention provides a nucleic acid that encodes any of SEQ ID NOS:2, 8, 17, 21, 25, 29, 33, 37, 41, 45, 49, 53, 57, 61, 65, 69, or 73, or an antigenic fragment thereof, or a polypeptide sequence that is at least 80%, 85%, 90%, 91%, 92%, 93%, 94%, 95%, 96%, 97%, 98%, or 99% identical to any of SEQ ID NOS:2, 8, 17, 21, 25, 29, 33, 37, 41, 45, 49, 53, 57, 61, 65, 69, or 73. In some embodiments, the nucleic acid comprises any of SEQ ID NOS:4, 9, 18, 22, 26, 30, 34, 38, 42, 46, 50, 54, 58, 62, 66, 70, or 74 or a nucleic acid that is at least 50%, 55%, 60%, 65%, 70%, 75% 80%, 85%, 90%, 91%, 92%, 93%, 94%, 95%, 96%, 97%, 98%, or 99% identical thereto.

In some embodiments, the nucleic acid is an mRNA. In some embodiments, the nucleic acid comprises any of SEQ ID NOS:6, 10, 19, 23, 27, 31, 35, 39, 43, 47, 51, 55, 59, 63, 67, 71, or 75. or a nucleic acid that is at least 50%, 55%, 60%, 65%, 70%, 75% 80%, 85%, 90%, 91%, 92%, 93%, 94%, 95%, 96%, 97%, 98%, or 99% identical thereto.

In some embodiments, the nucleic acid is encoded by a viral vector. In some embodiments, the viral vector comprises a nucleic acid sequence encoding the modified flavivirus envelope E protein. In some embodiments, the modified flavivirus envelope E protein is fused to an epitope tag. The epitope tag is not limiting, and in some embodiments is selected from the group consisting of Myc, FLAG, hemagglutinin (HA) and/or combinations thereof.

The viral vector is not limiting. In some embodiments, the viral vector will typically comprise a highly attenuated, non-replicative virus. Viral vectors include, but are not limited to, DNA viral vectors such as those based on adenoviruses, herpes simplex virus, avian viruses, such as Newcastle disease virus, poxviruses such as vaccinia virus, and parvoviruses, including adeno-associated virus; and RNA viral vectors, including, but not limited to, the retroviral vectors. Vaccinia vectors and methods useful in immunization protocols are described in U.S. Pat. No. 4,722,848. Retroviral vectors include murine leukemia virus, and lentiviruses such as human immunodeficiency virus. Naldini et al. (1996) Science 272:263-267. Replication-defective retroviral vectors harboring a nucleotide sequence of interest as part of the retroviral genome can be used. Such vectors have been described in detail. (Miller et al. (1990) Mol. Cell. Biol. 10:4239; Kolberg, R. (1992) J. NIH Res. 4:43; Cornetta et al. (1991) Hum. Gene Therapy 2:215).

Adenovirus and adeno-associated virus vectors useful in the invention may be produced according to methods already taught in the art. (See, e.g., Karlsson et al. (1986) EMBO 5:2377; Carter (1992) Current Opinion in Biotechnology 3:533-539; Muzcyzka (1992) Current Top. Microbiol. Immunol. 158:97-129; Gene Targeting: A Practical Approach (1992) ed. A. L. Joyner, Oxford University Press, NY). Several different approaches are feasible.

Alpha virus vectors, such as Venezuelan Equine Encephalitis (VEE) virus, Semliki Forest virus (SFV) and Sindbis virus vectors, can be used for efficient gene delivery. Replication-deficient vectors are available. Such vectors can be administered through any of a variety of means known in the art, such as, for example, intranasally or intratumorally. See Lundstrom, Curr. Gene Ther. 2001 1:19-29.

Additional literature describing viral vectors which could be used in the compositions and methods of the present invention include the following: Horwitz, M. S., Adenoviridae and Their Replication, in Fields, B., et al. (eds.) Virology, Vol. 2, Raven Press New York, pp. 1679-1721, 1990); Graham, F. et al., pp. 109-128 in Methods in Molecular Biology, Vol. 7: Gene Transfer and Expression Protocols, Murray, E. (ed.), Humana Press, Clifton, N.J. (1991); Miller, et al. (1995) FASEB Journal 9:190-199, Schreier (1994) Pharmaceutica Acta Helvetiae 68:145-159; Schneider and French (1993) Circulation 88:1937-1942; Curiel, ct al. (1992) Human Gene Therapy 3:147-154; WO 95/00655; WO 95/16772; WO 95/23867; WO 94/26914; WO 95/02697 (Jan. 26, 1995); and WO 95/25071.

In some embodiments, the viral vector is a retrovirus/lentivirus, adenovirus, adeno-associated virus, alpha virus, vaccinia virus or a herpes simplex virus. In some embodiments, the viral vector is a lentiviral vector.

### Expression Vectors, Host Cells, and Recombinant Expression

The present invention also relates to vectors that comprise the nucleic acids of the present invention, including cloning vectors and expression vectors, host cells which harbor vectors of the invention, or are engineered to express the polypeptides, and methods for the production of polypeptides of the invention by recombinant techniques. Cell-free translation systems can also be employed to produce such proteins using RNAs derived from the DNA constructs of the invention.

Representative examples of appropriate hosts include bacterial cells, such as streptococci, staphylococci, Escherichia coli, Streptomyces and Bacillus subtilis; fungal cells, such as yeast and Aspergillus; insect cells such as Drosophila S2 and Spodoptera Sf9; mammalian cells such as CHO, COS, HeLa, C127, 3T3, BHK, HEK-293 and Bowes melanoma. A great variety of expression systems can be used, including DNA or RNA vectors.

In other embodiments, this invention provides an isolated nucleic acid molecules of the invention operably linked to a heterologous promoter. The invention further provides an isolated nucleic acid molecule operably linked to a heterologous promoter, wherein said isolated nucleic acid molecule is capable of expressing a modified flavivirus envelope E protein or an antigenic fragment or derivative thereof when used to transform an appropriate host cell.

In another embodiment, the invention provides a host cell comprising a nucleic acid encoding a modified flavivirus envelope E polypeptide.

Methods for the production of polypeptides of the invention including culturing a host cells transfected with one or more of the vectors of the present invention under conditions promoting expression of the polypeptide encoded by the vector, and isolating the polypeptide so expressed from the cell culture.

Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MAXBAC 2.0 from INVITROGEN and BACPACK baculovirus expression system from CLONTECH.

Other examples of expression systems include COMPLETE CONTROL Inducible Mammalian Expression System from STRATAGENE, which involves a synthetic ecdysone-inducible receptor, or its pET Expression System, an E. coli expression system. Another example of an inducible expression system is available from INVITROGEN, which carries the T-REX (tetracycline-regulated expression) System, an inducible mammalian expression system that uses the full-length CMV promoter. INVITROGEN also provides a yeast expression system called the Pichia methanolica Expression System, which is designed for high-level production of recombinant proteins in the methylotrophic yeast P. methanolica. One of skill in the art would know how to manipulate a vector, such as an expression construct, to produce a nucleic acid sequence or its cognate polypeptide, protein, or peptide.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment involves the use of gene transfer to immortalize cells for the production of proteins. The nucleic acid for the protein of interest may be transferred as described above into appropriate host cells followed by culture of cells under the appropriate conditions.

Examples of useful mammalian host cell lines are Vero and HeLa cells and cell lines of Chinese hamster ovary, W138, BHK, COS-7, HEK-293, HcpG2, NIH3T3, RIN and MDCK cells. In addition, a host cell clone may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used, including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to methotrexate; gpt, that confers resistance to mycophenolic acid; neo, that confers resistance to the aminoglycoside G418; and hygro, that confers resistance to hygromycin.

As used herein, the terms “cell,” “cell line,” and “cell culture” may be used interchangeably. All of these terms also include their progeny, which are any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes (e.g., bacteria or yeast), depending upon whether the desired result is replication of the vector or expression of part or all of the vector-encoded nucleic acid sequences. Numerous cell lines and cultures are available for use as a host cell, and they can be obtained through the American Type Culture Collection (ATCC). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5(a. JM109, and KCB, as well as a number of commercially available bacterial hosts such as SURE Competent Cells and SOLOPACK Gold Cells (STRATAGENE, La Jolla). Alternatively, bacterial cells such as E. coli LE392 could be used as host cells for phage viruses.

Examples of eukaryotic host cells for replication and/or expression of a vector include HeLa, NIH3T3, Jurkat, HEK-293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### Pharmaceutical Compositions

In some embodiments, the invention provides a pharmaceutical composition comprising an immunologically-effective amount of a polypeptide or a polypeptide dimer as described herein. In some embodiments, the pharmaceutical composition is a vaccine composition.

In some embodiments, the pharmaceutical composition comprises an immunologically-effective amount of a nucleic acid encoding a polypeptide or an antigenic fragment or variant thereof, or a vector encoding the same as described herein. In some embodiments, the nucleic acid is a DNA. In some embodiments, the nucleic acid is an mRNA.

In some embodiments, the pharmaceutical composition comprises an effective amount of a polypeptide or polypeptide dimer comprising a modified flavivirus envelope E polypeptide, wherein the modified Zika virus envelope E polypeptide comprises i) a cysteine residue in a fusion loop epitope of ectodomain DII; and ii) a cysteine residue in ectodomain DI located in an N-terminal portion of the polypeptide.

In another aspect, the invention provides a pharmaceutical composition comprising an effective amount of a nucleic acid or an effective amount of a vector encoding a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide.

In some embodiments, the nucleic acid is formulated as a nanoparticle. In some embodiments, the nanoparticle is a lipid nanoparticle. See, e.g., U.S. Pat. No. 10,646,549 for disclosures of various nanoparticle formulations for nucleic acids, including lipid or lipid-like nanoparticles.

In some embodiments, the compositions are administered as pharmaceutical compositions and induce an immune response to the polypeptide dimer in a cell, tissue or animal (e.g., a human). As used herein, a “pharmaceutical composition” (which alternatively may be referred to as an “immunizing composition” or an “antigenic composition”) may comprise an antigen (e.g., a protein, peptide, or polypeptide). In some embodiments, the pharmaceutical composition comprises a nucleic acid or vector encoding a polypeptide antigen.

In some embodiments, the immunogenic composition or vaccine comprises at least one adjuvant. In some embodiments, the adjuvant is a polyphosphazene adjuvant. In some embodiments, the adjuvant is selected from PCPP, PCEP and combinations thereof. In some embodiments, the composition further comprises a TLR7/8 agonist. In some embodiments, the polypeptide dimer, adjuvant and TLR7/8 agonist are in a complex. In some embodiments, the TLR7/8 agonist is R-848.

In another non-limiting example, a vaccine or immunogenic composition may comprise a saponin and a lipid. A vaccine or immunizing composition of the present disclosure, and its various components, may be prepared by any method disclosed herein or as would be known to one of ordinary skill in the art, in light of the present disclosure.

In some embodiments, the antigenic composition is in a mixture that comprises an additional immunostimulatory agent or nucleic acids encoding such an agent.

Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises an HLA anchor motif amino acids.

In certain embodiments, an antigenic or pharmaceutical composition can be used as an effective vaccine in inducing an anti-flavivirus humoral and/or cell-mediated immune response in an animal, including a human. In some embodiments, the immune response is specific to the particular species or strain of flavivirus the modified envelope E polypeptide is derived from. The present invention contemplates one or more antigenic compositions or vaccines for use in both active and passive immunization embodiments.

A vaccine or immunizing composition of the present invention may vary in its composition of proteinaceous components. It will be understood that various compositions described herein may further comprise additional components. For example, one or more vaccine or immunogenic composition components may be comprised in a lipid, lipid-like molecule(s), or liposome.

It is understood that an immunizing composition may be made by a method that is well known in the art, including but not limited to chemical synthesis by solid phase synthesis and purification away from the other products of the chemical reactions by HPLC, or production by the expression of a nucleic acid sequence (e.g., a DNA sequence) encoding a peptide or polypeptide comprising an antigen of the present invention in an in vitro translation system or in a living cell including, for example, in a yeast cell, bacterial, mammalian cells or baculovirus/insect cells. The antigenic composition may be isolated and extensively purified to remove one or more undesired small molecular weight molecules and/or lyophilized for more ready formulation into a desired vehicle. It is further understood that amino acid additions, deletions, mutations, chemical modification and such like that are made in an antigenic composition component, such as a vaccine, will preferably not substantially interfere with the antibody recognition of the epitopic sequence.

In some embodiments, the polypeptides may be made by methods known to those of ordinary skill in the art, such as, for example, peptide synthesis using automated peptide synthesis machines, such as those available from Applied Biosystems (Foster City, Calif.).

In some embodiments, longer peptides or polypeptides also may be prepared, e.g., by recombinant means. In certain embodiments, a nucleic acid encoding a polypeptide, for example, to produce an antigenic composition in vitro or in vivo for the various compositions and methods of the present invention. For example, in certain embodiments, a nucleic acid encoding an antigen is comprised in, for example, a vector in a recombinant cell. The nucleic acid may be expressed to produce a peptide or polypeptide comprising an antigenic sequence. The peptide or polypeptide may be secreted from the cell or comprised as part of or within the cell.

In some embodiments, the antigen may be expressed using a vector such as a viral vector. For example, in certain embodiments, the coding sequence could be inserted in a viral vector, including but not limited to an adenovirus, adeno-associated virus, measles virus, poxvirus, herpes complex, retrovirus, lentivirus, alphavirus, flavivirus, rabdovirus, Newcastle disease virus and picronavirus.

As modifications and changes may be made in the structure of an antigenic composition of the present disclosure, and still obtain molecules having like or otherwise desirable characteristics, such immunologically functional equivalents are also encompassed within the present invention.

For example, certain amino acids may be substituted for other amino acids in a peptide, polypeptide or protein structure without appreciable loss of interactive binding capacity with structures such as, for example, antigen-binding regions of antibodies, binding sites on substrate molecules or receptors, or such like. Since it is the interactive capacity and nature of a peptide, polypeptide or protein that defines its biological (e.g., immunological) functional activity, certain amino acid sequence substitutions can be made in an amino acid sequence (or, of course. its underlying DNA coding sequence) and nevertheless obtain a peptide or polypeptide with like (agonistic) properties. It is thus contemplated by the inventors that various changes may be made in the sequence of the polypeptide without appreciable loss of biological utility or activity. In particular cases, one or more of the potential glycosylation sites is mutated or deleted and in particular embodiments there is also one or more other amino acids that are modified compared to the corresponding wild-type sequence.

As used herein, an “amino molecule” refers to any amino acid, amino acid derivative or amino acid mimic as would be known to one of ordinary skill in the art. In certain embodiments, the residues of the antigenic composition comprises amino molecules that are sequential, without any non-amino molecule interrupting the sequence of amino molecule residues. In other embodiments, the sequence may comprise one or more non-amino molecule moieties. In particular embodiments, the sequence of residues of the antigenic composition may be interrupted by one or more non-amino molecule moieties.

In some embodiments, antigenic compositions may encompass an amino molecule sequence comprising at least one of the 20 common amino acids in naturally synthesized proteins, or at least one modified or unusual amino acid.

In a further embodiment of the invention, one or more vaccine or immunizing composition components may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers.

In any case, a vaccine component (e.g., an antigenic peptide or polypeptide) may be isolated and/or purified from the chemical synthesis reagents, cell or cellular components. In a method of producing the vaccine or immunogenic composition component, purification is accomplished by any appropriate technique that is described herein or well-known to those of skill in the art (e.g., Sambrook et al., 1987). There is no general requirement that an antigenic composition of the present invention or other vaccine component always be provided in their most purified state. Indeed, it is contemplated that less substantially purified vaccine or immunogenic composition component, which is nonetheless enriched in the desired compound, relative to the natural state, will have utility in certain embodiments, such as, for example, total recovery of protein product, or in maintaining the activity of an expressed protein. However, it is contemplated that inactive products also have utility in certain embodiments, such as, e.g., in determining antigenicity via antibody generation.

The present invention also provides purified, and in certain embodiments, substantially purified vaccines or immunogenic composition components. The term “purified vaccine component” or “purified immunogenic composition component” as used herein, is intended to refer to at least one respective vaccine or immunogenic composition component (e.g., a proteinaceous composition, isolatable from cells), wherein the component is purified to any degree relative to its naturally-obtainable state, e.g., relative to its purity within a cellular extract or reagents of chemical synthesis. In certain aspects wherein the vaccine component is a proteinaceous composition, a purified vaccine component also refers to a wild-type or mutant protein, polypeptide, or peptide free from the environment in which it naturally occurs.

Where the term “substantially purified” is used, this will refer to a composition in which the specific compound (e.g., a protein, polypeptide, or peptide) forms the major component of the composition, such as constituting about 50% of the compounds in the composition or more. In preferred embodiments, a substantially purified vaccine component will constitute more than about 60%, about 70%, about 80%, about 90%, about 95%, about 99% or even more of the compounds in the composition.

In certain embodiments, a vaccine or immunogenic composition component may be purified to homogeneity. As applied to the present invention, “purified to homogeneity,” means that the vaccine component has a level of purity where the compound is substantially free from other chemicals, biomolecules or cells. For example, a purified peptide, polypeptide or protein will often be sufficiently free of other protein components so that degradative sequencing may be performed successfully. Various methods for quantifying the degree of purification of a vaccine component will be known to those of skill in the art in light of the present disclosure. These include, for example, determining the specific protein activity of a fraction (e.g., antigenicity), or assessing the number of polypeptides within a fraction by gel electrophoresis.

Various techniques suitable for use in chemical, biomolecule or biological purification, well known to those of skill in the art, may be applicable to preparation of a vaccine component of the present invention. These include, for example, precipitation with ammonium sulfate, PEG, antibodies and the like or by heat denaturation, followed by centrifugation; fractionation, chromatographic procedures, including but not limited to, partition chromatograph (e.g., paper chromatograph, thin-layer chromatograph (TLC), gas-liquid chromatography and gel chromatography) gas chromatography, high performance liquid chromatography, affinity chromatography, supercritical flow chromatography ion exchange, gel filtration, reverse phase, hydroxylapatite, lectin affinity; isoelectric focusing and gel electrophoresis.

In certain aspects, a nucleic acid may be purified on polyacrylamide gels, and/or cesium chloride centrifugation gradients, or by any other means known to one of ordinary skill in the art (see for example, Sambrook et al. 1989, incorporated herein by reference). In further aspects, a purification of a proteinaceous sequence may be conducted by recombinantly expressing the sequence as a fusion protein. Such purification methods are routine in the art. This is exemplified by the generation of an specific protein-glutathione S-transferase fusion protein, expression in E. coli, and isolation to homogeneity using affinity chromatography on glutathione-agarose or the generation of a polyhistidine tag on the N- or C-terminus of the protein, and subsequent purification using Ni-affinity chromatography. In particular aspects, cells or other components of the vaccine may be purified by flow cytometry. Flow cytometry involves the separation of cells or other particles in a liquid sample. and is well known in the art (see, for example, U.S. Pat. Nos. 3,826,364, 4,284,412, 4,989,977, 4,498,766, 5,478,722, 4,857,451, 4,774,189, 4,767,206, 4,714,682, 5,160,974 and 4,661,913). Any of these techniques described herein, and combinations of these and any other techniques known to skilled artisans, may be used to purify and/or assay the purity of the various chemicals, proteinaceous compounds, nucleic acids, cellular materials and/or cells that may comprise a vaccine of the present invention. As is generally known in the art, it is believed that the order of conducting the various purification steps may be changed, or that certain steps may be omitted, and still result in a suitable method for the preparation of a substantially purified antigen or other vaccine component.

It is contemplated that an antigenic composition of the invention may be combined with one or more additional components to form a more effective composition or vaccine. Non-limiting examples of additional components include, for example, one or more additional antigens, immunomodulators or adjuvants to stimulate an immune response to an antigenic composition of the present invention and/or the additional component(s).

For example, in some embodiments one or more immunomodulators can be included in the vaccine to augment a cell's or a patient's (e.g., an animal's) response. Immunomodulators can be included as purified proteins, nucleic acids encoding immunomodulators, and/or cells that express immunomodulators in the vaccine composition, for example. The following sections list non-limiting examples of immunomodulators that are of interest, and it is contemplated that various combinations of immunomodulators may be used in certain embodiments (e.g., a cytokine and a chemokine).

Interleukins, cytokines, nucleic acids encoding interleukins or cytokines, and/or cells expressing such compounds are contemplated as possible vaccine components. Interleukins and cytokines, include but are not limited to interleukin 1 (IL-1), IL-2, IL-3, IL-4, IL-5, IL-6, IL-7, IL-8, IL-9, IL-10, IL-11, IL-12, IL-13, IL-14, IL-15, IL-18, .beta.-interferon, α-interferon, γ-interferon, angiostatin, thrombospondin, endostatin, GM-CSF, G-CSF, M-CSF, METH-1, METH-2, tumor necrosis factor, TGFβ, LT and combinations thereof.

Chemokines, nucleic acids that encode for chemokines, and/or cells that express such also may be used as vaccine components. Chemokines generally act as chemoattractants to recruit immune effector cells to the site of chemokine expression. It may be advantageous to express a particular chemokine coding sequence in combination with, for example, a cytokine coding sequence, to enhance the recruitment of other immune system components to the site of treatment. Such chemokines include, for example, RANTES, MCAF, MIP1-alpha, MIP1-Beta, IP-10 and combinations thereof. The skilled artisan will recognize that certain cytokines are also known to have chemoattractant effects and could also be classified under the term chemokines.

In certain embodiments, an antigenic composition may be chemically coupled to a carrier or recombinantly expressed with an immunogenic carrier peptide or polypetide (e.g., an antigen-carrier fusion peptide or polypeptide) to enhance an immune reaction. Exemplary and preferred immunogenic carrier amino acid sequences include hepatitis B surface antigen, keyhole limpet hemocyanin (KLH) and bovine serum albumin (BSA). Other albumins such as ovalbumin, mouse serum albumin or rabbit serum albumin also can be used as immunogenic carrier proteins. Means for conjugating a polypeptide or peptide to an immunogenic carrier protein are well known in the art and include, for example, glutaraldehyde, m-maleimidobenzoyl-N-hydroxysuccinimide ester, carbodiimide and bis-biazotized benzidine.

It may be desirable to coadminister biologic response modifiers (BRM), which have been shown to upregulate T cell immunity or downregulate suppressor cell activity. Such BRMs include, but are not limited to. cimetidine (CIM; 1200 mg/d) (Smith/Kline. PA); low-dose cyclophosphamide (CYP; 300 mg/m²) (Johnson/Mead, NJ), or a gene encoding a protein involved in one or more immune helper functions, such as B-7.

Immunization protocols have used adjuvants to stimulate responses for many years, and as such adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation.

In one aspect, an adjuvant effect is achieved by use of an agent, such as alum, used in about 0.05 to about 0.1% solution in phosphate buffered saline. Alternatively, the antigen is made as an admixture with synthetic polymers of sugars (Carbopol) used as an about 0.25% solution. Adjuvant effect may also be made my aggregation of the antigen in the vaccine by heat treatment with temperatures ranging between about 70 degrees to about 101 degrees C. for a 30-second to 2-minute period, respectively. Aggregation by reactivating with pepsin treated (Fab) antibodies to albumin, mixture with bacterial cell(s) such as C. parvum, an endotoxin or a lipopolysaccharide component of Gram-negative bacteria, emulsion in physiologically acceptable oil vehicles, such as mannide mono-oleate (Aracel A), or emulsion with a 20% solution of a perfluorocarbon (Fluosol-DA) used as a block substitute, also may be employed.

Some adjuvants, for example, certain organic molecules obtained from bacteria, act on the host rather than on the antigen. An example is muramyl dipeptide (N-acetylmuramyl-L-alanyl-D-isoglutamine [MDP]), a bacterial peptidoglycan. The effects of MDP, as with most adjuvants, are not fully understood. MDP stimulates macrophages but also appears to stimulate B cells directly. The effects of adjuvants, therefore, are not antigen-specific. If they are administered together with a purified antigen, however, they can be used to selectively promote the response to the antigen.

Adjuvants have been used experimentally to promote a generalized increase in immunity against unknown antigens (e.g., U.S. Pat. No. 4,877,611). In certain embodiments, hemocyanins and hemoerythrins may also be used in the invention. The use of hemocyanin from keyhole limpet (KLH) is preferred in certain embodiments, although other molluscan and arthropod hemocyanins and hemoerythrins may be employed.

Another group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative MTPPE, are also contemplated.

U.S. Pat. No. 4,950,645 describes a lipophilic disaccharide-tripeptide derivative of muramyl dipeptide which is described for use in artificial liposomes formed from phosphatidyl choline and phosphatidyl glycerol. It is the to be effective in activating human monocytes and destroying tumor cells, but is non-toxic in generally high doses. The compounds of U.S. Pat. No. 4,950,645 and PCT Patent Application WO 91/16347, are contemplated for use with cellular carriers and other embodiments of the present invention.

Another adjuvant contemplated for use in the present invention is BCG. BCG (bacillus Calmette-Guerin, an attenuated strain of Mycobacterium) and BCG-cell wall skeleton (CWS) may also be used as adjuvants in the invention, with or without trehalose dimycolate. Trehalose dimycolate may be used itself. Trehalose dimycolate may be prepared as described in U.S. Pat. No. 4,579,945.

BCG is an important clinical tool because of its immunostimulatory properties. BCG acts to stimulate the reticulo-endothelial system, activates natural killer cells and increases proliferation of hematopoietic stem cells. Cell wall extracts of BCG have proven to have excellent immune adjuvant activity. Molecular genetic tools and methods for mycobacteria have provided the means to introduce foreign genes into BCG.

Live BCG is an effective and safe vaccine used worldwide to prevent tuberculosis. BCG and other mycobacteria are highly effective adjuvants, and the immune response to mycobacteria has been studied extensively. It is one of the few vaccines that can be given at birth, it engenders long-lived immune responses with only a single dose, and there is a worldwide distribution network with experience in BCG vaccination. An exemplary BCG vaccine is sold as TICE BCG (Organon Inc., West Orange, N.J.).

Amphipathic and surface active agents, e.g., saponin and derivatives such as QS21 (Cambridge Biotech), form yet another group of adjuvants for use with the immunogens of the present invention. Nonionic block copolymer surfactants may also be employed. Oligonucleotides are another useful group of adjuvants. Quil A and lentinen are other adjuvants that may be used in certain embodiments of the present invention.

In some embodiments, detoxified endotoxins can be used as adjuvants, such as the refined detoxified endotoxin of U.S. Pat. No. 4,866,034. These refined detoxified endotoxins are effective in producing adjuvant responses in mammals. Of course, the detoxified endotoxins may be combined with other adjuvants to prepare multi-adjuvant-incorporated cells. For example, combination of detoxified endotoxins with trehalose dimycolate is particularly contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cell wall skeleton (CWS) or CWS and trehalose dimycolate, as described in U.S. Pat. Nos. 4,436,727, 4,436,728 and 4,505,900. Combinations of just CWS and trehalose dimycolate, without detoxified endotoxins, is also envisioned to be useful, as described in U.S. Pat. No. 4,520,019.

In other embodiments, the present invention contemplates that a variety of adjuvants may be employed in the membranes of cells, resulting in an improved immunogenic composition. The only requirement is, generally, that the adjuvant be capable of incorporation into, physical association with, or conjugation to, the cell membrane of the cell in question. Those of skill in the art will know the different kinds of adjuvants that can be conjugated to cellular vaccines in accordance with this invention and these include alkyl lysophosphilipids (ALP); BCG; and biotin (including biotinylated derivatives) among others. Certain adjuvants particularly contemplated for use are the teichoic acids from Gram-cells. These include the lipoteichoic acids (LTA), ribitol teichoic acids (RTA) and glycerol teichoic acid (GTA). Active forms of their synthetic counterparts may also be employed in connection with the invention.

Various adjuvants, even those that are not commonly used in humans, may still be employed in animals, where, for example, one desires to raise antibodies or to subsequently obtain activated T cells. The toxicity or other adverse effects that may result from either the adjuvant or the cells, e.g., as may occur using non-irradiated tumor cells, is irrelevant in such circumstances.

One group of adjuvants preferred for use in some embodiments of the present invention are those that can be encoded by a nucleic acid (e.g., DNA or RNA). It is contemplated that such adjuvants may be encoded in a nucleic acid (e.g., an expression vector) encoding the antigen, or in a separate vector or other construct. These nucleic acids encoding the adjuvants can be delivered directly, such as for example with lipids or liposomes.

An antigenic composition of the present invention may be mixed with one or more additional components (e.g., excipients, salts, etc.) which are pharmaceutically acceptable and compatible with at least one active ingredient (e.g., antigen). Suitable excipients are, for example, water, saline, dextrose, glycerol, ethanol and combinations thereof.

An antigenic composition of the present invention may be formulated into the vaccine as a neutral or salt form. A pharmaceutically-acceptable salt, includes the acid addition salts (formed with the free amino groups of the peptide) and those which are formed with inorganic acids such as, for example, hydrochloric or phosphoric acid, or such organic acids as acetic, oxalic, tartaric, mandelic, and the like. A salt formed with a free carboxyl group also may be derived from an inorganic base such as, for example, sodium, potassium, ammonium, calcium, or ferric hydroxide, and such organic bases as isopropylamine, trimethylamine, 2-ethylamino ethanol, histidine, procaine, and combinations thereof.

In addition, if desired, an antigenic composition may comprise minor amounts of one or more auxiliary substances such as for example wetting or emulsifying agents, pH buffering agents, etc. which enhance the effectiveness of the antigenic composition or vaccine.

Once produced, synthesized and/or purified, an antigen or other vaccine component may be prepared as a vaccine or immunogenic composition for administration to an individual. The preparation of a vaccine is generally well understood in the art, as exemplified by U.S. Pat. Nos. 4,608,251, 4,601,903, 4,599,231, 4,599,230, and 4,596,792, all incorporated herein by reference. In particular embodiments, the compositions of the present invention are prepared to be pharmacologically acceptable vaccines.

In some embodiments, pharmaceutical vaccine or immunogenic compositions of the present invention comprise an effective amount of a polypeptide dimer (or nucleic acid or viral vector encoding the same) dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). In some embodiments, the antigen may comprise different types of carriers depending on whether it is to be administered in solid, liquid or aerosol form, and whether it need to be sterile for such routes of administration as injection. Except insofar as any conventional carrier is incompatible with the active ingredient, its use in the therapeutic or pharmaceutical compositions is contemplated.

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In some embodiments, sterile injectable solutions can be prepared by incorporating the antigens in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose.

The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein.

In particular embodiments, prolonged absorption of an injectable composition can be brought about by the use in the compositions of agents delaying absorption, such as, for example, aluminum monostearate, gelatin or combinations thereof.

For a broad overview of controlled delivery systems, see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nanospheres, and nanoparticles. Microcapsules can contain the therapeutically active agents as a central core. In microspheres the therapeutic can be dispersed throughout the particle. Particles, microspheres, and microcapsules smaller than about 1 μm are generally referred to as nanoparticles, nanospheres, and nanocapsules, respectively. Microparticles are typically around 100 m in diameter. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

In some embodiments, polymers can be used for controlled release of compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). In yet another aspect, liposomes can be used for controlled release as well as drug targeting of the lipid-capsulated drug (Betageri et al., Liposome Drug Delivery Systems, Technomic Publishing Co., Inc., Lancaster, Pa. (1993)).

### Methods

In another embodiment, the invention provides a method of inducing an immune response in a subject, comprising administering to the subject a pharmaceutical composition comprising an effective amount of a polypeptide or polypeptide dimer as described herein.

In some embodiments, the pharmaceutical composition comprises an effective amount of a polypeptide dimer comprising a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide.

In another embodiment, the invention provides a method of inducing an immune response in a subject, comprising administering to the subject a pharmaceutical composition comprising an effective amount of a nucleic acid or vector encoding a modified flavivirus envelope E polypeptide as described herein.

In some embodiments, the nucleic acid or vector encodes a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide.

As used herein, an “immune response” is the physiological response of the subject's immune system to an immunizing composition. An immune response may include an innate immune response, an adaptive immune response, or both. In one embodiment of the present invention, the immune response is a protective immune response. A protective immune response confers immunological cellular memory upon the subject, with the effect that a secondary exposure to the same or a similar antigen is characterized by one or more of the following characteristics: shorter lag phase than the lag phase resulting from exposure to the selected antigen in the absence of prior exposure to the immunizing composition; production of antibody which continues for a longer period than production of antibody resulting from exposure to the selected antigen in the absence of prior exposure to the immunizing composition; a change in the type and quality of antibody produced in comparison to the type and quality of antibody produced upon exposure to the selected antigen in the absence of prior exposure to the immunizing composition; a shift in class response, with IgG antibodies appearing in higher concentrations and with greater persistence than IgM, than occurs in response to exposure to the selected antigen in the absence of prior exposure to the immunizing composition; an increased average affinity (binding constant) of the antibodies for the antigen in comparison with the average affinity of antibodies for the antigen resulting from exposure to the selected antigen in the absence of prior exposure to the immunizing composition; and/or other characteristics known in the art to characterize a secondary immune response.

The term “subject” as used herein is not limiting and is used interchangeably with patient. In some embodiments, the term subject refers to animals, such as mammals and the like. For example, mammals contemplated include humans. primates, dogs, cats, sheep, cattle, goats, pigs, horses, chickens, mice, rats, rabbits, guinea pigs, and the like.

In some embodiments, the subject is a human.

In some embodiments, the pharmaceutical composition is administered to the subject more than one time. In some embodiments, the subject is administered a first priming dose of the pharmaceutical composition, and is subsequently boosted with a second dose of the pharmaceutical composition after the first priming dose. In some embodiments, the second dose is administered from 2-12 weeks after the first dose. In some embodiments, the second dose is administered from 2-8 weeks after the first dose. In some embodiments, the second dose is administered from 2-4 weeks after the first dose.

In some embodiments, the composition is administered via a parenteral route. In some embodiments, the parenteral route is selected from the group consisting of subcutaneous (s.c), intradermal (i.d.), intramuscular (i.m.), intraperitoneal (i.p.), and intravenous (i.v.) injection.

Any of the pharmaceutical compositions described herein may be administered to a subject with, prior to, or after administration of one or more adjuvants.

The pharmaceutical compositions described herein may be administered to a subject concomitantly with one or more vaccines to another infectious agent, such as another infectious agent is that present or thought to be present in the same geographic area as Zika virus or any of the other flaviviruses described herein. In some embodiments, the other infectious agent is one that the subject is also at risk of being in contact with. In some embodiments, the other infectious agent is transmitted by the same arthropod vector as Zika virus or any of the other flaviviruses described herein. In some embodiments, the other infectious agent is Japanese Encephalitis virus, Yellow Fever virus, Dengue virus and/or Chikungunya virus.

In some embodiments, the method further comprises assaying a sample from the subject after administering the immunizing composition. In some embodiments, the assaying comprises detecting the presence of a flavivirus pathogen using nucleic acid amplification tests which will determine if the subject is actively infected by a pathogen. In some embodiments, the assaying comprises detecting the presence of an immune response in the subject against the immunizing composition. In some embodiments, the detection is performed by serology tests. In some embodiments, the serology tests are performed by isolating a blood sample and running ELISA tests. In some embodiments, microfluidic systems are employed to run serology tests.

A vaccination or immunizing composition delivery schedule and dosages may be varied on a patient by patient basis, taking into account, for example, factors such as the weight and age of the patient, the type of disease being treated, the severity of the disease condition, previous or concurrent therapeutic interventions, the manner of administration and the like, which can be readily determined by one of ordinary skill in the art.

A vaccine or immunizing composition may be administered in a manner compatible with the dosage formulation, and in such amount as will be therapeutically effective and immunogenic. For example, the intramuscular route may be preferred in the case of toxins with short half lives in vivo. The quantity to be administered depends on the subject to be treated, including, e.g., the capacity of the individual's immune system to synthesize antibodies, and the degree of protection desired. The dosage of the vaccine will depend on the route of administration and will vary according to the size of the host. Precise amounts of an active ingredient required to be administered depend on the judgment of the practitioner.

In certain embodiments, pharmaceutical compositions may comprise, for example, at least about 0.1% by weight of an active compound. In other embodiments, the active compound may comprise between about 2% to about 75% of the weight of the unit, or between about 25% to about 60%, for example, and any range derivable therein. However, a suitable dosage range may be, for example, of the order of several hundred micrograms active ingredient per vaccination. Proper dosages of the polypeptides or heat killed or attenuated pathogens can be determined without undue experimentation using standard dose-response protocols.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per vaccination, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. A suitable regime for initial administration and booster administrations (e.g., innoculations) are also variable, but are typified by an initial administration followed by subsequent inoculation(s) or other administration(s).

In many instances, it will be desirable to have multiple administrations of the vaccine or immunizing composition, usually not exceeding six vaccinations, for example, more usually not exceeding four vaccinations and in some cases one or more, usually at least about three vaccinations. The vaccinations may be at from two to twelve-week intervals, more usually from three to five week intervals, although longer intervals are encompassed herein. Periodic boosters at intervals of 1-5 years, usually three years, may be desirable to maintain protective levels of the antibodies.

The course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays.

### Kits

Any of the compositions described herein may be comprised in a kit. The immunizing components of the kit may be packaged either in aqueous media or in lyophilized form. The kits of the present invention also will typically include a means for containing the composition and any other reagent containers in close confinement for commercial sale. Such containers may include injection or blow-molded plastic containers into which the desired vials are retained.

The component(s) of the kit may be provided as dried powder(s). When reagents and/or components are provided as a dry powder, the powder can be reconstituted by the addition of a suitable solvent. It is envisioned that the solvent may also be provided in another container means. The kits may comprise a container means for containing a sterile, pharmaceutically acceptable buffer and/or other diluent.

In some embodiments the kits can be for use in prophylactically administering to a subject, for example to prevent or reduce the severity of flavivirus infection. Such kits can include one or more containers comprising a composition containing nucleic acids, vectors, or polypeptide dimers as described herein. In some embodiments, the kit may further comprise a second composition, such as a second vaccine. In some embodiments, the second vaccine is a vaccine for another flavivirus. In some embodiments, the second vaccine is a Dengue virus vaccine and/or a Chikungunya virus vaccine.

In some embodiments, the kit can comprise instructions for use in accordance with any of the methods described herein. The included instructions can comprise a description of administration of the composition containing the nucleic acids, vectors or polypeptide dimers to prevent, delay the onset, or reduce the severity of flavivirus infection. The kit may further comprise a description of selecting a subject suitable for administration based on identifying whether that subject is at risk for exposure to flavivirus or contracting a flavivirus infection. In still other embodiments, the instructions comprise a description of administering a composition to a subject at risk of exposure to flavivirus or contracting flavivirus infection.

The instructions relating to the use of the composition generally include information as to the dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine readable instructions are also acceptable.

The kits of the present disclosure are in suitable packaging. Suitable packaging includes, but is not limited to, vials, bottles, jars, flexible packaging, and the like. Also contemplated are packages for use in combination with a specific device, such as a syringe or an infusion device. The container may have a sterile access port, for example the container may be a vial having a stopper pierceable by a hypodermic injection needle. At least one active agent in the composition is an inactivated flavivirus, as described herein.

While the present teachings are described in conjunction with various embodiments, it is not intended that the present teachings be limited to such embodiments.

On the contrary, the present teachings encompass various alternatives, modifications, and equivalents, as will be appreciated by those of skill in the art.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. Structure-Based Vaccine Design of ZIKV E Proteins to Selectively Enhance Quaternary nAb Epitope Presentation and Dampen ADE-Prone Epitopes

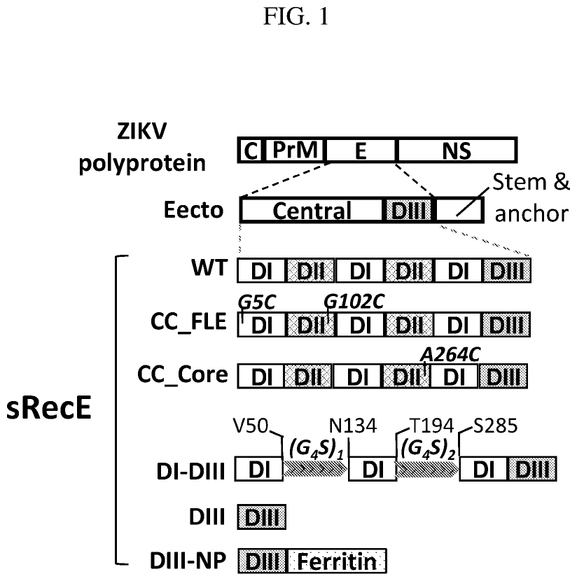

The ZIKV E protein presents as a dimeric form consisting of two protomers on the mature vial surface and recognizes quaternary epitope EDE-directed nAbs with high affinity (Barba-Spaeth G et al., Nature, (2016), 536:48-53; Fernandez et al., Nat Immunol, (2017), 18:1261-1269). Recombinant soluble form of ZIKV E protein, namely sRecE, expresses predominantly as a monomer, binds EDE nAbs poorly, and shows high affinity for FLE-directed ADE-prone non-neutralizing antibodies. Conceptually, the monomeric form of sRecE is not an ideal immunogen for stimulating protective immune response due to (i) the poor presentation of quaternary EDE nAb epitopes which are more readily formed in dimeric context, and (ii) the extensive exposure of weak/non-neutralizing FLE epitope, which could robustly elicits ADE-prone antibodies. Therefore, we first sought to engineer stable ZIKV dimers to mimic native E protein conformation as vaccine candidate, by introducing one or two inter-protomer disulfide bonds in sRecE, based on structural analysis (PDB: 5JHM). The corresponding recombinant E protein constructs were designated as sRecE_CC_FLE carrying mutations G5C/G102C and sRecE_CC_Core carrying mutation A264C, respectively (FIG. 1). Of note, the mutation of CC_FLE located within FLE (G102C) could also dampen the antigenicity of FLE by tethering the FLE on one protomer to the N-terminus of the other protomer and reduce ADE effect.

In addition, we took an alternative strategy to abolish potential ADE antibody elicitation by (i) deleting DII or (ii) deleting DI & DII that contain more conserved amino acid residues, while retaining domain III as the major nAb epitope, which resulted in construct DI-DIII, and DIII, respectively (FIG. 1). For improved immunogenicity (Bachmann M F et al., Nat Rev Immunol, (2010), 10:787-96; Rehm B H A. et al., Curr Opin Biotechnol, (2017), 48:42-53; Frietze K M et al., Curr Opin Virol, (2016), 18:44-9), we fused the C-terminus of DIII to the N-terminus encoding gene fragment of a self-assembling virus-like nanoparticle (NP), ferritin (13), resulting in construct DIII-NP as vaccine candidate (FIG. 1).

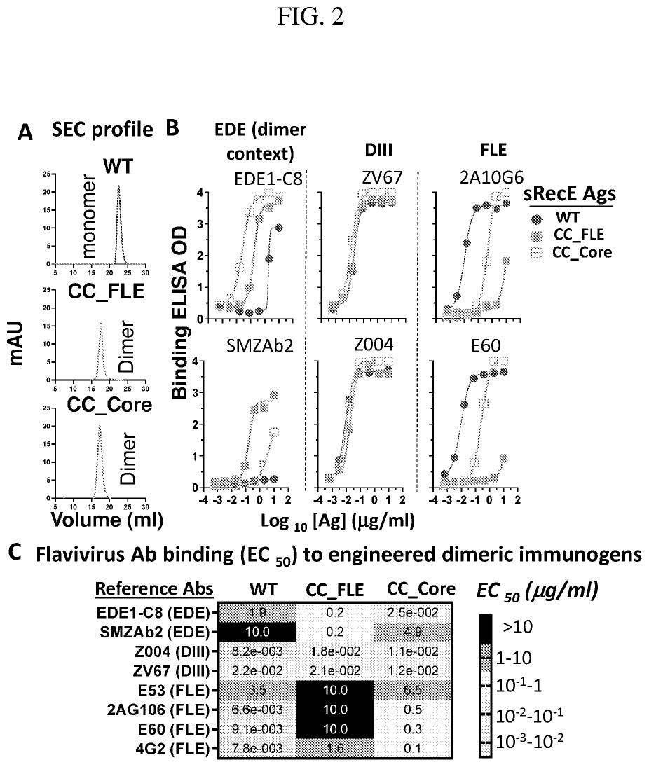

We synthesized the modified sRecE immunogen encoding genes (FIG. 1) and cloned them into the BglII/AgeI site of Drosophila pMT/BiP/V5-His B vector (ThermoFisher), followed by transfecting and selecting transfected Drosophila S2 cells (ThermoFisher). We expressed the immunogen proteins, purified by affinity column packed with cOmplete His-Tag Purification Resin (Roche), followed by Size Exclusion Chromatography (SEC) purification using Superose 6 Increase 10/300 GL column (Cytiva). The two purification steps resulted in dimeric CC_FLE and CC_Core sRecE proteins with high homogeneity (FIG. 2A), as expected. To characterize the antigenicity of our novel immunogens, we tested their binding affinity for a panel of reference mAbs, in comparison with WT sRecE. Consistent with the design rationale, WT sRecE in majority of monomeric form binds EDE quaternary epitope mAbs poorly; CC_FLE binds both EDE mAbs, EDE1-C8 and SMZAb2 well, while CC-Core only binds the former but not the latter mAb (FIG. 2B). The DIII LR epitopes are well retained by both CC_FLE and CC-Core, as they bind DIII LR epitope mAbs, ZV67 and Z004 well, similar to WT (FIG. 2B). Lastly, as expected, CC_FLE has nearly abolished binding to FLE mAbs 2A10G6 and E60 (FIG. 2B), while CC_Core shows significantly dampened binding compared to WT. We assessed the reactivity of more flavivirus FLE cross-reactive but weak or non-neutralizing mAbs (FIG. 2C), and consistently noted that our dimeric mutant proteins containing inter-protomer CC bonds (i) remarkably show enhanced EDE nAb epitope presentation compared to WT sRecE; (ii) well retain nAb epitopes on domain III; and (iii) all substantially dampen the display of FLE epitopes (FIG. 2C).

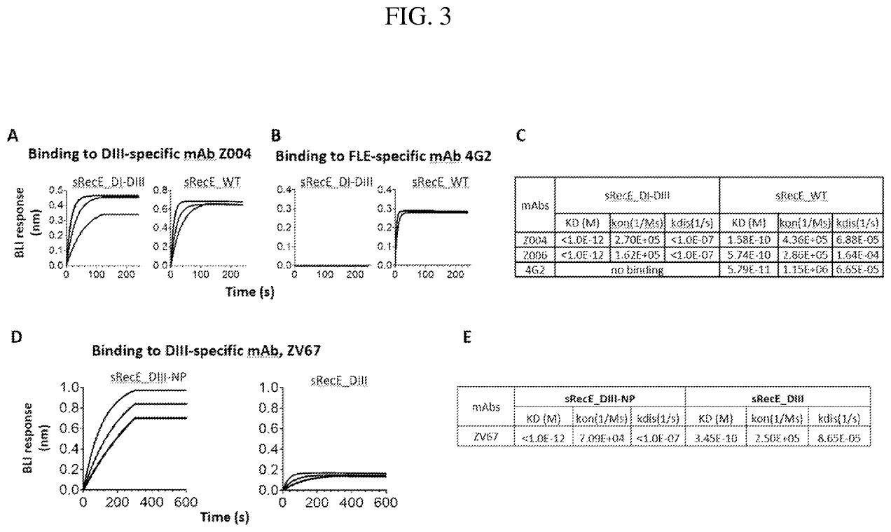

As expected, the construct DI-DIII bound a panel of DIII-specific nAbs including Z004 & Z006 well (FIG. 3A-C) without binding activity to FLE-specific mAb, 4G2 (FIG. 3B & C). The constructs, DIII and DIII-NP bind DIII-specific nAb ZV67 well (FIG. 3D-E) in a manner similar to WT sRecE protein, indicating well retained antigenicity.

### CC_FLE Elicits Potent nAb Response in Immune Mice

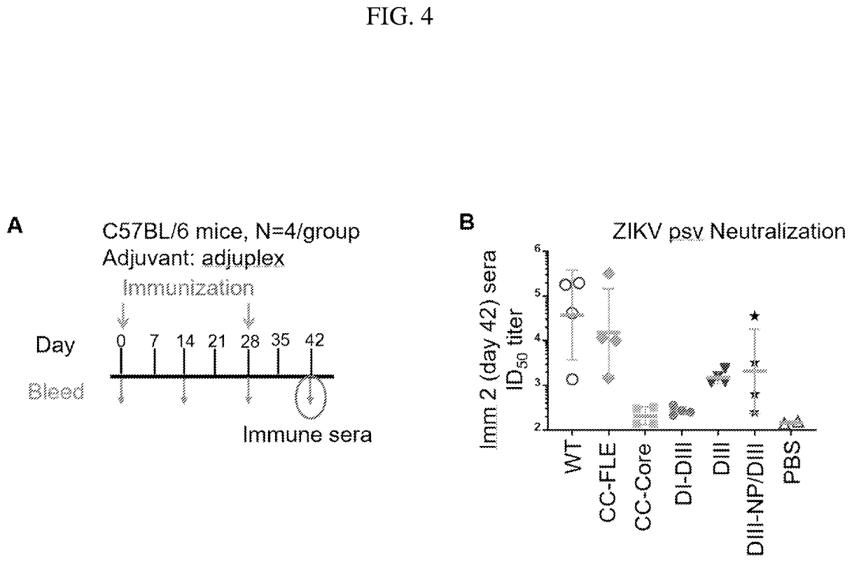

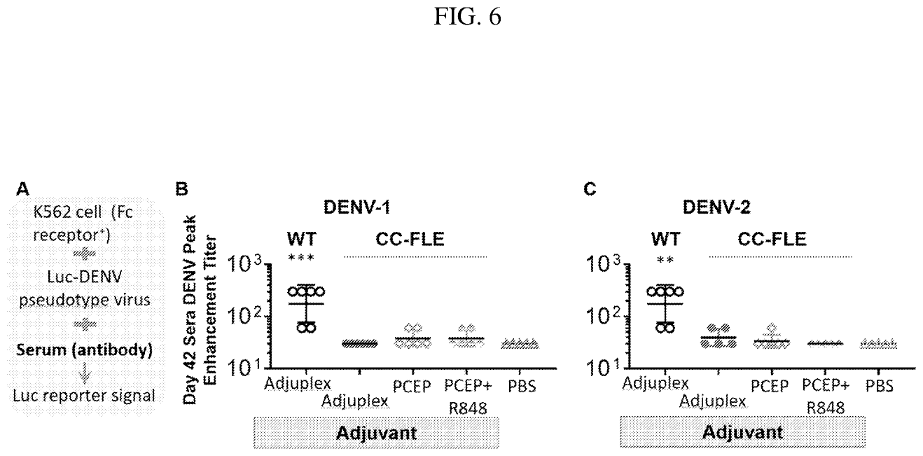

We set to test the immunogenicity of the engineered vaccine candidates described above in mice (FIG. 4A). After two immunizations, sera from mice immunized with CC_FLE showed geometric mean of ID₅₀ titers (˜10⁴) similar to WT sRecE (FIG. 4B), while mice immunized with DIII or primed with DIII-NP/boosted with DIII showed 10-fold lower ID₅₀ titers (FIG. 6B), suggesting that the deletion of DI and DII of sRecE leads to attenuated immunogenicity. Surprisingly, the CC_Core dimer has elicited very low nAb titers nearly equivalent to DI-DIII and PBS inoculated mice group (FIG. 6B), indicating that dimeric context alone is not sufficient for robust immunogenicity. Nevertheless, the observed robust nAb response from the immune mice inoculated with CC_FLE dimer indicated premise for further development.

### Novel Polyphosphazene-Based Adjuvant Formulation Potentiates Vaccine Efficacy in Mice

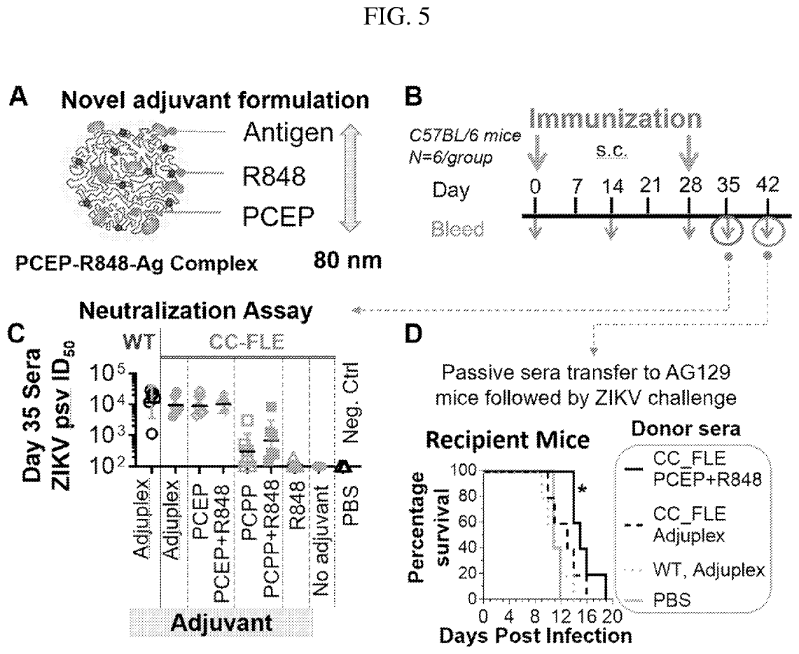

Polyphosphazene (PPZ) adjuvants (FIG. 5A) are water-soluble synthetic macromolecules with phosphorus-nitrogen backbone and organic side groups, which can degrade in the body resulting in benign products (Andrianov et al., J Controlled Release, (2021), 329:299-315; Powell et al., Clin Exp Vaccine Res, (2015), 4:23-45). PPZ adjuvants are able to stimulate accelerated onset of the immune response, prolonged immunity, and to modulate quality of the immune response (Andrianov et al., J Controlled Release, (2021), 329:299-315). The 1^{st} generation PPZ product candidate—PCPP has shown excellent safety profile in five clinical trials (Andrianov et al., J Controlled Release, (2021), 329:299-315). Other derivatives, such as PCEP, demonstrated ability to both enhance and modulate quality of the immune response (Mutwiri et al., Vaccine, (2007), 25:1204-1213; Andrianov A K et al., Biomacromolecules, (2006), 7:394-399). R-848 is a TLR7/8 agonist being advanced to clinical trials that activates immune responses in TLR7/8 dependent mechanism and induces superior cytokine secretion, macrophage activation, and enhancement of cellular immunity (Tomai M A et al., Expert Rev Vaccines, (2011). 10:405-407; Vasilakos et al., Expert Rev Vaccines, (2013), 12:809-819; Tomai M A et al., Expert Rev of Vaccines, (2007), 6:835-847). We have recently further advanced PPZ platform by co-assembling antigen-PPZ complexes with R848, which resulted in supramolecular constructs that mimic virus-like characteristics desirable for stimulating potent immune response: (i) 60-80 nm diameter, (ii) repetitive/multiple antigen display, and (iii) decoration with danger signals recognized by pattern recognition receptors (Andrianov A K et al., ACS Appl Bio Mater, (2020), 3:3187-3195)(FIG. 5A).

Thus, we attempted to test if PPZ in conjunction with R848 could better potentiate the immunogenicity of sE protein than adjuplex, the adjuvant we used previously. We focused on investigating the immunogenicity of our lead immunogen CC_FLE in various PPZ formulations including (i) PCEP, (ii) PCEP+R848, (iii) PCPP, (iv) PCPP+R848, (v) R848, and (vi) adjuplex as control. We also added WT sRecE formulated with adjuplex as reference group, in the immunization study depicted in FIG. 5B. Sera on Day 35 (1 week after the 2^{nd} immunization) from mice immunized with WT sRecE formulated with adjuplex showed neutralization ID₅₀ titers against ZIKV pseudotype virus around 10⁴, similar to mice immunized with CC_FLE formulated with adjuplex, PCEP, or PCEP+R848 (FIG. 5C), while sera from immune mice inoculated with CC_FLE formulated with PCPP or PCPP+R848 displayed 20-30 fold lower ID₅₀ titers (FIG. 5C). No neutralization activity was detectable in sera from mice immunized with CC_FLE formulated with R848, or CC_FLE alone (no adjuvant).

We then sought to determine the protective efficacy of selected sera from the immune mice group with the highest ID₅₀ neutralization titers (FIG. 5C). In a passive sera transfer study (FIG. 5D), AG129 mice (IFNα/β/γR−/−), immunocompromised and highly susceptible to ZIKV infection, were infused with 200 μl of donor C57BL/6 mouse immune sera of day 42 (FIG. 5B) via intraperitoneal (i.p.) route, challenged with ZIKV FSS13025 via s.c. route 1 hour post serum infusion, and were monitored for 20 days post infection (DPI). AG129 mice receiving sera from immune mice inoculated with CC_FLE formulated with PCEP+R848 adjuvant displayed significantly longer survival time (median, 15 days) upon challenge than from animals inoculated with PBS (median, 11 days) (* p<0.05, Mantel-Cox log-rank test) or WT sRecE formulated with adjuplex (median, 11 days) (FIG. 5D). AG129 mice receiving sera from donor mice immunized with CC_FLE formulated with adjuplex also showed median survival time of 13 days (FIG. 5D), longer than PBS- or WT sRecE+adjuplex inoculated group, although this is not statistically significant. In summary, formulation of our novel immunogen, CC_FLE with PCEP+R848 is beneficial for potentiating immunogen protection efficacy, indicated by (i) potent nAb titer of immune sera (FIG. 5C) and (ii) in vivo protection for ZIKV challenged AG129 mice conferred by passive immune sera transfer (FIG. 5D).

### CC_FLE Mutation Largely Abolishes ADE Potential for Enhancing DENV Infection

To further evaluate the potential of CC_FLE inducing cross-reactive ADE-prone Abs, we incubated DENV serotype 1 (DENV-1) or serotype 2 (DENV-2) pseudotype virus with sera from CC_FLE or WT sRecE immune mice (FIG. 5B), followed by co-incubation with K562 cells (_{bu}FcγRIIA+) to mimic Fe receptor-mediated ADE for DENV infection (FIG. 6A). While most sera from mice immunized with WT sRecE formulated with adjuplex showed enhancement titer for DENV-1 or DENV-2 around 200 (FIG. 6A-B), most sera from mice immunized with CC-FLE formulated with adjuplex or other PCEP-based adjuvant showed no enhancement for DENV-1 or DENV-2 (FIG. 6A-B).

### CC_FLE Immunization Confers Nearly Complete Protection in Immunocompetent Mice

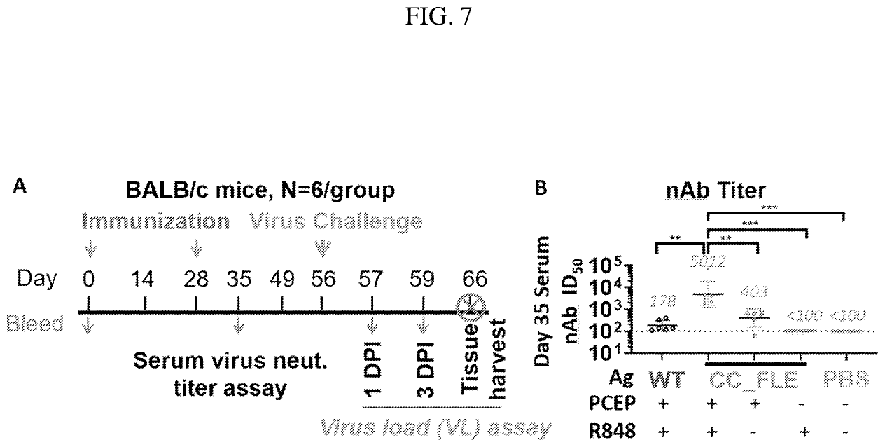

We set to evaluate the protection efficacy of our lead immunogen CC_FLE by immunizing immunocompetent BALB/c mice with CC_FLE formulated in the optimal adjuvant combination PCEP+R848, with WT sRecE as control, followed by ZIKV challenge (FIG. 7A). To evaluate individual adjuvant effect, we also immunized mice with CC_FLE formulated in either PCEP or R848 to evaluate individual adjuvant effect. All mice were immunized and challenged as depicted in FIG. 7A. We chose BALB/c mice as the model animal for vaccine efficacy here based on our in-house observation that BALB/c mice challenged by ZIKV in general develop higher titers of viremia than C57BL/6 mice (the mouse strain we used for immunogenicity studies earlier), suggesting BALB/c mouse as a more suitable model animal for evaluating vaccine efficacy than C57BL/6 mouse.

We first assessed the nAb titers of the day 35 immune mouse sera. Sera from mice inoculated with WT sRecE formulated in PCEP+R848 displayed moderate ID₅₀ titers (geometric mean=178) (FIG. 7B), whereas mouse sera from CC_FLE formulated in PCEP+R848 group had ID₅₀ titers (geometric mean=5,012) 2-logs higher than the WT-sRecE group (FIG. 7B). CC_FLE formulated in single adjuvant PCEP and R848 elicited moderate and negligible nAb responses, respectively (FIG. 7B). In earlier studies (FIGS. 4 & 5), we observed that WT sRecE and CC_FLE elicited similar nAb titers in C57BL/6 mice, which is somehow different from the result here in BALB/c mice. The use of different mouse strains may cause the discrepancy. However, our nAb titers in BALB/c mice inoculated with monomer WT-sRecE are consistent with historic data from the literature (Slon-Campos et al., Nat Immunol, (2019), 20:1291-1298), which suggests that CC_FLE formulated in PCEP+R848 is able to elicit nAb responses superior to WT-sRecE in the same adjuvant in BALB/c mice, and similar to WT-sRecE in C57BL/6 mice. Therefore, CC_FLE formulated in PCEP+R848 serves as an immunization regimen capable of eliciting nAb responses more consistent than the monomer WT sRecE in various mouse strains.

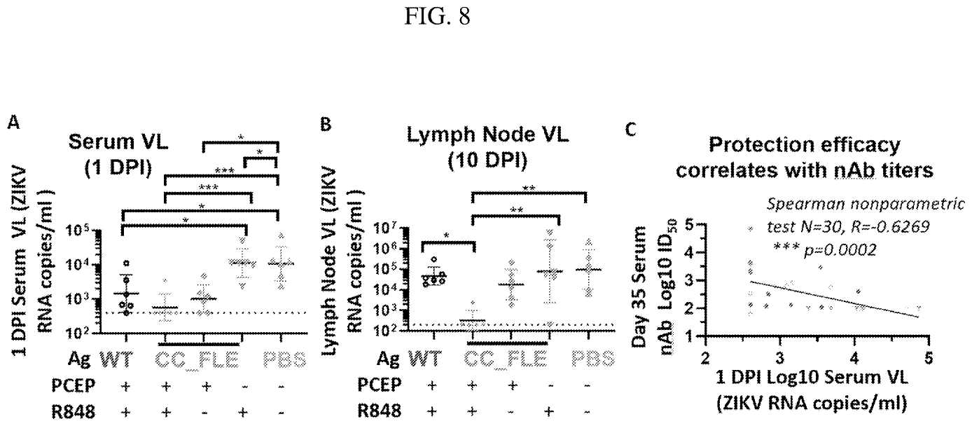

To test if the observed potent nAb titers elicited by CC_FLE formulated in PCEP+R848 could translate into in vivo protection, we challenged the immune mice on day 56 (1 month after the 2^{nd} immunization) (FIG. 8A) with ZIKV, and monitored serum virus load (VL) afterwards. As the negative control for immunization, 1 DPI sera from PBS-inoculated mice showed average 10⁴ copies of ZIKV RNA/ml. In contrast, 83% (5/6) of mice immunized with CC_FLE formulated in PCEP+R848 showed no detectable serum VL (FIG. 8A). WT sRecE formulated in PCEP+R848 and CC_FLE formulated in PCEP alone provided partial protection, with nearly all mouse sera displayed detectable ZIKV RNA (geomean ˜10³ ZIKV RNA copies/ml), 10-fold lower than PBS-inoculated mice (FIG. 8A). Consistent with 1 DPI serum VL result, ZIKV RNA could be detected in tissues including lymph node (FIG. 8B) and spleen (not shown) in all animal groups (average 10⁴ ZIKV RNA copies/ml) except that only one mouse in the mouse group immunized with CC_FLE formulated in PCEP+R848 showed detectable ZIKV RNA (FIG. 8B). Furthermore, there is an inverse correlation of pre-infection serum nAb titers and 1 DPI serum virus load (FIG. 8C), suggesting nAb response is the key factor contributing to protection efficacy.

Taken together, our lead immunogen, CC_FLE formulated in PCEP and R848 adjuvant complex is able to (i) elicit potent nAb responses thus confer nearly complete protection in ZIKV challenged mice (FIGS. 7 & 8), and (ii) minimize stimulating ADE-prone Ab responses (FIG. 6), superior to the nascent WT sRecE. It is conceivable that CC_FLE may elicit nAb response more focused on the major nAb epitopes such as the quaternary EDE and DIII LR while avoiding stimulating non-neutralizing ADE-prone antibody responses, consistent with the initial rationale of structure-based design. This unique novel immunogen and adjuvant combination has potential to be advanced to licensed vaccine in future preclinical and clinical studies. In addition, the mutations.

G5C/G102C, carried by CC_FLE could be applied to various ZIKV vaccine platforms, including the mRNA form to serve as vaccine candidates with improved efficacy and safety.

### Example 2. CC_FLE Linkage can be Applied to the sEs of Other Flaviviruses to Prevent ADE

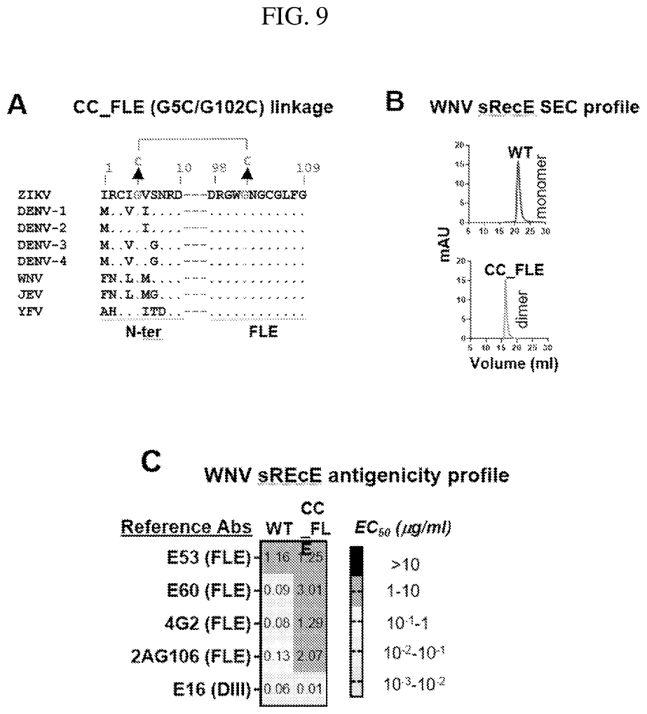

Sequence homology analysis of the N-terminus and FLE regions of the sEs of a number of mosquito-borne flaviviruses revealed that both residues G5 and G102 that were mutated to C (cysteine) in the ZIKV CC_FLE design to form N-term/FLE disulfide linkage present in the corresponding sEs (FIG. 9A). The potential applicability of this disulfide bond linkage in the context of sEs of other flaviviruses such as WNV was subsequently corroborated by structural analysis of the WNV envelope proteins presented in WNV particle (PDB: 3IYW) that predicted high likelihood of this disulfide bond formation. We then expressed and characterized the CC_FLE version of WNV sE, which showed dimeric configuration after purification (FIG. 9B), in contrast to the predominantly monomeric WNV WT sE (FIG. 9B). As expected, WNV CC_FLE sE displayed 10-30-fold decreased binding to FLE-specific mAbs, in comparison with the WT sE (FIG. 9C). Therefore, the CC_FLE design strategy could potentially be applied to prevent ADE of sE-based vaccines of other flaviviruses.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1. A polypeptide comprising a modified flavivirus envelope E polypeptide, wherein the modified flavivirus envelope E polypeptide comprises i) a cysteine residue in a fusion loop epitope of ectodomain DII; and ii) a cysteine residue in ectodomain DI located in an N-terminal portion of the polypeptide.

2. The polypeptide of claim 1, wherein the polypeptide is present in a dimer, wherein the dimer consists of two modified flavivirus envelope E polypeptides that have the same amino acid sequence.

3. The polypeptide of claim 2, wherein the cysteine residue in the fusion loop epitope of ectodomain DII on one polypeptide chain forms an interchain disulfide bond with the cysteine residue in the ectodomain DI on the other polypeptide chain.

4. The polypeptide of claim 1, wherein the modified flavivirus envelope E polypeptide comprises ectodomains DI, DII, and DIII, and lacks stem and anchor domains.

5. The polypeptide of claim 1, wherein the cysteine residues are present at amino acid position numbers 5 and 102 with reference to any of SEQ ID NOS:1, 7, 20,24, 28, 32, 36, 40, 44, 48, 52, 56, 60, 64, 68, or 72.

6. The polypeptide of claim 1, wherein the modified flavivirus envelope E polypeptide comprises an amino acid sequence at least 90% identical to any of SEQ ID NOS:2, 8, 17, 21, 25, 29, 33, 37, 41, 45, 49, 53, 57, 61, 65, 69, or 73, or an antigenic fragment thereof.

7. The polypeptide of claim 1, wherein the modified flavivirus envelope E polypeptide comprises an amino acid sequence of any of SEQ ID NOS:2, 8, 17, 21, 25, 29, 33, 37, 41, 45, 49, 53, 57, 61, 65, 69, or 73, or an antigenic fragment thereof.

8. The polypeptide of claim 1, wherein the flavivirus is a Zika virus.

9. The polypeptide of claim 8, wherein the modified Zika virus envelope E polypeptide comprises an amino acid sequence of SEQ ID NO:2.

10. The polypeptide of claim 1, wherein the flavivirus is a West Nile virus.

11. The polypeptide of claim 10, wherein the modified West Nile virus envelope E polypeptide comprises an amino acid sequence of SEQ ID NO:8.

12. A pharmaceutical composition comprising an effective amount of a polypeptide of claim 1.

13-19. (canceled)

20. A nucleic acid encoding a modified flavivirus envelope E polypeptide of claim 1.

21. (canceled)

22. A vector encoding the modified flavivirus envelope E polypeptide of claim 1.

23. The vector of claim 22, wherein the vector is a viral vector.

24. (canceled)

25. A host cell comprising the nucleic acid of claim 20.

26. A pharmaceutical composition comprising an effective amount of the nucleic acid of claim 20.

27. The pharmaceutical composition of claim 26, wherein the nucleic acid is formulated as a nanoparticle.

28. The pharmaceutical composition of claim 27, wherein the nanoparticle is a lipid nanoparticle.

29. A method of inducing an immune response in a subject, comprising administering to the subject the pharmaceutical composition of claim 12.

30-34. (canceled)
